(19) United States
(12) Patent Application Publication
(10) Pub. No.: US 2025/0011843 A1
(43) Pub. Date: Jan. 9, 2025

(54) MOLECULAR DETECTION USING BARCODE PROBES OR BARCODE FUSION PROBES AND HYBRIDIZATION CHAIN REACTION

(71) Applicant: California Institute of Technology, Pasadena, CA (US)

(72) Inventors: Kai Zinn, Pasadena, CA (US); Shuwa Xu, Glendale, CA (US); Michael Anaya, Pasadena, CA (US); Sheldon A. Zhu, Pasadena, CA (US); Niles A. Pierce, Pasadena, CA (US)

(21) Appl. No.: 18/764,002

(22) Filed: Jul. 3, 2024

Related U.S. Application Data
(60) Provisional application No. 63/525,590, filed on Jul. 7, 2023.

(51) Int. Cl. C12Q 1/682; C12Q 1/6818

(52) U.S. Cl. CPC C12Q 1/682; C12Q 1/6818

(57) ABSTRACT

The present application relates to hybridization chain reaction (HCR). The sensitivity and multiplexing of hybridization chain reaction (HCR) signal amplification are combined with target detection using barcode probes or barcode fusion probes, wherein a barcode probe or barcode fusion probe comprises a primary probe (comprising a target-binding domain) bound to a secondary probe (comprising a primary-probe-binding-domain and a barcode oligonucleotide comprising an HCR initiator or an HCR fractional initiator).

### REFERENCE TO RELATED APPLICATIONS

This application claims priority to U.S. Provisional Patent Application No. 63/525,590, filed Jul. 7, 2023, the disclosures of which are hereby incorporated by reference in their entirety.

### STATEMENT REGARDING FEDERALLY SPONSORED R&D

This invention was made with government support under Grant Nos. R01EB006192 and R01EY028116 awarded by the National Institutes of Health. The government has certain rights in the invention.

### INCORPORATION BY REFERENCE OF MATERIAL IN SEQUENCE LISTING FILE

This application incorporates the material in the XML sequence listing provided herewith, entitled CALTE.¹⁷¹Aseqlist.xml, was created on Jul. 3, 2024, and is 3,045 bytes in size.

### BACKGROUND

### Field

The present application relates to hybridization chain reaction (HCR). The sensitivity and multiplexing of hybridization chain reaction (HCR) signal amplification are combined with target detection using barcode probes or barcode fusion probes, wherein a barcode probe or barcode fusion probe comprises a primary probe (comprising a target-binding domain) bound to a secondary probe (comprising a primary-probe-binding-domain and a barcode oligonucleotide comprising an HCR initiator or an HCR fractional initiator). Because the barcode probes or barcode fusion probes are pre-assembled prior to performing a multiplex assay, there is no need for the primary probes (e.g., primary antibodies) for different targets to be raised in different host organisms or to be of different isotypes, and the secondary probe can comprise the same primary-probe-binding domain for all targets together with a unique barcode oligonucleotide for each target, mediating generation of a distinguishable amplified HCR signal for each target. Furthermore, because each secondary probe comprises a barcode oligonucleotide, there is no need to conjugate a barcode oligonucleotide to a primary probe for each new target, eliminating challenges associated with oligonucleotide conjugation interfering with primary probe binding of the target.

### SUMMARY

In accordance with some implementations, a barcode probe is provided comprising: a) a primary probe comprising a primary recognition domain comprising a primary antibody, antibody fragment, nanobody, or polypeptide, wherein the primary antibody, antibody fragment, nanobody, or polypeptide comprises a target-binding domain, b) a secondary probe comprising: i) a secondary recognition domain comprising a secondary antibody, antibody fragment, nanobody, or polypeptide, wherein the secondary antibody, antibody fragment, nanobody, or polypeptide comprises a primary-probe-binding domain and a conjugation site, and ii) a barcode oligonucleotide. In some embodiments, the conjugation site is covalently coupled to the barcode oligonucleotide. In some embodiments, the secondary probe is bound to the primary probe. In accordance with some implementations, a barcode probe comprises a barcode oligonucleotide comprising an HCR initiator or an HCR fractional initiator.

In accordance with some implementations, a method of constructing a barcode probe is provided comprising performing any of the following steps in any order: a) combining a barcode oligonucleotide with a secondary recognition domain comprising a primary-probe-binding domain and a conjugation site; and covalently coupling the conjugation site to the barcode oligonucleotide thereby forming a secondary probe, b) combining the secondary probe with a primary probe comprising a primary recognition domain comprising a target-binding domain; whereupon the primary-probe-binding domain binds to the primary probe, thereby forming the barcode probe. In accordance with some implementations, the method comprises crosslinking the primary probe to the secondary probe. In accordance with some implementations, the method comprises purifying the barcode probe.

In accordance with some implementations, a barcode probe comprises a barcode oligonucleotide comprising an HCR initiator or an HCR fractional initiator. In accordance with some implementations, a barcode probe comprises a conjugation site comprising a lysine residue or another natural amino acid. In accordance with some implementations, a barcode probe comprises a conjugation site comprising a non-natural amino acid. In accordance with some implementations, a secondary probe comprises two or more conjugation sites each covalently coupled to a barcode oligonucleotide. In accordance with some implementations, the secondary recognition domain is expressed prior to covalent coupling of the conjugation site to the barcode oligonucleotide. In accordance with some implementations, the secondary recognition domain is purified prior to covalent coupling of the conjugation site to the barcode oligonucleotide. In accordance with some implementations, the secondary probe is purified prior to binding the secondary probe to the primary probe to form the barcode probe.

In accordance with some implementations, a panel of N barcode probes for detection of a panel of N targets is provided wherein N is a positive integer. In some embodiments, barcode probe j (for j=1, . . . , N) comprises: a) a primary probe j comprising a primary recognition domain j comprising a primary antibody, an antibody fragment, a nanobody, or a polypeptide, wherein the primary antibody, antibody fragment, nanobody or polypeptide comprises a target-binding domain j, b) a secondary probe j comprising: i) a secondary recognition domain j comprising a secondary antibody, an antibody fragment, a nanobody or a polypeptide comprising a primary-probe-binding domain j and a conjugation site j, and ii) a barcode oligonucleotide j. In accordance with some implementations, the conjugation site j is covalently coupled to the barcode oligonucleotide j. In accordance with some implementations, the secondary probe j is bound to the primary probe j.

In accordance with some implementations, a panel of N barcode probes for detection of a panel of N targets is provided, wherein N is a positive integer. In accordance with some implementations, barcode oligonucleotide j comprises an HCR initiator j or an HCR fractional initiator j for j=1, . . . , N. In accordance with some implementations, a panel of N barcode probes for detection of a panel of N targets is provided, wherein the same conjugation site is used for two or more barcode probes in the panel. In accordance with some implementations, a panel of N barcode probes for detection of a panel of N targets is provided, wherein the same secondary recognition domain is used for two or more probes in the panel. In accordance with some implementations, a panel of N barcode probes for detection of a panel of N targets is provided, wherein target-binding domain j is specific to target j and barcode oligonucleotide j is unique to target j for j=1, . . . , N.

In accordance with some implementations, a method is provided for amplified detection of a target in a sample, the method comprising: a) providing a sample containing a target; b) providing a probe set comprising either: i) an HCR initiator-labeled probe, or ii) a probe unit comprising two or more HCR fractional-initiator probes; c) providing a reporter-labeled first HCR amplifier; d) generating a signal, directly or indirectly, from one or more reporter-decorated HCR amplification polymers; c) detecting the signal. In accordance with some implementations, an HCR initiator-labeled probe comprises: a barcode probe comprising: i) a primary probe comprising target-binding domain and ii) a secondary probe comprising a primary-probe-binding domain and a conjugation site; wherein the secondary probe is bound to the primary probe and the conjugation site is covalently coupled to a barcode oligonucleotide comprising an HCR initiator. In accordance with some implementations, an HCR fractional-initiator probe comprises: a barcode probe comprising i) a primary probe comprising a target-binding domain and ii) a secondary probe comprising a primary-probe-binding domain and a conjugation site; wherein the secondary probe is bound to the primary probe and the conjugation site is covalently coupled to a barcode oligonucleotide comprising an HCR fractional initiator. In accordance with some implementations, an HCR amplifier comprises two or more HCR hairpins. In accordance with some implementations, an HCR hairpin comprises an input domain comprising a single-stranded toehold and a stem section. In accordance with some implementations, an HCR hairpin further comprises an output domain comprising a single-stranded loop and a complement to the stem section. In accordance with some implementations, at least one HCR hairpin further comprises one or more reporters. In accordance with some implementations, when and HCR initiator-labeled probe or probe unit is bound to the target, the HCR initiator or colocalized full initiator comprising two or more HCR fractional-initiators initiates HCR signal amplification whereupon the HCR hairpins self-assemble into a tethered reporter-decorated HCR amplification polymer thereby generating a signal. In accordance with some implementations, a wash step is performed between steps b) and c). In accordance with some implementations, a wash step is performed between steps c) and d). In accordance with some implementations, the signal is removed from the sample following step e). In accordance with some implementations, the reporters on the reporter-decorated amplification polymers directly or indirectly mediate CARD signal amplification. In accordance with some implementations, the target comprises a protein, a peptide, an amino acid, a non-natural amino acid analog, a nucleic acid, a non-natural nucleic acid analog, a tag, a hapten, a fluorophore, a reporter, a chemical, a biological molecule, a pathogen, a small molecule, a macromolecule, a complex of molecules, or a set of molecules in proximity. In accordance with some implementations, the probe unit comprising two or more HCR fractional-initiator probes further comprises one or more proximity probes. In accordance with some implementations, each HCR fractional-initiator probe within a probe unit further comprises a proximity domain. In accordance with some implementations, the one or more proximity probes bind to the proximity domains within a probe unit to colocalize a full HCR initiator capable of triggering HCR signal amplification. In accordance with some implementations, any of the steps of the method are repeated to detect another target in the sample.

In accordance with some implementations, a barcode fusion probe is provided comprising a primary probe comprising: a) a primary recognition domain comprising a primary antibody, an antibody fragment, a nanobody, or a polypeptide, wherein the primary antibody, antibody fragment, nanobody, or polypeptide comprises a target-binding domain, b) an enzymatic conjugation domain, and c) a barcode oligonucleotide; wherein the primary recognition domain is fused to the enzymatic conjugation domain, and wherein the enzymatic conjugation domain is covalently coupled to the barcode oligonucleotide.

In accordance with some implementations, a barcode fusion probe is provided, comprising: a) a primary probe comprising a primary recognition domain comprising a primary antibody, antibody fragment, nanobody, or polypeptide, wherein the primary antibody, antibody fragment, nanobody, or polypeptide comprises a target-binding domain, b) a secondary probe comprising: i) a secondary recognition domain comprising a secondary antibody, antibody fragment, nanobody, or polypeptide, wherein the secondary antibody, antibody fragment, nanobody, or polypeptide comprises a primary-probe-binding domain, ii) an enzymatic conjugation domain, and iii) a barcode oligonucleotide; wherein the secondary recognition domain is fused to the enzymatic conjugation domain, wherein the enzymatic conjugation domain is covalently coupled to the barcode oligonucleotide, and wherein the secondary probe is bound to the primary probe.

In accordance with some implementations, the barcode oligonucleotide comprises an HCR initiator or an HCR fractional initiator. In accordance with some implementations, the enzymatic conjugation domain comprises an HUH domain. In accordance with some implementations, the HUH domain is DCV.

In accordance with some implementations, a method of constructing a barcode fusion probe is provided, comprising: combining a barcode oligonucleotide with a fusion probe comprising: a) a primary recognition domain comprising a target-binding domain, and b) an enzymatic conjugation domain, whereupon the enzymatic conjugation domain covalently couples to the barcode oligonucleotide. In accordance with some implementations, a method of constructing a barcode fusion probe further comprises purifying the barcode fusion probe.

In accordance with some implementations, a method of constructing a barcode fusion probe is provided, comprising, performing the following steps in any order: a) combining a barcode oligonucleotide with a secondary recognition domain comprising a primary-probe-binding domain fusing to an enzymatic conjugation domain; whereupon the enzymatic conjugation domain covalently couples to the barcode oligonucleotide thereby forming a secondary fusion probe, b) combining the secondary fusion probe with a primary probe comprising a primary recognition domain comprising a target-binding domain; whereupon the primary-probe-binding domain binds to the primary probe; thereby forming the barcode fusion probe. In accordance with some implementations, a method of constructing a barcode fusion probe further comprises crosslinking the primary probe to the secondary fusion probe, In accordance with some implementations, a method of constructing a barcode fusion probe further comprises purifying the barcode fusion probe.

In accordance with some implementations, a barcode fusion probe comprises a barcode oligonucleotide comprising an HCR initiator or an HCR fractional initiator. In accordance with some implementations, a barcode fusion probe comprises an enzymatic conjugation domain comprising an HUH domain. In accordance with some implementations, a barcode fusion probe comprises an enzymatic conjugation domain comprising an HUH domain that is DCV.

In accordance with some implementations, a panel of N barcode fusion probes for detection of a panel of N targets is provided, wherein N is a positive integer. In some embodiments, barcode fusion probe j (for j=1, . . . , N) comprises a primary fusion probe j comprising: a) a primary recognition domain j comprising a primary antibody, an antibody fragment, a nanobody, or a polypeptide, wherein the primary antibody, antibody fragment, nanobody, or polypeptide comprises a target-binding domain j, b) an enzymatic conjugation domain j, and c) a barcode oligonucleotide j; wherein the primary recognition domain j is fused to the enzymatic conjugation domain j, and wherein the enzymatic conjugation domain j is covalently coupled to the barcode oligonucleotide j.

In accordance with some implementations, a panel of N barcode fusion probes for detection of a panel of N targets is provided, wherein N is a positive integer. In accordance with some implementations, a barcode fusion probe j (for j=1, . . . , N) comprises: a) a primary probe j comprising a primary recognition domain j comprising a primary antibody, an antibody fragment, a nanobody, or a polypeptide, wherein the primary antibody, antibody fragment, nanobody, or polypeptide comprises a target-binding domain j, b) a secondary fusion probe j comprising: i) a secondary recognition domain j comprising a secondary antibody, an antibody fragment, a nanobody, or a polypeptide comprising a primary-probe-binding domain j, ii) an enzymatic conjugation domain j, and iii) a barcode oligonucleotide j; wherein the secondary recognition domain j is fused to the enzymatic conjugation domain j, and wherein the enzymatic conjugation domain j is covalently coupled to the barcode oligonucleotide j, and wherein the secondary fusion probe j is bound to the primary probe j.

In accordance with some implementations, a panel of N barcode fusion probes for detection of a panel of N targets is provided, wherein N is a positive integer. In some embodiments, barcode oligonucleotide j comprises an HCR initiator j or an HCR fractional initiator j for j=1, . . . , N. In accordance with some implementations, a panel of N barcode fusion probes for detection of a panel of N targets is provided, wherein the same enzymatic conjugation domain is used for two or more fusion probes in the panel. In accordance with some implementations, there is a panel of N barcode fusion probes for detection of a panel of N targets wherein the same secondary recognition domain is used for two or more fusion probes in the panel. In accordance with some implementations, there is a panel of N barcode fusion probes for detection of a panel of N targets wherein target-binding domain j is specific to target j and barcode oligonucleotide j is unique to target j for j=1, . . . , N.

In accordance with some implementations, a method is provided for amplified detection of a target in a sample, the method comprising: a) providing a sample containing a target; b) providing a probe set comprising either: i) an HCR initiator-labeled fusion probe, or ii) a probe unit comprising two or more HCR fractional-initiator fusion probes; c) providing a reporter-labeled first HCR amplifier; d) generating a signal, directly or indirectly, from one or more reporter-decorated HCR amplification polymers; e) detecting the signal. In accordance with some implementations, an HCR initiator-labeled fusion probe comprises: a barcode fusion probe comprising a primary probe comprising a target-binding domain, wherein either the primary probe or a secondary probe bound to the primary probe comprises a fusion to an enzymatic conjugation domain covalently coupled to a barcode oligonucleotide comprising an HCR initiator. In accordance with some implementations, an HCR fractional-initiator fusion probe comprises: a barcode fusion probe comprising a primary probe comprising a target-binding domain, wherein either the primary probe or a secondary probe bound to the primary probe comprises a fusion to an enzymatic conjugation domain covalently coupled to a barcode oligonucleotide comprising an HCR fractional initiator. In accordance with some implementations, an HCR amplifier comprises two or more HCR hairpins. In accordance with some implementations, an HCR hairpin comprises an input domain comprising a single-stranded toehold and a stem section. In accordance with some implementations, an HCR hairpin further comprises an output domain comprising a single-stranded loop and a complement to the stem section. In accordance with some implementations, at least one HCR hairpin further comprises one or more reporters. In accordance with some implementations, when the HCR initiator-labeled probe or probe unit is bound to the target, the HCR initiator or colocalized full initiator comprising two or more HCR fractional-initiators initiates HCR signal amplification whereupon the HCR hairpins self-assemble into a tethered reporter-decorated HCR amplification polymer thereby generating a signal. In accordance with some implementations, a wash step is performed between steps b) and c). In accordance with some implementations, a wash step is performed between steps c) and d). In accordance with some implementations, the signal is removed from the sample following step e). In accordance with some implementations, the target comprises a protein, a peptide, an amino acid, a non-natural amino acid analog, a nucleic acid, a non-natural nucleic acid analog, a tag, a hapten, a fluorophore, a reporter, a chemical, a biological molecule, a pathogen, a small molecule, a macromolecule, a complex of molecules, or a set of molecules in proximity. In accordance with some implementations, a probe unit comprising two or more HCR fractional-initiator probes further comprises one or more proximity probes. In accordance with some implementations, each HCR fractional-initiator probe within a probe unit further comprises a proximity domain. In accordance with some implementations, the one or more proximity probes bind to the proximity domains within a probe unit to colocalize a full HCR initiator capable of triggering HCR signal amplification. In accordance with some implementations, any of the steps of the method are repeated to detect another target in the sample.

### BRIEF DESCRIPTION OF THE DRAWINGS

FIGS. 1A-1B depict some embodiments of barcode fusion probes.

FIGS. 2A-2D depict some embodiments of barcode fusion probes.

FIGS. 3A-3B depict some embodiments of imaging molecular targets using initiator-labeled barcode fusion probes.

FIGS. 4A-4B depict some embodiments of imaging molecular targets using initiator-labeled barcode fusion probes.

FIG. 5 demonstrates efficient covalent coupling of a fusion probe to a barcode oligonucleotide.

FIGS. 6A-6D demonstrate imaging of tagged proteins in tissue using initiator-labeled barcode fusion probes and HCR signal amplification.

FIGS. 7A-7B demonstrate imaging of a protein fusion in tissue using initiator-labeled barcode fusion probes and HCR signal amplification.

FIGS. 8A-8D demonstrate imaging of a protein target in tissue using unmodified primary antibody probes, initiator-labeled secondary barcode fusion probes, and HCR signal amplification.

FIGS. 9A-9B demonstrate imaging of a protein target in tissue using unmodified primary antibody probes, initiator-labeled secondary barcode fusion probes, and HCR signal amplification.

FIGS. 10A-10C demonstrate multiplex protein imaging in tissue using initiator-labeled barcode fusion probes and HCR signal amplification.

FIGS. 11A-11C depicts some embodiments of HCR RNA-FISH using initiator-labeled probes.

FIGS. 12A-12B depict some embodiments of HCR RNA-FISH using fractional-initiator probes.

FIGS. 13A-13D depict some embodiments of HCR IF using initiator-labeled barcode probes or barcode fusion probes.

FIGS. 13E-13F depict some embodiments of HCR RNA-FISH/IF using initiator-labeled barcode probes or barcode fusion probes for protein targets and fractional-initiator probes for RNA targets.

FIG. 14 depicts some embodiments of HCR amplification using two HCR hairpins.

FIGS. 15A-15N depict some embodiments of initiator-labeled barcode probes or barcode fusion probes.

FIGS. 16A-16D depict some embodiments of fractional-initiator barcode fusion probes colocalized by a target.

FIGS. 17A-17E depict some embodiments of fractional-initiator barcode probes or barcode fusion probes colocalized by a target complex or a set of targets in proximity.

FIGS. 18A-18R depicts some embodiments of fractional-initiator barcode probes or barcode fusion probes colocalized by a target, a target complex, or a set of targets in proximity, either direction or indirectly.

FIG. 19 depicts some embodiments of barcode probes.

FIGS. 20A-20B depict some embodiments of barcode probes.

FIGS. 21A-21B depict some embodiments of imaging molecular targets using initiator-labeled barcode probes.

FIG. 22A depicts some embodiments of detection of targets in a complex or in proximity using primary probes, fractional-initiator secondary barcode probes or barcode fusion probes, and a proximity probe.

FIG. 22B depicts some embodiments of detection of a target using primary probes, fractional-initiator secondary barcode probes or barcode fusion probes, and a proximity probe.

FIGS. 23A-23B demonstrate imaging of protein targets in cells using unmodified primary antibody probes, initiator-labeled secondary barcode probes, and HCR signal amplification.

FIGS. 24A-24C demonstrate multiplex protein imaging in cells using initiator-labeled barcode probes and HCR signal amplification.

### DETAILED DESCRIPTION

A molecular target can be detected in a sample using an antibody probe conjugated to a barcode oligonucleotide that mediates generation of a signal. For example, the barcode oligonucleotide can comprise an initiator capable of triggering generation of an amplified signal via the mechanism of hybridization chain reaction (HCR) 1. For a multiplex assay in which N different targets are detected in the sample simultaneously (N is a positive integer greater than 1), each different target can be detected using a different antibody probe specific to that target, carrying a barcode oligonucleotide specific to that probe, mediating the generation of a distinguishable signal specific to that barcode oligonucleotide (for example, target j detected with antibody j conjugated to barcode oligonucleotide j, generating distinguishable signal j, for j=1, . . . , N). Conjugation of a barcode oligonucleotide to a primary antibody can interfere with binding between the antibody and the target, with the degree of interference being case-specific for each antibody/target pair, as well as depending on the method used to conjugate the oligonucleotide to the antibody.

In many cases, it is challenging to conjugate an oligonucleotide to a primary antibody and retain the ability to efficiently bind the target. To overcome this difficulty, a common approach is to use an unmodified primary antibody probe to bind the target, and then to use an oligonucleotide-conjugated secondary antibody probe to bind the primary antibody probe. The benefit of this approach is that a primary antibody probe specific to a new target can be used without modification, eliminating the need to validate oligonucleotide conjugation for each new primary antibody. As a result, a large library of unmodified primary antibody probes can be used in combination with a small library of pre-validated oligonucleotide-conjugated secondary antibody probes, making it more straightforward to exploit new primary antibody probes. A downside of this approach is that each secondary antibody probe must be specific for the host organism (or isotype) of the primary antibody probe. Hence, for multiplex assays, each primary antibody must be raised in a different host organism (or be of a different isotype) so that it can be recognized by a different oligonucleotide-conjugated secondary antibody and generate a distinguishable signal specific to that target (for example, target j detected with unmodified primary antibody j generated in host j, detected with secondary antibody j specific to host j and conjugated to barcode oligonucleotide j, generating distinguishable signal j, for j=1, . . . , N). As a result, use of secondary antibody probes can restrict the degree of multiplexing if a researcher has access to libraries of primary antibody probes raised in only a small number of hosts (e.g., rabbit and mouse; enabling 2-plex target detection).

Use of oligonucleotide-conjugated primary antibody probes is limiting due to the unpredictable consequences of conjugation upon antibody affinity and specificity, but attractive for multiplexing because this approach places no restriction on the host organism (or isotype) for each primary antibody (e.g., all primary antibodies can be raised in the same host organism if convenient). Alternatively, use of oligonucleotide-conjugated secondary antibody probes avoids the need to conjugate an oligonucleotide to each new primary antibody, but is limiting for multiplexing due to the need for each primary antibody to be raised in a different host organism (or be a different isotype). There is currently a need for an approach that works robustly for different primary antibodies (to enable detection of diverse targets without requiring extensive debugging to generate a new antibody probe for each new target) and allows primary antibodies for different targets to be raised in the same host organism (to enable straightforward multiplexing). The same arguments apply if the primary probe and/or the secondary probe is an antibody fragment, a nanobody, a polypeptide, or another probe composition.

### Barcode Probes

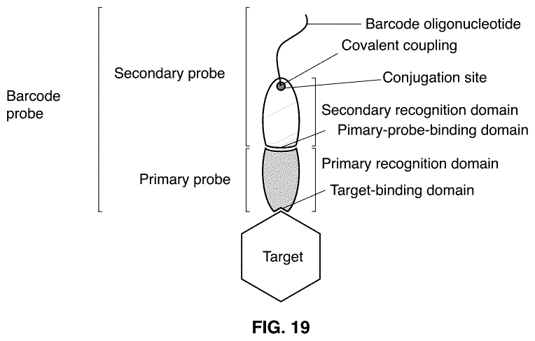

In some embodiments, a target is detected using a barcode probe (for example, FIGS. 19-21, 23 and 24).

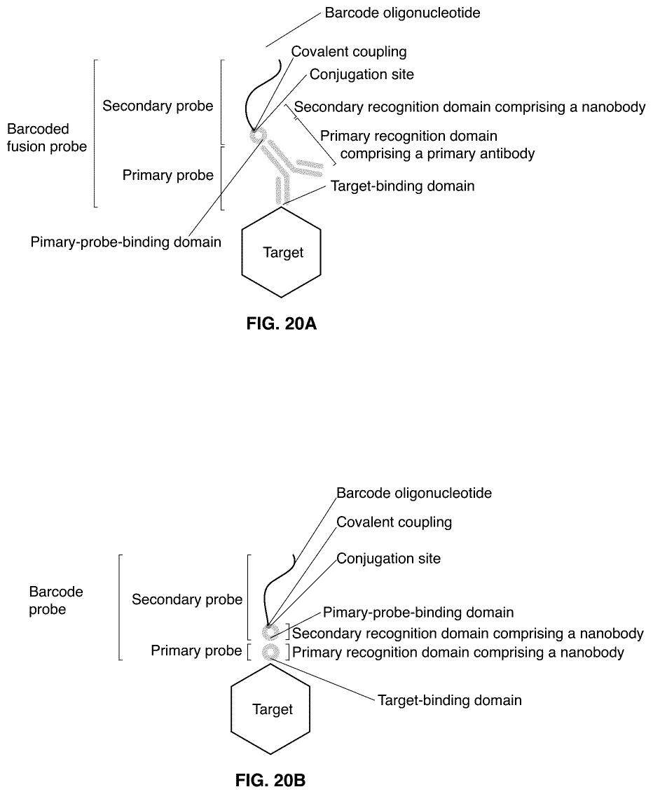

In some embodiments, a barcode probe comprises a primary probe comprising a primary recognition domain comprising a target-binding domain and further comprises a secondary probe comprising a barcode oligonucleotide and a secondary recognition domain comprising a primary-probe-binding domain and a conjugation site, wherein the conjugation site is covalently coupled to the barcode oligonucleotide and wherein the secondary probe is bound to the primary probe. In some embodiments, the secondary probe comprises two or more conjugation sites. In some embodiments, the primary probe is covalently crosslinked to the secondary probe. In some embodiments, the primary recognition domain comprises a primary antibody, an antibody fragment, a nanobody, a polypeptide, or another composition (for example, FIGS. 20A-20B). In some embodiments, the secondary recognition domain comprises an antibody, an antibody fragment, a nanobody, a polypeptide, or another composition (for example, FIGS. 20A-20B).

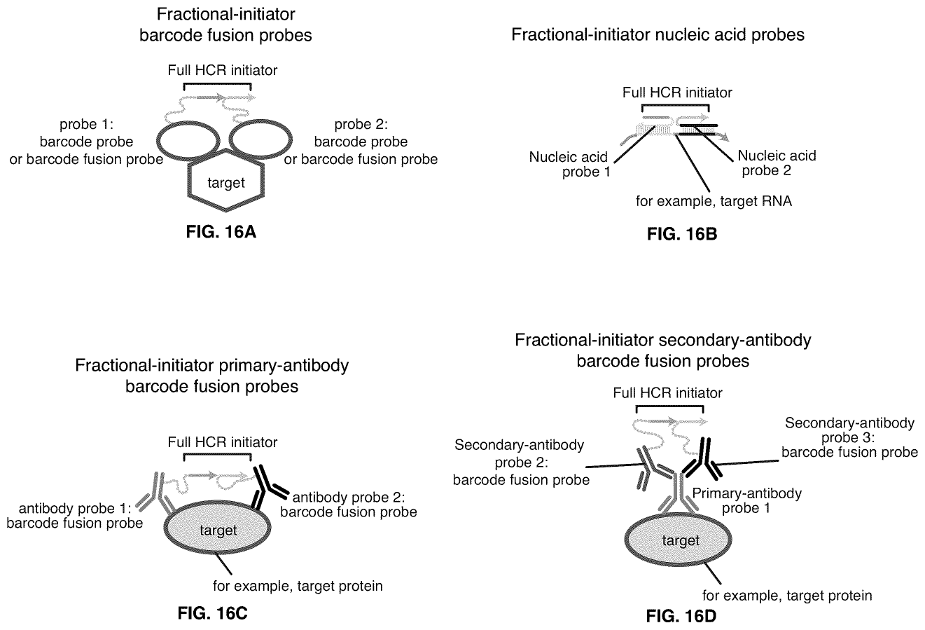

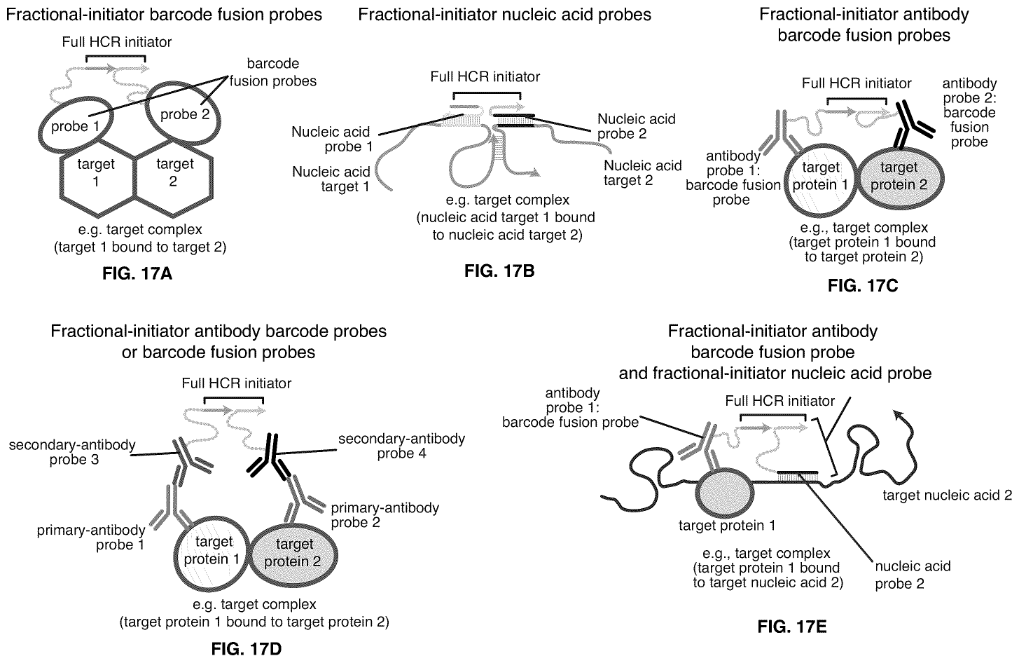

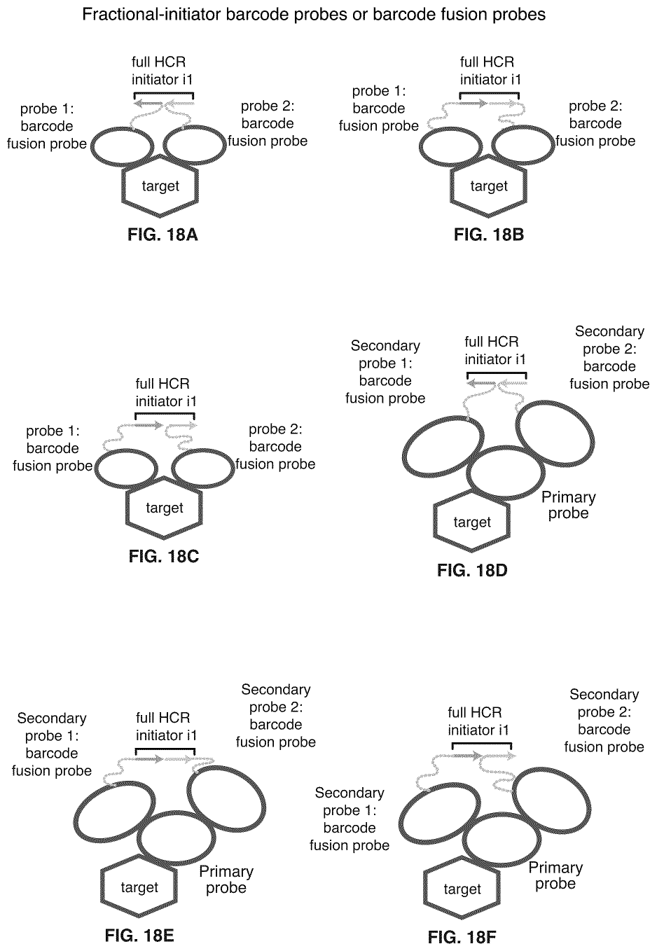

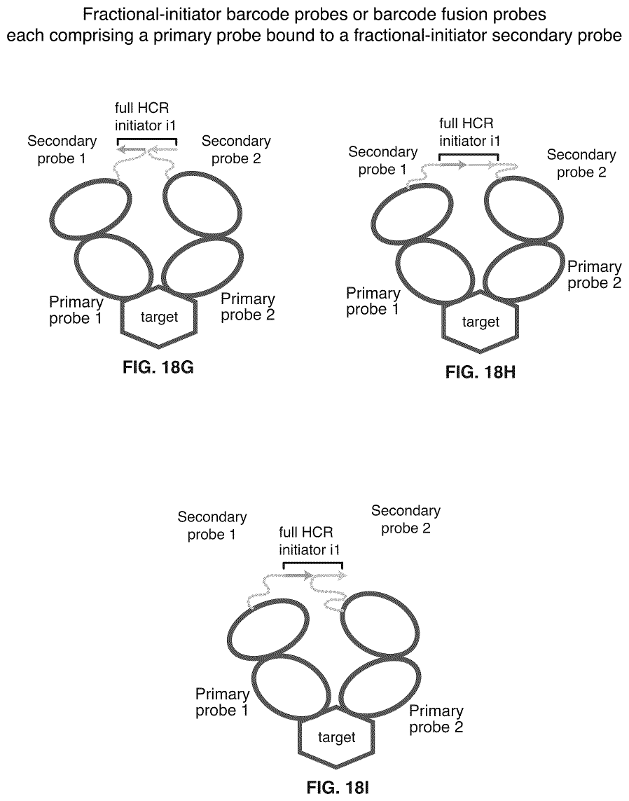

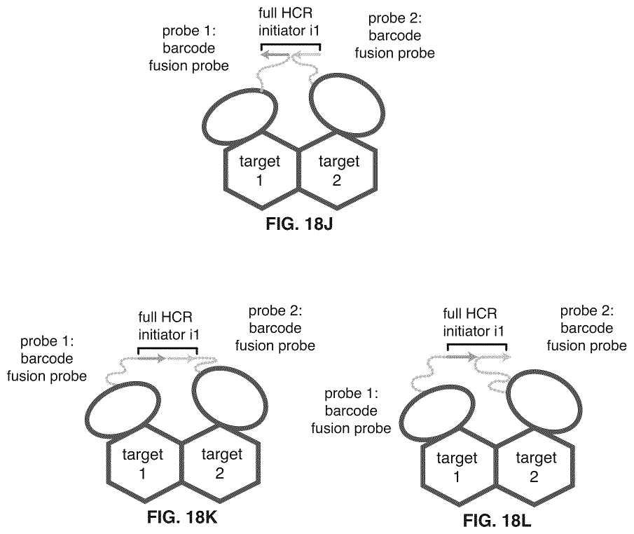

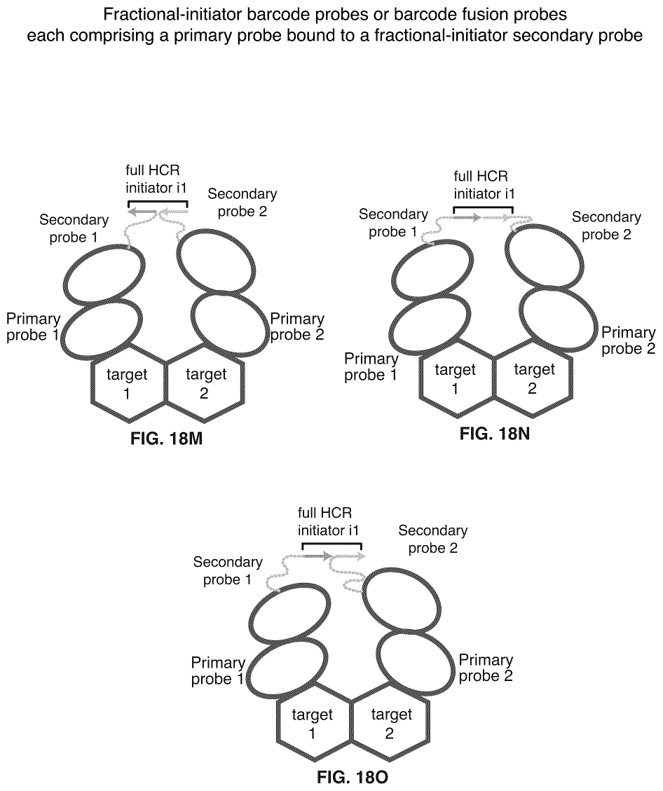

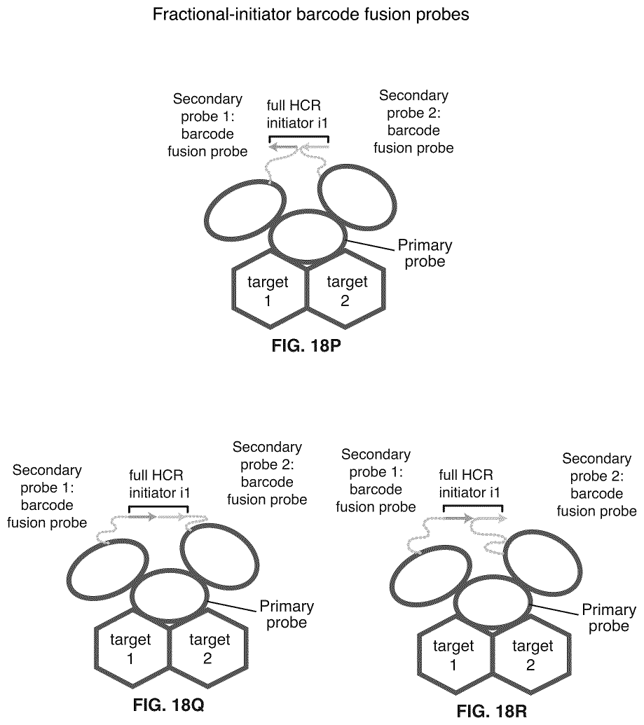

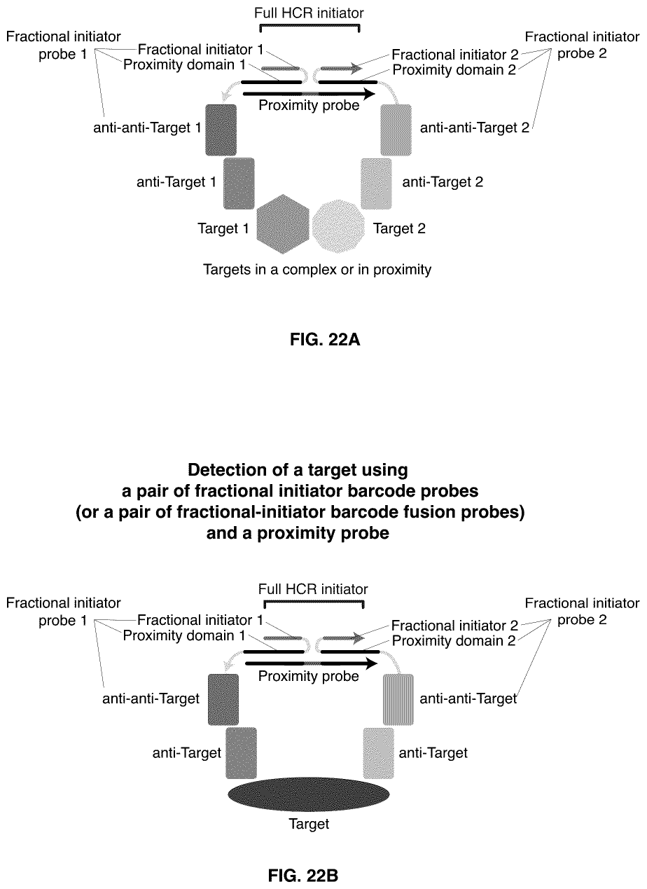

In some embodiments, the barcode probe is used to mediate generation of a signal either directly or indirectly. In some embodiments, the barcode probe comprises a barcode oligonucleotide. In some embodiments, the barcode oligonucleotide comprises an HCR initiator capable of triggering HCR signal amplification. In some embodiments, a barcode probe comprising a barcode oligonucleotide comprising an HCR initiator is also known as an initiator-labeled barcode probe or an initiator-labeled probe. In some embodiments, the barcode oligonucleotide comprises an HCR fractional initiator incapable of triggering HCR signal amplification in isolation, but capable of triggering HCR signal amplification when colocalized with one or more other HCR fractional initiators to form a full HCR initiator (for example, FIGS. 16A-16D, 17A-17E, 18A-18R, 22A-22B); in some embodiments, colocalization of two or more fractional initiators to form a full HCR initiator is mediated by one or more proximity probes (for example, FIGS. 22A-22B). In some embodiments, a barcode probe comprising a barcode oligonucleotide comprising an HCR fractional initiator is also known as a fractional-initiator barcode probe or a fractional-initiator probe.

In some embodiments, the secondary probe binds tightly to the primary probe so that binding of an individual secondary probe molecule to a cognate primary probe is approximately irreversible (that is, once the secondary probe binds to a cognate primary probe molecule it does not appreciably swap binding partners by unbinding the primary probe molecule to rebind a different primary probe molecule). In some embodiments, once a barcode probe comprising a complex of a primary probe and a secondary probe is assembled, the barcode probe remains intact even in the presence of other barcode probes.

In some embodiments, a multiplex assay in which N targets are detected simultaneously (where N is a positive integer greater than 1), can be performed using N barcode probes, where barcode probe j comprises: a primary probe j comprising a target-binding domain for target j, a secondary probe j comprising a primary-probe-binding domain for primary probe j, and a barcode oligonucleotide j. In some embodiments, barcode oligonucleotide j is an HCR initiator j capable of triggering HCR signal amplification using HCR amplifier j comprising reporter j. In some embodiments, barcode oligonucleotide j is an HCR fractional initiator j incapable of triggering HCR signal amplification on its own, but when colocalized with one or more other fractional initiators to form a full HCR initiator j (optionally mediated by one or more proximity probes j), is capable of triggering HCR signal amplification using HCR amplifier j comprising reporter j.

### Barcode Fusion Probes

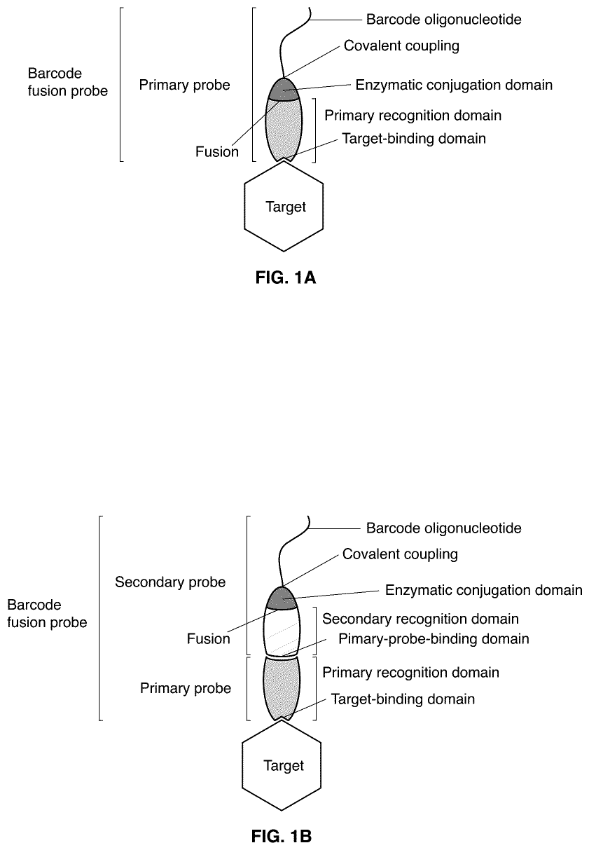

In some embodiments, a target is detected using a barcode fusion probe (for example, FIGS. 1-4, and 6-10).

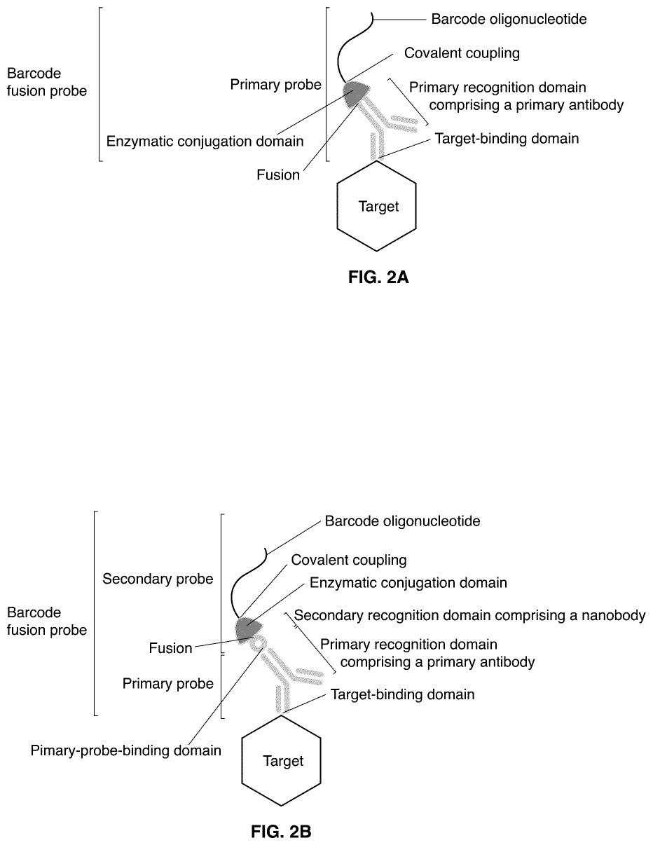

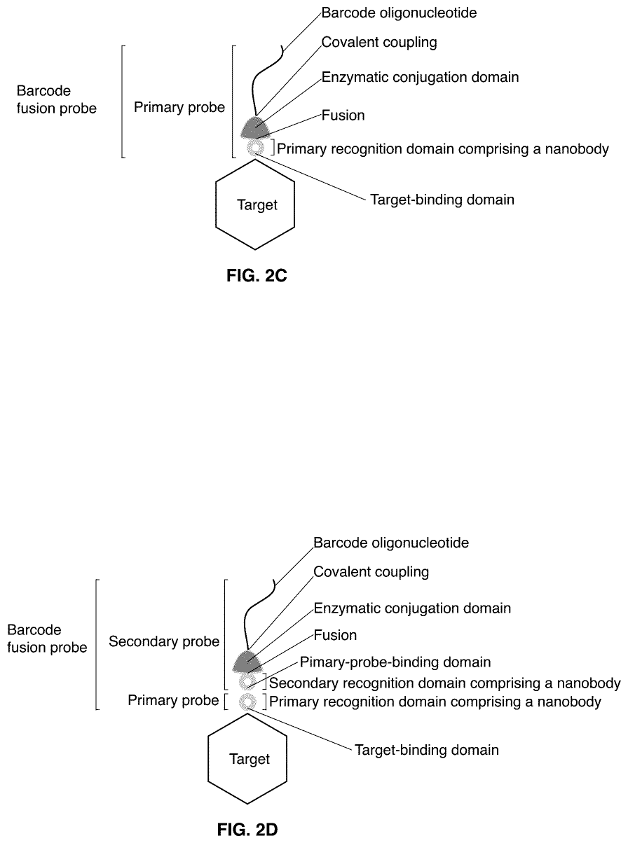

In some embodiments, the barcode fusion probe comprises a primary probe comprising a primary recognition domain comprising a target-binding domain and further comprises a secondary probe comprising a barcode oligonucleotide and a fusion of an enzymatic conjugation domain and a secondary recognition domain comprising a primary-probe-binding domain, wherein the enzymatic conjugation domain is covalently coupled to the barcode oligonucleotide and wherein the secondary probe is bound to the primary probe. In some embodiments, the primary probe is covalently crosslinked to the secondary probe. In some embodiments, the primary recognition domain comprises a primary antibody, an antibody fragment, a nanobody, a polypeptide, or another composition (for example, FIGS. 1B, 2B, and 2D). In some embodiments, the secondary recognition domain comprises an antibody, an antibody fragment, a nanobody, a polypeptide, or another composition (for example, FIGS. 1B, 2B, and 2D).

In some embodiments, a target is detected using a barcode fusion probe (for example, FIGS. 1-3). In some embodiments, the barcode fusion probe comprises a primary probe comprising a barcode oligonucleotide and a fusion of an enzymatic conjugation domain and a primary recognition domain comprising a target-binding domain, wherein the enzymatic conjugation domain is covalently coupled to the barcode oligonucleotide. In some embodiments, the primary recognition domain comprises a primary antibody, an antibody fragment, a nanobody, a polypeptide, or another composition (for example, FIGS. 1A, 2A, and 2C).

In some embodiments, the enzymatic conjugation domain is capable of covalently coupling itself to the barcode oligonucleotide. In some embodiments, the enzymatic conjugation domain comprises an HUH domain. In some embodiments, the HUH domain is DCV.

In some embodiments, the barcode fusion probe is used to mediate generation of a signal either directly or indirectly. In some embodiments, the barcode fusion probe comprises a barcode oligonucleotide. In some embodiments, the barcode oligonucleotide comprises an HCR initiator capable of triggering HCR signal amplification. In some embodiments, a barcode fusion probe comprising a barcode oligonucleotide comprising an HCR initiator is also known as an initiator-labeled barcode fusion probe or an initiator-labeled probe. In some embodiments, the barcode oligonucleotide comprises an HCR fractional initiator incapable of triggering HCR signal amplification in isolation, but capable of triggering HCR signal amplification when colocalized with one or more other HCR fractional initiators to form a full HCR initiator (for example, FIGS. 16A-16D, 17A-17E, 18A-18R, 22A-22B); in some embodiments, colocalization of two or more fractional initiators to form a full HCR initiator is mediated by one or more proximity probes (for example, FIGS. 22A-22B). In some embodiments, a barcode fusion probe comprising a barcode oligonucleotide comprising an HCR fractional initiator is also known as a fractional-initiator barcode fusion probe or a fractional-initiator probe.

In some embodiments, the secondary probe binds tightly to the primary probe so that binding of an individual secondary probe molecule to a cognate primary probe is approximately irreversible (that is, once the secondary probe binds to a cognate primary probe molecule it does not appreciably swap binding partners by unbinding the primary probe molecule to rebind a different primary probe molecule). In some embodiments, once a barcode fusion probe comprising a complex of a primary probe and a secondary probe is assembled, the barcode fusion probe remains intact even in the presence of other barcode fusion probes.

In some embodiments, a multiplex assay in which N targets are detected simultaneously (N is a positive integer greater than 1), can be performed using N barcode fusion probes, where barcode fusion probe j comprises: a primary probe j comprising a target-binding domain for target j, a secondary probe j comprising a primary-probe-binding domain for primary probe j and a barcode oligonucleotide j. In some embodiments, barcode oligonucleotide j is an HCR initiator j capable of triggering HCR signal amplification using HCR amplifier j comprising reporter j. In some embodiments, barcode oligonucleotide j is an HCR fractional initiator j incapable of triggering HCR signal amplification on its own, but when colocalized with one or more other fractional initiators to form a full HCR initiator j (optionally mediated by one or more proximity probes j), is capable of triggering HCR signal amplification using HCR amplifier j comprising reporter j.

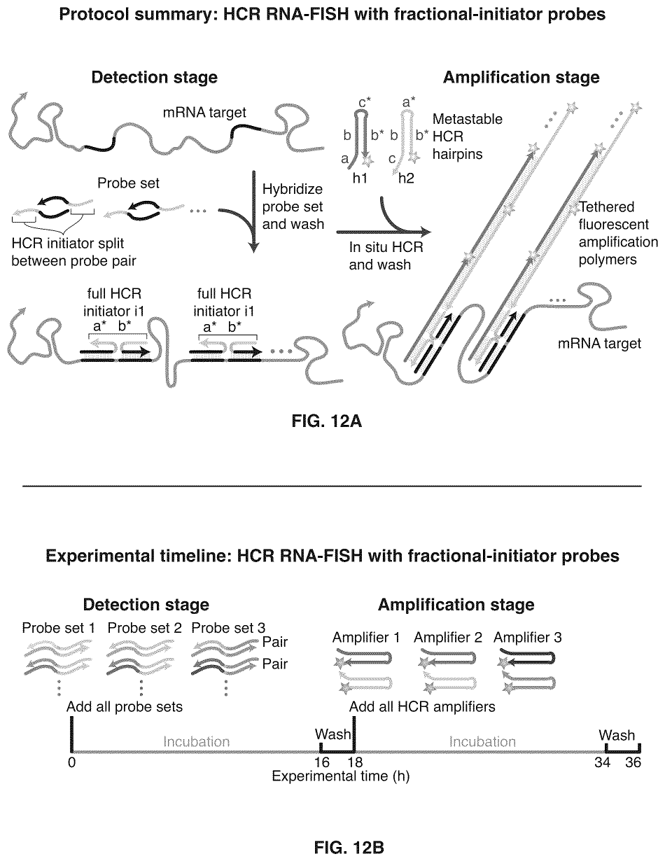

In some embodiments, a target is detected within a sample using a probe set comprising one or more probe units (for example see the probe sets of FIGS. 12A-12B and 22A-22B), where a probe unit comprises two or more fractional-initiator barcode probes (also known as fractional-initiator probes) or two or more fractional-initiator barcode fusion probes (also known as fractional-initiator probes) (for example see the probe units of FIGS. 16A-16D, 17A-17E, 18A-18R, and 22A-22B), where each fractional-initiator barcode probe or fractional-initiator barcode fusion probe comprises a target-binding domain and a barcode oligonucleotide comprising a fractional initiator. In some embodiments, binding of each probe within a probe unit to proximal cognate binding sites on the target colocalizes the fractional initiators to form a full HCR initiator (for example, see the full HCR initiators of FIGS. 12A, 16A-16D, 17A-17E, 18A-18R, and 22A-22B) capable of triggering HCR signal amplification. In some embodiments, each fractional-initiator probe within a probe unit further comprises a proximity domain (for example, see FIGS. 22A-22B). In some embodiments, binding of each fractional-initiator probe within a probe unit to cognate binding sites on proximal targets enables binding of one or more proximity probes to the proximity domains within the probe unit to colocalize the fractional initiators to form a colocalized full HCR initiator (for example, see FIGS. 22A-22B) capable of triggering HCR signal amplification.

In some embodiments, a multiplex assay in which N targets are detected simultaneously (N a positive integer greater than 1), can be performed using N barcode fusion probes, where barcode fusion probe j comprises: a primary probe j comprising a target-binding domain for target j and a barcode oligonucleotide j. In some embodiments, barcode oligonucleotide j is an HCR initiator j capable of triggering HCR signal amplification using HCR amplifier j comprising distinguishable reporter j. In some embodiments, barcode oligonucleotide j is an HCR fractional initiator j incapable of triggering HCR signal amplification on its own, but when colocalized with one or more other fractional initiators to form a full HCR initiator j (optionally mediated by one or more proximity probes j), is capable of triggering HCR signal amplification using HCR amplifier j comprising reporter j.

### Primary Probe

In some embodiments, a primary probe comprises a primary recognition domain comprising an antibody, an antibody fragment, a nanobody, a polypeptide, or another composition wherein the antibody, antibody fragment, nanobody, polypeptide, or other composition comprises a target-binding domain (for example, see FIGS. 1AB, 2A-2D, 19, 20A-20B). In some embodiments, the primary recognition domain comprises a primary antibody. In some embodiments, the primary probe comprises an amino acid, a synthetic amino acid analog, DNA, RNA, a synthetic nucleic acid analog, a chemical, a linker, streptavidin, biotin, DIG, a hapten, and/or any combination of the above comprising a target-binding domain. In some embodiments, the primary probe comprises a molecule or a complex of molecules comprising a target-binding domain. In some embodiments of barcode fusion probes, the primary probe comprises an enzymatic conjugation domain wherein the primary recognition domain is fused to the enzymatic conjugation domain and wherein the enzymatic conjugation domain is capable of covalently coupling to a barcode oligonucleotide. In some embodiments, the primary probe comprises a barcode oligonucleotide covalently coupled to the enzymatic conjugation domain (for example, FIGS. 1A, 2A, and 2C). In some embodiments, the primary probe is associated, directly or indirectly, with a barcode oligonucleotide.

### Secondary Probe

In some embodiments, a secondary probe comprises a secondary recognition domain comprising an antibody, an antibody fragment, a nanobody, a polypeptide, or another composition, wherein the antibody, antibody fragment, nanobody, polypeptide, or other composition comprises a primary-probe-binding domain (for examples, see FIGS. 1B, 2B, 2D, 19, 20A-20B). In some embodiments, the primary-probe-binding domain binds to the primary probe. In some embodiments, the secondary probe recognition domain comprises a protein fragment comprising a primary-probe-binding domain (for example, an Fc-binding domain from Protein A or an Fc-binding domain from Protein G). In some embodiments, the secondary recognition domain comprises an aptamer comprising a primary-probe-binding domain. In some embodiments, the secondary recognition domain comprises a primary antibody or a secondary antibody. In some embodiments, the secondary recognition domain comprises a nanobody. In some embodiments, the secondary probe comprises an amino acid, a synthetic amino acid analog, DNA, RNA, a synthetic nucleic acid analog, a chemical a linker, streptavidin, biotin, DIG, a hapten, and/or any combination of the above comprising a primary-probe-binding domain. In some embodiments, the secondary probe comprises a molecule or complex of molecules comprising a primary-probe-binding domain.

In some embodiments of barcode probes, the secondary probe comprises a conjugation site. In some embodiments, the secondary probe comprises a barcode oligonucleotide covalently coupled to the conjugation site (for example, FIGS. 19, 20A-20B). In some embodiments, the secondary probe comprises two or more conjugation sites.

In some embodiments of barcode fusion probes, the secondary probe comprises an enzymatic conjugation domain wherein the secondary recognition domain is fused to the enzymatic conjugation domain and wherein the enzymatic conjugation domain is capable of coupling to a barcode oligonucleotide. In some embodiments, the secondary probe comprises a barcode oligonucleotide covalently coupled to the enzymatic conjugation domain (for example, FIGS. 1A, 2B, and 2D).

In some embodiments, the secondary probe is associated, directly or indirectly, with a barcode oligonucleotide. In some embodiments, the secondary recognition domain comprises one of the following nanobodies: NbALFA (binds to ALFA tag), NbVHH05 (binds to VHH05 tag), Nb127D01 (binds to 127bind to rabbit IgG), NbGFP (binds to GFP), TP1106 (binds to Mouse Fab, IgG1/2a/2b-kappa), TP1107 (binds to mouse Fc, IgG1), TP1170 (binds to mouse Ig, kappa), TP1129 (binds to mouse IgG2a), TP897 (binds to rabbit IgG).

### Covalent Coupling of the Conjugation Site to the Barcode Oligonucleotide for Barcode Probes

In some embodiments of barcode probes, a conjugation site is covalently coupled to a barcode oligonucleotide.

In some embodiments, the conjugation site comprises naturally occurring or synthetically engineered lysine residues. In some embodiments, the barcode oligonucleotide comprises an amine modification. In some embodiments, the probe comprising the conjugation site and the barcode oligonucleotide can be reacted with an NHS ester to form a stable covalent conjugate.² In some embodiments, the probe comprising the conjugation site and the barcode oligonucleotide can be reacted with S-HyNic and Sulfo-S-4FB to form a stable covalent conjugate.²

In some embodiments, the conjugation site comprises naturally occurring or synthetically engineered cysteine residues. In some embodiments, the barcode oligonucleotide comprises a thiol-reactive group which will react with thiol groups on proteins to form thioether-coupled stable covalent bonds. In some embodiments, the barcode oligonucleotide comprises a maleimide, maleimide derivatives or maleimide precursor.³ In some embodiments, the barcode oligonucleotide comprises activated alkyl halides such as iodoacetamide. In some embodiments, the barcode oligonucleotide comprises other reactive electrophilic reagents such as disulfides, perfluoroarene, or Michael acceptors.⁴ In some embodiments, the probe comprising the conjugation site and the barcode oligonucleotide can be coupled by linker pairs: one linker modifies and couples to the probe, and the other linker modifies and couples to the oligonucleotide. The linker pairs can chemically couple to each other. In some embodiments for coupling amine-containing oligo and cysteine containing probe, the linker pairs are 6-Azidohexanoic Acid Ester and Alkyne-PEG4 (or DBCO)-Maleimide.

In some embodiments, the probe comprising the conjugation site and the barcode oligonucleotide can be reacted with a reactive disulfide intermediate to form a stable covalent conjugate.³ In some embodiments, a conjugation site comprising an N-terminal cysteine residue and the barcode oligonucleotide can undergo native chemical ligation to form a stable covalent conjugate.⁵

In some embodiments, the conjugation site comprises unnatural amino acids or reactive azide modifications. In some embodiments, the barcode oligonucleotide comprises an alkyne modification or a dibenzocyclooctyl modification. In some embodiments, the conjugation site and the barcode oligonucleotide can be reacted with or without a copper catalyst to form a stable covalent conjugate.^{6,7}

In some embodiments, the conjugation site comprises a ketone. In some embodiments, the barcode oligonucleotide comprises an aminooxy modification. In some embodiments, the conjugation site and the barcode oligonucleotide can be reacted to form a stable covalent conjugate.⁸

In some embodiments, the conjugation site comprises a tetrazine. In some embodiments, the barcode oligonucleotide comprises a trans-cyclooctene. In some embodiments, the conjugation site and the barcode oligonucleotide can be reacted to form a stable covalent conjugate.⁹

In some embodiments, the conjugation site is covalently coupled to the barcode oligonucleotide via a light-activated reaction, a chemical reaction, a catalyzed reaction, or an enzymatic reaction.

In some embodiments, the conjugation site and the barcode oligonucleotide can be reacted with dithiothreitol under UV irradiation to form a stable covalent conjugate.¹⁰ In some embodiments, the conjugation site comprises benzoyl-phenylalanine. In some embodiments, the conjugation site and the barcode oligonucleotide can be reacted under UV irradiation to form a stable covalent conjugate.¹¹

In some embodiments of barcode probes, the conjugation site is capable of covalently coupling to a barcode oligonucleotide through an enzyme. In some embodiments, the conjugation site comprises a coupling recognition domain comprising a specific sequence. In some embodiments, the barcode oligonucleotide comprises a molecular tag. In some embodiments, the conjugation enzyme is sortase A, asparaginyl endopeptidase, formylglycine generating enzyme, or protein farnesyl transferase. In some embodiments, the conjugation site and the barcode oligonucleotide can be reacted with an enzyme to form a stable covalent conjugate.¹²

### Covalent Coupling of the Enzymatic Conjugation Domain to the Barcode Oligonucleotide for Barcode Fusion Probes

In some embodiments of barcode fusion probes, the enzymatic conjugation domain is capable of covalently coupling itself to a barcode oligonucleotide. In some embodiments, the enzymatic conjugation domain is capable of covalently coupling itself to a barcode oligonucleotide comprising a coupling recognition domain comprising a specific sequence. In some embodiments, the enzymatic conjugation domain is an HUH domain.¹³ In some embodiments, the HUH domain is DCV, PCV2, FBNYV, RepB, TraI, NES, mMobA, or RepBm.¹³ In some embodiments, the enzymatic conjugation domain is a HaloTag or SnapTag.^{14,15}

### Barcode Oligonucleotide

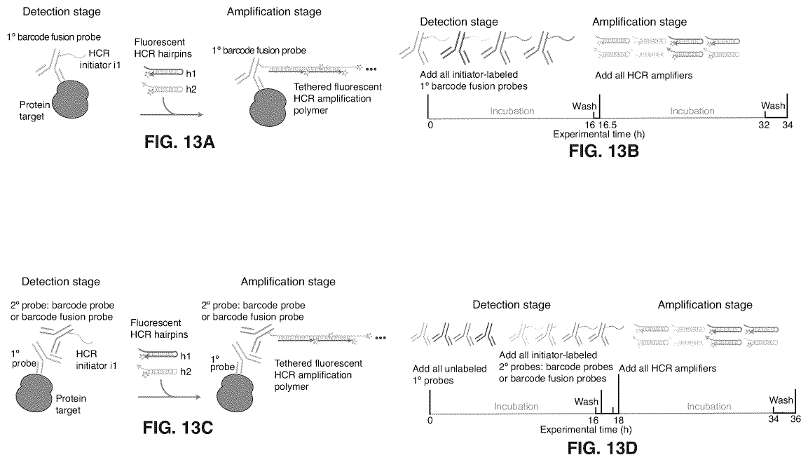

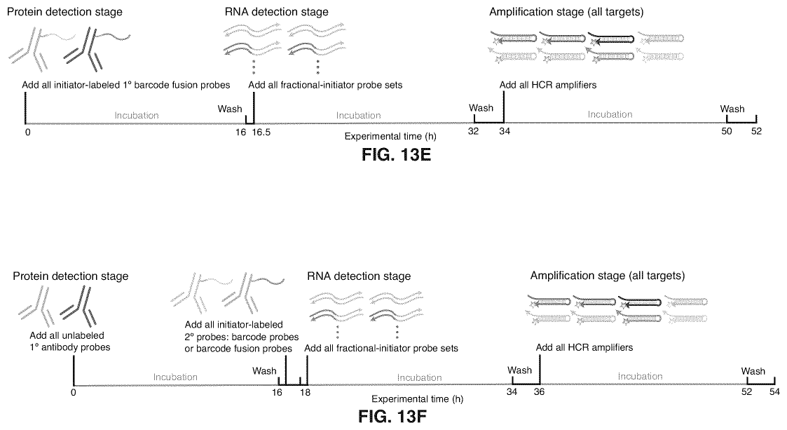

In some embodiments the barcode oligonucleotide comprises DNA, RNA, a synthetic nucleic acid analog, an amino acid, a peptide, a synthetic amino acid analog, a hapten, a tag, a chemical linker, and/or any combination of the above. In some embodiments, the barcode oligonucleotide comprises a barcode domain that facilitates generation of a signal directly or indirectly by initiating a chain reaction in which reporter-labeled HCR hairpins self-assemble into tethered reporter-decorated HCR amplification polymers, generating an amplified signal at the site of the target within a sample (for example, FIGS. 13A, 13C). In some embodiments, the barcode oligonucleotide comprises an optional linker domain between the conjugation site and the barcode domain.

In some embodiments, the barcode domain comprises an HCR initiator. In some embodiments, the barcode domain comprises an HCR fractional initiator. In some embodiments, a barcode probe comprising a barcode domain comprising an HCR initiator is also known as an initiator-labeled barcode probe or an initiator-labeled probe. In some embodiments, a barcode probe comprising a barcode oligonucleotide comprising an HCR fractional initiator is also known as a fractional-initiator barcode probe or a fractional-initiator probe. In some embodiments, a barcode fusion probe comprising a barcode domain comprising an HCR initiator is also known as an initiator-labeled barcode fusion probe or an initiator-labeled probe. In some embodiments, a barcode fusion probe comprising a barcode oligonucleotide comprising an HCR fractional initiator is also known as a fractional-initiator barcode fusion probe or a fractional-initiator probe.

In some embodiments of barcode fusion probes, the barcode oligonucleotide comprises a coupling recognition domain comprising a specific sequence that facilitates enzymatic coupling to an enzymatic conjugation domain. In some embodiments, the barcode oligonucleotide comprises an optional linker domain between the coupling recognition domain and the barcode domain.

In some embodiments, the barcode oligonucleotide comprises 5, 10, 20, 30, 40, 50, 60, 70, 80, 90, or 100 nucleotides or any intermediate number of nucleotides. In some embodiments, the barcode oligonucleotide comprises 5, 10, 20, 50, 100, 200, 500, or 1000 nucleotides or any intermediate number of nucleotides.

### Assembly of a Barcode Probe Comprising a Primary Probe and a Secondary Probe

In some embodiments, the process of assembling the barcode probe comprises some or all of the following steps. In some embodiments, the process of assembling the barcode probe comprises at least steps 1 and 3, at least steps 1-3, at least steps 1-4, at least steps 1-5 or at least steps 1-6. In some embodiments, the steps are performed in numerical order.
Step 1: create a secondary probe by covalently coupling a barcode oligonucleotide to a conjugation site on a secondary recognition domain comprising a primary-probe-binding domain and a conjugation site
Step 2: optionally purifying the product of Step 1
Step 3: combining the secondary probe with a primary probe comprising a primary recognition domain comprising a target-binding domain; whereupon the primary-probe-binding domain binds to the primary probe to create a barcode probe
Step 4: optionally purifying the product of Step 3
Step 5: optionally crosslinking the primary probe to the secondary probe (for example, via chemical and/or light-activated fixation or crosslinking)
Step 6: optionally purifying the product of Step 5

Any subset of the above steps can be performed in any order any number of times.

In some embodiments, the secondary recognition domain comprising a conjugation site and a primary-probe-binding domain is expressed and optionally purified prior to Step 1.

In some embodiments, the process of assembling the barcode probe comprises some or all of the following steps. In some embodiments, the process of assembling the barcode probe comprises at least steps 1 and 5, at least steps 1-5, or at least steps 1-6. In some embodiments, the steps are performed in numerical order.
Step 1: combining i) a secondary recognition domain comprising a primary-probe-binding domain and a conjugation site with ii) a primary probe comprising a primary recognition domain comprising a target-binding domain; whereupon the primary-probe-binding domain binds to the primary probe
Step 2: optionally purifying the product of Step 1
Step 3: optionally crosslinking the primary probe to secondary recognition domain (for example, via chemical and/or light-activated fixation or crosslinking)
Step 4: optionally purifying the product of Step 3
Step 5: covalently coupling a barcode oligonucleotide to the conjugation site on the product of Step 1 or Step 3 to create a barcode probe
Step 6: optionally purifying the product of Step 5

Any subset of the above steps can be performed in any order any number of times. In some embodiments, the barcode oligonucleotide is combined with the product of Step 1 or Step 3 in excess, or vice versa. In some embodiments, the secondary recognition domain is combined with the primary probe with the primary probe in excess, or vice versa. In some embodiments, the secondary recognition domain comprising a conjugation site and a primary-probe-binding domain is expressed and optionally purified prior to Step 1.

In some embodiments, the process of assembling the barcode probe comprises some or all of the following steps. In some embodiments, the process of assembling the barcode probe comprises at least step 1. In some embodiments, the process of assembling the barcode probe comprises at least step 2. In some embodiments, the process of assembling the barcode probe comprises at least steps 1-2. In some embodiments, the steps are performed in numerical order.

Step 1: combining a barcode oligonucleotide with a secondary recognition domain comprising a primary-probe-binding domain and a conjugation site and covalently coupling the conjugation site to the barcode oligonucleotide, thereby forming a secondary probe.

Step 2: combining the secondary probe with a primary probe comprising a primary recognition domain comprising a target-binding domain; whereupon the primary-probe-binding domain binds to the primary probe, thereby forming the barcode probe.

In some embodiments, a barcode probe is assembled from the following ingredients: (1) a primary probe comprising a primary recognition domain comprising a target-binding domain, (2) a secondary recognition domain comprising a primary-probe binding domain and a conjugation site, (3) a barcode oligonucleotide. In some embodiments, a barcode probe is assembled using a method comprising some or all of the following steps, performed in any order, any number of times: (step 1) chemically modifying a barcode oligonucleotide, (step 2) chemically modifying the conjugation site within the secondary recognition domain, (step 3) combining the barcode oligonucleotide and the conjugation site and optionally a catalyst or enzyme to covalently couple the barcode oligonucleotide to the conjugation site to create a secondary probe, (step 4) combining the secondary probe with the primary probe to create a barcode probe, (step 5) expressing the primary probe, (step 6) expressing the secondary recognition domain, (step 7) purify the product of any other step, (step 8) covalently crosslinking the primary probe to the secondary probe. In some embodiments, some of the steps can be omitted. In some embodiments, the method of assembling the barcode probe comprises a subset of the above steps performed in any order any number of times. In some embodiments, the steps are performed in numerical order. In some embodiments, the barcode oligonucleotide is combined with the conjugation site in excess, or vice versa. In some embodiments, the primary probe is combined with the secondary probe in excess, or vice versa. In some embodiments, the secondary recognition domain is combined with the primary probe prior to conjugation of the barcode oligonucleotide to the conjugation site on the secondary recognition domain, or vice versa.

### Assembly of a Barcode Fusion Probe Comprising a Primary Probe and a Secondary Probe

In some embodiments, the process of assembling the barcode fusion probe comprises some or all of the following steps. In some embodiments, the process of assembling the barcode fusion probe comprises at least steps 1 and 3, at least steps 1-3, at least steps 1-4, at least steps 1-5 or at least steps 1-6. In some embodiments, the steps are performed in numerical order.
Step 1: combining the barcode oligonucleotide with a fusion comprising: a) a secondary recognition domain comprising a primary-probe-binding domain, and b) an enzymatic conjugation domain; whereupon the enzymatic conjugation domain covalently couples to the barcode oligonucleotide to create a secondary probe
Step 2: optionally purifying the product of Step 1
Step 3: combining the secondary probe with a primary probe comprising a primary recognition domain comprising a target-binding domain; whereupon the primary-probe-binding domain binds to the primary probe to create a barcode fusion probe
Step 4: optionally purifying the product of Step 3
Step 5: optionally crosslinking the primary probe to the secondary probe (for example, via chemical and/or light-activated fixation or crosslinking)
Step 6: optionally purifying the product of Step 5

Any subset of the above steps can be performed in any order any number of times. In some embodiments, the fusion is expressed and optionally purified prior to Step 1.

In some embodiments, the process of assembling the barcode fusion probe comprises some or all of the following steps. In some embodiments, the process of assembling the barcode fusion probe comprises at least steps 1 and 5, at least steps 1-5, or steps 1-6. In some embodiments, the steps are performed in numerical order.
Step 1: combining a fusion comprising: a) a secondary recognition domain comprising a primary-probe-binding domain, and b) an enzymatic conjugation domain, with a primary probe comprising a primary recognition domain comprising a target-binding domain; whereupon the primary-probe-binding domain binds to the primary probe
Step 2: optionally purifying the product of Step 1
Step 3: optionally crosslinking the primary probe to the fusion (for example, via chemical and/or light-activated fixation or crosslinking)
Step 4: optionally purifying the product of Step 3
Step 5: combining the product of Step 1 or Step 3 with the barcode oligonucleotide, whereupon the enzymatic conjugation domain covalently couples to the barcode oligonucleotide to create a barcode fusion probe
Step 6: optionally purifying the product of Step 5

Any subset of the above steps can be performed in any order any number of times. In some embodiments, the barcode oligonucleotide is combined with the fusion with the barcode oligonucleotide in excess, or vice versa. In some embodiments, the fusion is combined with the primary probe with the primary probe in excess, or vice versa. In some embodiments, the fusion is expressed and optionally purified prior to Step 1.

In some embodiments, the process of assembling the barcode fusion probe comprises some or all of the following steps. In some embodiments, the process of assembling the barcode fusion probe comprises at least step 1. In some embodiments, the process of assembling the barcode fusion probe comprises at least step 2. In some embodiments, the process of assembling the barcode fusion probe comprises at least steps 1-2. In some embodiments, the steps are performed in numerical order.

Step 1: combining a barcode oligonucleotide with a secondary recognition domain comprising a primary-probe-binding domain fused to an enzymatic conjugation domain; whereupon the enzymatic conjugation domain covalently couples to the barcode oligonucleotide, thereby forming a secondary fusion probe.

Step 2: combining the secondary fusion probe with a primary probe comprising a primary recognition domain comprising a target-binding domain; whereupon the primary-probe-binding domain binds to the primary probe, thereby forming the barcode fusion probe.

### Assembly of a Barcode Fusion Probe Comprising a Primary Probe

In some embodiments, the process of assembling a barcode fusion probe comprises some or all of the following steps. In some embodiments, the process of assembling the barcode fusion probe comprises at least step 1. In some embodiments, the process of assembling the barcode fusion probe comprises both steps 1-2. In some embodiments, the steps are performed in numerical order.
Step 1: combining the barcode oligonucleotide with a fusion comprising: a) a primary recognition domain comprising a target-binding domain, and b) an enzymatic conjugation domain, whereupon the enzymatic conjugation domain covalently couples to the barcode oligonucleotide to create a barcode fusion probe
Step 2: optionally purifying the product of Step 1

Any subset of the above steps can be performed in any order any number of times. In some embodiments, the barcode oligonucleotide is combined with the fusion with the barcode oligonucleotide in excess, or vice versa. In some embodiments, the fusion is expressed and optionally purified prior to Step 1.

Multiplex Assays with Barcode Probes and/or Barcode Fusion Probes

In some embodiments, the barcode probes and/or barcode fusion probes for an N-plex assay are assembled separately, where N is a positive integer. In some embodiments, the barcode probes and/or barcode fusion probes for an N-plex assay are assembled separately and then mixed together.

In some embodiments, barcode probe or barcode fusion probe j comprises a primary probe j and a secondary probe j comprising a barcode oligonucleotide j, for j=1, . . . , N. In some embodiments, primary probe j is raised in the same host (optionally of the same isotype) for some or all of j=1, . . . , N. In some embodiments, secondary probe j comprises the same secondary recognition domain comprising the same primary-probe-binding domain for j=1, . . . , N. In some embodiments, secondary probe j remains tightly bound to primary probe j within barcode probe j or barcode fusion probe j for j=1, . . . , N. In some embodiments of barcode probes, secondary probe j comprises the same conjugation site for some or all of j=1, . . . , N. In some embodiments of barcode probes, secondary probe j comprises two or more conjugation sites for some or all of j=1, . . . , N. In some embodiments of barcode probes, secondary probe j comprises two or more of the same conjugation sites for some or all of j=1, . . . , N. In some embodiments of barcode fusion probes, secondary probe j comprises the same enzymatic conjugation domain for some or all of j=1, . . . , N. In some embodiments, the barcode oligonucleotide j is different for each j=1, . . . , N. In some embodiments, the barcode probe j for target j in an N-plex assay comprises: a) a primary probe j raised against target j in the same host (optionally of the same isotype) for some or all of j=1, . . . , N, b) an anti-primary secondary probe j comprising the same conjugation site and a different barcode oligonucleotide for each j=1, . . . , N. In some embodiments, the barcode fusion probe j for target j in an N-plex assay comprises: a) a primary probe j raised against target j in the same host (optionally of the same isotype) for some or all of j=1, . . . , N, b) an anti-primary secondary probe j comprising the same enzymatic conjugation domain and a different barcode oligonucleotide for each j=1, . . . , N.

In some embodiments, primary probe j comprises a primary antibody, antibody fragment, or nanobody raised in the same host (optionally of the same isotype) for j=1, . . . , N. In some embodiments, secondary probe j comprises a secondary recognition domain comprising the same anti-primary antibody, antibody fragment, or nanobody for j=1, . . . , N. In some embodiments, barcode probe j for target j in an N-plex assay comprises: a) a primary probe j comprising a primary antibody, antibody fragment, or nanobody j raised against target j in the same host (optionally of the same isotype) for j=1, . . . , N, b) an anti-host secondary probe j comprising the same antibody, antibody fragment, or nanobody, the same conjugation site, and a different barcode oligonucleotide for each j=1, . . . , N. In some embodiments, barcode fusion probe j for target j in an N-plex assay comprises: a) a primary probe j comprising a primary antibody, antibody fragment, or nanobody j raised against target j in the same host (optionally of the same isotype) for j=1, . . . , N, b) an anti-host secondary probe j comprising the same antibody, antibody fragment, or nanobody, the same enzymatic conjugation domain, and a different barcode oligonucleotide for each j=1, . . . , N.

In some embodiments, barcode probe or barcode fusion probe j comprises primary probe j and a secondary probe j comprising a barcode oligonucleotide j, for j=1, . . . , N. In some embodiments, secondary probe j remains tightly bound to primary probe j within barcode fusion probe j for j=1, . . . , N. In some embodiments, for an N-plex assay: a) zero, one, or more primary probes are raised in host A; b) zero, one, or more primary probes are raised in host B; c) zero, one, or more primary probes are raised in host C, and so on, so that the total list of hosts used to raise primary probes (A, B, C, . . . ) for an N-plex assay involves M hosts where 1≤M≤N. In some embodiments, the primary probes raised in host A bind to secondary probes that comprise the same secondary recognition domain comprising the same primary-probe-binding domain (for example, an anti-A antibody, anti-A antibody fragment, or anti-A nanobody); in some embodiments, the primary probes raised in host B bind to secondary probes that comprise the same secondary recognition domain comprising the same primary-probe-binding domain (for example, an anti-B antibody, anti-B antibody fragment, or anti-B nanobody); in some embodiments, the primary probes raised in host C bind to secondary probes that comprise the same secondary recognition domain comprising the same primary-probe-binding domain (for example, an anti-C antibody, anti-C antibody fragment, or anti-C nanobody); and so on, so that the total of N secondary probes comprises a total of M anti-host recognition domains. In some embodiments of barcode probes, all secondary probes comprising the same secondary recognition domain further comprise the same conjugation site (total of P conjugation sites with 1≤P≤M).

In some embodiments, all N secondary probes comprise the same conjugation site (P=1). In some embodiments of barcode fusion probes, all secondary probes comprising the same secondary recognition domain further comprise the same enzymatic conjugation domain, wherein the secondary recognition domain is fused to the enzymatic conjugation domain (total of L enzymatic conjugation domains with 1≤L≤M). In some embodiments, all N secondary probes comprise the same enzymatic conjugation domain (L=1). In some embodiments, each of L enzymatic conjugation domains recognizes a different conjugation domain with a specific sequence within a barcode oligonucleotide (enzymatic conjugation domain j recognizes conjugation domain j for j=1, . . . , L). In some embodiments of barcode probes or barcode fusion probes, each barcode oligonucleotide comprises a different barcode domain (barcode domain j for target j=1, . . . , N). In some embodiments, barcode domain j mediates generation of a distinguishable signal j, for j=1, . . . , N.

### HCR Imaging

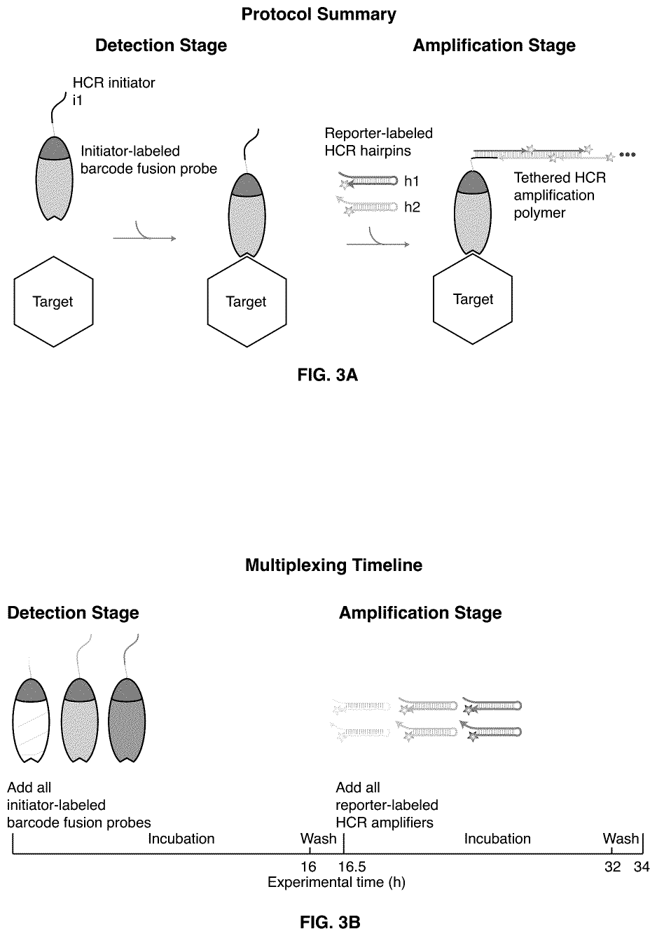

In some embodiments, N targets, wherein N is a positive integer, are imaged in a sample using N barcode probes and/or barcode fusion probes each comprising a different barcode oligonucleotide comprising a different HCR initiator (target j detected by barcode probe or barcode fusion probe j comprising HCR initiator j for j=1, . . . , N), as well as N HCR amplifiers, each comprising a distinguishable reporter (HCR amplifier j comprising distinguishable reporter j for j=1, . . . , N). In some embodiments, an N-plex assay is performed using a 2-stage protocol (for example, FIGS. 3AB, 4AB and 21AB). In some embodiments, during the Detection Stage, all N barcode probes and/or barcode fusion probes are introduced to the sample and bind their cognate targets through binding of the target-binding domain to the target; unused probes are washed from the sample. In some embodiments, during the Amplification Stage, all N HCR amplifiers are introduced to the sample and HCR initiators trigger growth of tethered HCR amplification polymers; unused HCR hairpins are washed from the sample. In some embodiments, the sample is then imaged to detect N distinct signals generated directly or indirectly by the N distinct reporters.

In some embodiments, a barcode fusion probe comprises a secondary probe comprising a secondary recognition domain fused to an enzymatic conjugation domain covalently coupled to a barcode oligonucleotide. In some embodiments, the target for the secondary probe is a primary probe bound within the sample. In some embodiments, N targets are imaged in a sample using N primary probes each raised in a different host organism (target j detected by primary probe j raised in host j for j=1, . . . , N), N secondary barcode fusion probes each comprising a different anti-host secondary recognition domain and a different barcode oligonucleotide comprising a different HCR initiator (primary probe j detected by anti-host-j secondary barcode fusion probe j comprising HCR initiator j for j=1, . . . , N), as well as N HCR amplifiers, each comprising a distinguishable reporter (HCR amplifier j comprising distinguishable reporter j for j=1, . . . , N). In some embodiments, an N-plex assay is performed using a 2-stage protocol (for example, see FIG. 13D). In some embodiments, during the Detection Stage: a) all N primary probes are introduced to the sample and bind their cognate targets; unused probes are washed from the sample; b) all N secondary barcode fusion probes are introduced to the sample and bind their cognate primary probes; unused probes are washed from the sample. In some embodiments, during the Amplification Stage: all N HCR amplifiers are introduced to the sample and HCR initiators trigger growth of tethered HCR amplification polymers; unused HCR hairpins are washed from the sample. In some embodiments, the sample is then imaged to detect N distinct signals generated directly or indirectly by the N distinct reporters.

In some embodiments, N targets, target complexes, and/or sets of proximal targets, where N is a positive integer, are imaged in a sample using N probe units each comprising two or more fractional-initiator barcode probes and/or fractional-initiator barcode fusion probes (target j detected by a probe unit j comprising two or more barcode probes and/or barcode fusion probes that colocalize full HCR initiator j for j=1, . . . , N), as well as N HCR amplifiers, each comprising a distinguishable reporter (HCR amplifier j comprising distinguishable reporter j for j=1, . . . , N). In some embodiments, colocalization of the fractional initiators within probe unit j is mediated by one or more proximity probes j for j=1, . . . , N.

### Signal Generation

In some embodiments, a barcode oligonucleotide is used to mediate generation of a signal directly or indirectly. In some embodiments, a barcode oligonucleotide mediates signal amplification.¹⁶⁻¹⁸ In some embodiments, a barcode oligonucleotide mediates signal amplification via HCR,¹⁹⁻²³ branched DNA (bDNA),²⁴⁻³⁰ rolling circle amplification (RCA),³¹⁻³⁵ catalytic reporter deposition (CARD),^{18,28,26-56} polymerase chain reaction (PCR),^{27,57} proximity ligation assay (PLA),⁵⁸⁻⁶⁹ and/or any other signal amplification method that increases the signal intensity. In some embodiments, the barcode oligonucleotide binds to a readout probe comprising one or more reporters that directly or indirectly lead to generation of a signal.

### Hybridization Chain Reaction (HCR) Signal Amplification

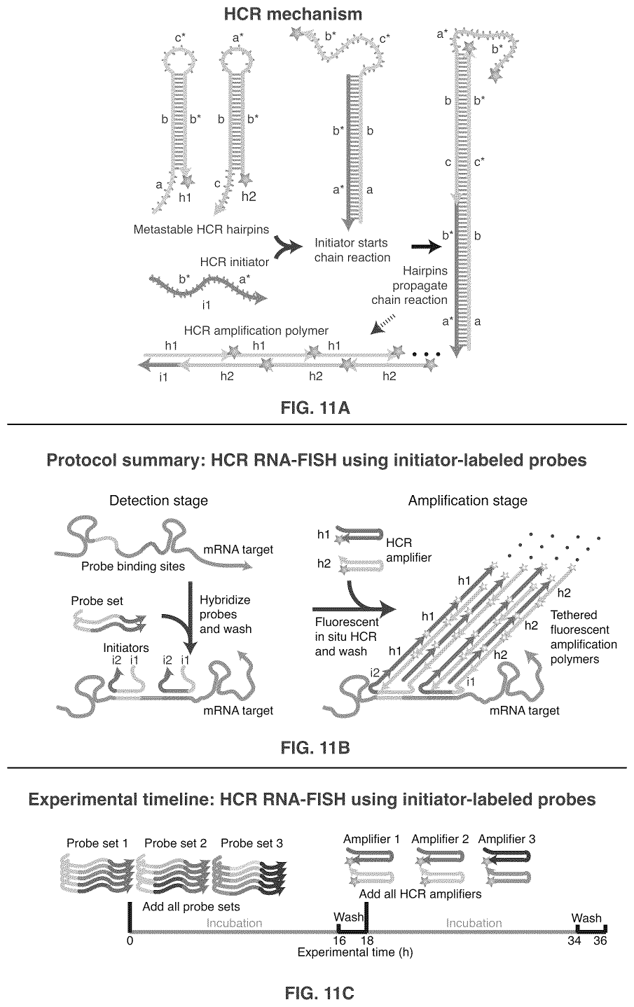

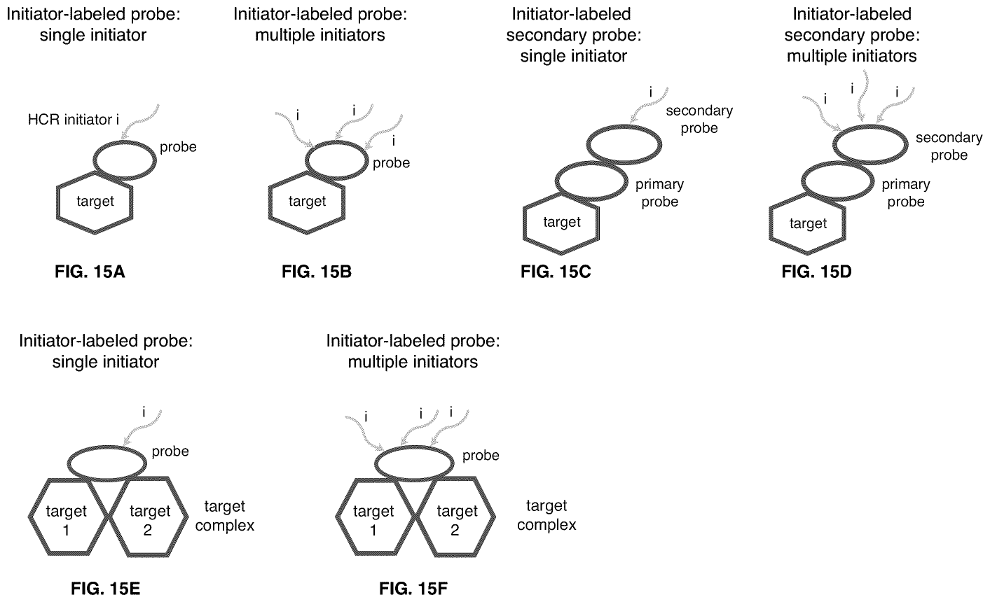

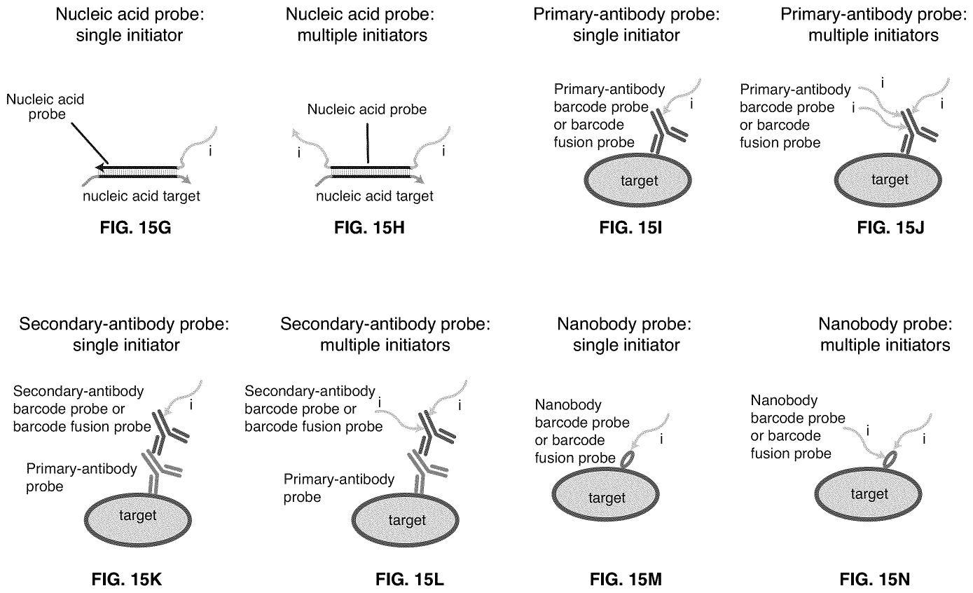

In some embodiments, signal amplification based on the mechanism of hybridization chain reaction (HCR) enables detection of molecular targets within a sample. For example, detection of protein, RNA, DNA, other molecules, and/or complexes thereof in fixed cells, tissues, or whole-mount embryos.^{19-23,70,71} A target can be detected using one or more probes that trigger chain reactions in which reporter-labeled HCR hairpins self-assemble into tethered reporter-decorated HCR amplification polymers, generating amplified signals at the site of the target within a sample (for example, see FIGS. 11B, 12A, 13A, and 13C).¹⁹⁻²³ In some embodiments, an HCR amplifier comprises two or more metastable HCR hairpins that co-exist without interacting in the absence of a cognate HCR initiator, but that upon exposure to the cognate HCR initiator undergo a chain reaction of polymerization steps to form an HCR amplification polymer. In some embodiments, an HCR initiator comprises a nucleic acid sequence capable of triggering an HCR amplification cascade. In some embodiments, an initiator-labeled barcode probe (also known as an initiator-labeled probe) or an initiator-labeled barcode fusion probe (also known as an initiator-labeled probe) comprises a target-binding domain and one or more barcode oligonucleotides each comprising one or more HCR initiators (for example, see FIGS. 11B, 13A, 15). For example, a target protein can be detected using an initiator-labeled primary antibody probe comprising a target-binding domain and one or more barcode oligonucleotides comprising one or more HCR initiators (for example, see FIGS. 13A, 15I, and 15J). Also, for example, a target protein can be detected using a barcode probe or a barcode fusion probe comprising a secondary probe bound to a primary probe wherein the primary probe comprises a target-binding domain and the secondary probe comprises one or more barcode oligonucleotides each comprising one or more HCR initiators (for example, see FIGS. 1B, 2B, 2D, 4A-4B, 19, 20A-20B, and 21A-21B).

In some embodiments, a target is detected using a probe set comprising one or more initiator-labeled probes each comprising a target-binding domain and one or more barcode oligonucleotides each comprising one or more HCR initiators (for example, FIGS. 15A-15N). In some embodiments, a target is detected within a sample using a probe set comprising one or more probe units (for example see the probe sets of FIGS. 12A-12B and 22A-22B), where a probe unit comprises two or more fractional-initiator barcode probes (also known as fractional-initiator probes) or two or more fractional-initiator barcode fusion probes (also known as fractional-initiator probes) (for example see the probe units of FIGS. 16A-16D, 17A-17E, 18A-18R, and 22A-22B), where each fractional-initiator barcode probe or fractional-initiator barcode fusion probe comprises a target-binding domain and a barcode oligonucleotide comprising a fractional initiator. In some embodiments, binding of each probe within a probe unit to proximal cognate binding sites on the target colocalizes the fractional initiators to form a full HCR initiator (for example, see the full HCR initiators of FIGS. 12A, 16A-16D, 17A-17E, 18A-18R, and 22A-22B) capable of triggering HCR signal amplification. In some embodiments, each fractional-initiator probe within a probe unit further comprises a proximity domain (for example, see FIGS. 22A-22B). In some embodiments, binding of each fractional-initiator probe within a probe unit to cognate binding sites on proximal targets enables binding of one or more proximity probes to the proximity domains within the probe unit to colocalize the fractional initiators to form a colocalized full HCR initiator (for example, see FIGS. 22A-22B) capable of triggering HCR signal amplification.

In some embodiments, HCR signal amplification increases the signal strength by a factor of 2, 5, 10, 15, 20, 25, 30, 40, 50, 75, 100, 500, 1000, 2000, 5000, 10,000, 20,000, 50,000, or 100,000-fold, or a value with a range defined by any two of the aforementioned values.

In some embodiments, the target-binding domains within a probe unit bind to overlapping or non-overlapping regions of the target. In some embodiments, the fractional initiators within a probe unit hybridize to overlapping or non-overlapping regions of an HCR hairpin. In some embodiments, individual fractional-initiator probes that bind non-specifically do not colocalize a full HCR initiator, suppressing spurious generation of amplified HCR

### Background

In some embodiments, an HCR amplifier comprises two or more HCR hairpins (for example, see the HCR amplifiers of FIGS. 11A-11B). In some embodiments, each HCR hairpin comprises an input domain with a single-stranded toehold and a stem section, and an output domain with a single-stranded loop and a complement to the stem section (for example, see the HCR hairpins of FIGS. 11-14). In some embodiments, the one or more HCR initiators on an initiator-labeled probe each initiate a chain reaction of polymerization steps in which the initiator hybridizes to the input domain of a first HCR hairpin, opening the first hairpin to expose its output domain, which in turn hybridizes to the input domain of a second HCR hairpin, opening the second hairpin to expose its output domain, and so on and so forth, leading to a chain reaction in which hairpins polymerize to yield an HCR amplification polymer tethered to the target (for example, see the amplification polymers of FIGS. 13A and 13C). In some embodiments, in the absence of a full HCR initiator, HCR hairpins are kinetically trapped and do not polymerize, suppressing background. However, if the fractional-initiator probes within a probe unit bind to their adjacent cognate binding sites on the target to colocalize a full HCR initiator, the full HCR initiator initiates a chain reaction of polymerization steps in which the full initiator hybridizes to the input domain of a first HCR hairpin, opening the first hairpin to expose its output domain, which in turn hybridizes to the input domain of a second HCR hairpin, opening the second hairpin to expose its output domain, and so on and so forth, leading to a chain reaction in which hairpins polymerize to yield an HCR amplification polymer tethered to the target (for example, see the amplification polymers of FIG. 12A).

In some embodiments, an HCR hairpin further comprises zero, one, or more reporters that directly or indirectly lead to generation of an amplified signal. In some embodiments, the zero, one, or more reporters on an HCR hairpin serve to mediate an additional layer of signal amplification via catalytic reporter deposition (CARD). In some embodiments, a reporter on an HCR hairpin comprises a fractional reporter such that an auxiliary-reporter-labeled readout probe does not strongly bind the fractional reporter on an individual hairpin, but such that following HCR polymerization, neighboring hairpins in the HCR amplification polymer colocalize a full reporter such that the colocalized full reporter strongly binds an auxiliary-reporter-labeled readout probe. In some embodiments, a readout probe comprises one or more auxiliary reporters and further comprises a reporter-binding domain that binds a reporter on an HCR amplification polymer or binds a full reporter colocalized within an HCR amplification polymer. In some embodiments, amplified signal is generated by one or more reporters or auxiliary reporters associated with an HCR amplification polymer tethered to the target within the sample. In some embodiments, signal is removed from the sample. In some embodiments, HCR signal is generated, detected, and removed from the sample one or more times.

Additional information about HCR, fractional initiators, and uses thereof can be found in U.S. Pat. No. 10,450,599, filed on Jun. 30, 2017, with the title “Fractional Initiator Hybridization Chain Reaction” hereby expressly incorporated by reference in its entirety. Further information about HCR and uses thereof can be found in US20220282300A1, filed on Feb. 22, 2022, with the title “Analysis of Target Molecules within a Sample via Hybridization Chain Reaction”, and PCT/US2024/017915, filed on Feb. 29, 2024, with the title “Probes for Measuring Molecular Proximity in a Sample”, and PCT/US2024/022859, filed on Apr. 3, 2024, with the title “Ultrasensitive Molecular Detection via Hybridization Chain Reaction”, each hereby expressly incorporated by reference in its entirety.

HCR initiators. In some embodiments, an initiator-labeled probe, such as a barcode probe or barcode fusion probe, comprises one or more HCR initiators capable of initiating an HCR polymerization cascade. In some embodiments, an initiator is fully complementary to the input domain of an HCR hairpin such that it hybridizes to the input domain of the hairpin to open the hairpin and initiate the HCR polymerization cascade. In some embodiments, the initiator is partially complementary to the input domain of an HCR hairpin, but sufficiently complementary such that it hybridizes to the input domain of the hairpin to open the hairpin and initiate the HCR polymerization cascade. In some embodiments, the initiator is shorter or longer than the input domain of an HCR hairpin and/or has incomplete complementarity to the input domain of the hairpin, but is able to hybridize to the input domain of the hairpin to open the hairpin and initiate the HCR polymerization cascade. In some embodiments, an HCR initiator might have 60%, 70%, 80%, 90%, or 100% (or any intermediate value between any of these values) complementarity to the input domain of an HCR hairpin, and hybridize to the input domain of the hairpin to open the hairpin and initiate the HCR polymerization cascade. In some situations, initiator-labeled probes comprising one or more initiators may cause increased background due to non-specific binding of initiators to DNA, RNA, proteins, or other molecules within the sample. In some embodiments, the initiators on an initiator-labeled probe are shielded by base-pairing to reduce non-specific binding of the probe within the sample (to reduce background). In some embodiments, the initiator can be shielded by a hairpin structure. In some embodiments, the initiator can be shielded by one or more auxiliary oligos. In some embodiments, the initiator can be shielded by self-complementarity within the oligo comprising an initiator and/or complementarity to one or more auxiliary strands.

Automatic background suppression with HCR fractional-initiator probes. In some embodiments, fractional-initiator probes such as fractional-initiator barcode probes or fractional-initiator barcode fusion probes automatically suppress background because the HCR initiator is split between two or more probes in a probe unit. In some embodiments, if probes within a probe unit bind specifically to the target at proximal cognate binding sites, the target colocalizes the probes within a probe unit to form a full HCR initiator (also known as a colocalized full HCR initiator or a full initiator or a colocalized full initiator). In some embodiments, individual fractional-initiator probes that bind non-specifically do not trigger HCR since each probe carries only a fraction of an HCR initiator, and HCR signal amplification is triggered only if the full HCR initiator is colocalized.

Automatic background suppression with HCR hairpins. In some embodiments, HCR hairpins automatically suppress background because HCR hairpins are kinetically trapped so they do not polymerize in the absence of an HCR initiator. In some embodiments, if the two or more probes within a fractional-initiator probe unit bind specifically to their proximal cognate binding sites on the target, the resulting colocalized full HCR initiator (also known as full HCR initiator or a colocalized full initiator) triggers growth of a tethered HCR amplification polymer (for example, see FIG. 12A). In some embodiments, individual HCR hairpins that bind non-specifically do not trigger HCR since they are kinetically trapped.

Automatic background suppression with HCR fractional-initiator probes and HCR hairpins. In some embodiments, the combination of HCR fractional-initiator probes for target detection and HCR amplification hairpins for signal amplification provide automatic background suppression throughout the protocol, ensuring that reagents will not generate amplified background even if they bind non-specifically.

Full HCR initiator formed by colocalization of 2 or more fractional-initiator probes. We refer to each set of fractional-initiator probes that generate a full HCR initiator (also known as a colocalized full HCR initiator or a full initiator or a colocalized full initiator) as a probe unit (see for example, FIGS. 12A, 16A-16D, 17A-17E, 18A-18R, and 22A-22B). In some embodiments, a full HCR initiator is generated by a pair of fractional-initiator probes that each carry a fraction of the full HCR initiator such that together they comprise the full HCR initiator (fraction f1 for probe P1 and fraction f2 for probe P2 such that f1+f2=1); in this case, a probe unit is two fractional-initiator probes. In some embodiments, the fractions f1 and f2 are sufficiently small compared to the full HCR initiator (for example, f1=0.5 with f2=0.5; or f1=0.45 with f2=0.55; or f1=0.4 with f2=0.6; or f1=0.3 with f2=0.7) such that HCR signal amplification is suppressed if the full HCR initiator is not colocalized by the target.

In some embodiments, an HCR initiator (i1 or i2) is split between three fractional-initiator probes (fraction f1 for probe P1, fraction f2 for probe P2, fraction f3 for probe 3 such that f1+f2+f3=1); in this case, a probe unit consists of three fractional-initiator probes. In some embodiments, an HCR initiator (i1 or i2) is split between N fractional-initiator probes (fraction f1 for probe P1, fraction f2 for probe P2, . . . , fraction fN for probe PN such that f1+f2+ . . . fN=1 with N=2, 3, 4, or more; in this case, a probe unit consists of N fractional-initiator probes. In some embodiments, for any of these values of N, HCR signal amplification is suppressed if the full HCR initiator is not colocalized by the target.

In some embodiments, a full HCR initiator is generated by colocalization of a pair (or set) of probes that each carry a fraction of an HCR initiator such that the sum of the fraction f1 for probe P1 and the fraction f2 for probe P2 (f1+f2) is sufficiently close to 1 (for example, f1=0.47, f2=0.47, f1+f2=0.94; or f1=0.44, f2=0.42, f1+f2=0.86) such that HCR signal amplification is triggered by the colocalized full initiator that results from binding of the pair of probes to their adjacent cognate binding sites on the target. In some embodiments, the fractional-initiator probes within a probe unit generate a full HCR initiator corresponding to 100% of an HCR initiator. In some embodiments, the fractional-initiator probes within a probe unit generate a sufficient fraction of an HCR initiator to provide efficient HCR signal amplification relative to the rate of signal amplification when no fractional-initiator probes are present or when individual fractional-initiator probes are present but are not colocalized by the target. In some embodiments, the fraction of a full HCR initiator generated by colocalized probes within a probe unit is 99%, 95%, 90%, 80%, or 60%, including any range above any one of the preceding values or defined between any two of the preceding values of a full HCR initiator. In some embodiments, a probe unit comprises 2, 3, 4, 5 or more fractional-initiator probes. In some embodiments, the fractional initiators in the probe unit are sufficient to be functional as an HCR initiator when the probes within the probe unit are colocalized by binding to their adjacent cognate binding sites on the target. In some embodiments, while an HCR initiator may have a sequence of a particular length (e.g., 15 nucleotides), the fractional initiators within a probe unit need not be the exact same length. For example, in some embodiments, their combined length could be 14 or 13 nucleotides, if, when colocalized, they still function as an HCR initiator.

In some embodiments, any two or more fractional initiators can be used, as long as, together, they provide the function of an HCR initiator.

In some embodiments, a full HCR initiator is generated by colocalization of a pair of probes that each carry a fraction of an HCR initiator further comprising one or a few or several sequence modifications such that the sum of the fraction f1 for probe P1 and the fraction f2 for probe P2 (f1+f2) is sufficiently close to 1 (for example, f1=0.45, f2=0.47, f1+f2=0.92) such that HCR signal amplification is triggered by the colocalized full initiator that results from binding of the pair of probes to their adjacent cognate binding sites on the target. In some embodiments, the fractional-initiator probes within a probe unit generate a full HCR initiator that has 100% sequence identity with an HCR initiator. In some embodiments, the fractional-initiator probes within a probe unit generate sufficient sequence identity to an HCR initiator to allow efficient HCR signal amplification relative to the rate of signal amplification when no fractional-initiator probes are present or when individual fractional-initiator probes are present but are not colocalized by the target. In some embodiments, the full HCR initiator generated by colocalized probes within a probe unit has 99%, 95%, 90%, 80%, or 60% sequence identity with an HCR initiator, including any range above any one of the preceding values or defined between any two of the preceding values.

Increasing signal strength using helper probes. In order to maintain selectivity for the target, the probe set size is sometimes constrained to be no more than 1 probe or 1 probe unit, or no more than 2 probes or 2 probe units, or no more than N probes or N probe units, where N is less than the number of probes or probe units, N+M, that would be preferentially used to maximize signal strength. In this scenario where the probe set size is constrained by selectivity considerations, the amount of signal generated can be less relative to detection of the same target using N+M probe units both because the probe set has the potential for generating M fewer full initiators for triggering HCR signal amplification and because the absence of the M additional probe units can reduce the binding yield of the N remaining probe units due to cooperative effects.

In some embodiments, probes carry one or more initiators or fractional initiators. In some embodiments, helper probes do not carry initiators or fractional initiators. In some embodiments, a helper probe does not comprise a proximity domain. In some embodiments, in order to increase signal without decreasing selectivity, a probe set comprising N probe units can be augmented with M helper probes.

HCR amplifiers with 2 hairpins. In some embodiments, an HCR amplifier comprises two hairpins (h1 and h2; for example, see FIGS. 11-13). In some embodiments, each hairpin comprises an input domain with a single-stranded toehold and a stem section, and an output domain with a single-stranded loop and a complement to the stem section. In the absence of an HCR initiator (i1 or i2), hairpins h1 and h2 coexist metastably, that is, they are kinetically trapped and do not polymerize.

Initiation with initiator i1. In some embodiments, an initiator i1 comprises a domain complementary to the toehold of hairpin h1 and a domain complementary to the stem section of h1. In some embodiments, if an h1 hairpin encounters initiator i1, the initiator i1 hybridizes to the input domain of hairpin h1 via toehold-mediated strand displacement, opening hairpin h1 to expose the output domain of hairpin h1 and form complex i1-h1. In some embodiments, the output domain of hairpin h1 comprises a domain complementary to the toehold of hairpin h2 and a domain complementary to the stem section of h2. In some embodiments, if an h2 hairpin encounters an i1-h1 complex, the exposed output domain of h1 hybridizes to the input domain of hairpin h2 via toehold-mediated strand displacement, opening hairpin h2 to expose the output domain of hairpin h2 and form complex i1-h1-h2. In some embodiments, the output domain of hairpin h2 comprises a domain complementary to the toehold of hairpin h1 and a domain complementary to the stem section of h1. In some embodiments, if an h1 hairpin encounters an i1-h1-h2 complex, the exposed output domain of h2 hybridizes to the input domain of hairpin h1 via toehold-mediated strand displacement, opening hairpin h1 to expose the output domain of hairpin h1 and form complex i1-h1-h2-h1. In some embodiments, this polymerization process can repeat with alternating h1 and h2 polymerization steps to generate polymers of the form i1-h1-h2-h1-h2-h1-h2 . . . , which we may denote i1-(h1-h2)_{N} for a polymer that incorporates N alternating copies of hairpins h1 and h2. For example, a polymer might incorporate several h1 and h2 molecules, or dozens of h1 and h2 molecules, or hundreds of h1 and h2 molecules, or thousands of h1 and h2 molecules, or tens of thousands of h1 and h2 molecules, or more. In some embodiments, it is possible for a polymer to end with either h1 or h2, so i1-(h1-h2)_{N}-h1 and i1-(h1-h2)_{N}-h1-h2 are both possible, the latter being equivalent to i1-(h1-h2)_{N+1}.

Initiation with initiator i2. In some embodiments, an initiator i2 comprises a domain complementary to the toehold of hairpin h2 and a domain complementary to the stem section of h2. In some embodiments, if an h2 hairpin encounters initiator i2, the initiator i2 hybridizes to the input domain of hairpin h2 via toehold-mediated strand displacement, opening hairpin h2 to expose the output domain of hairpin h2 and form complex i2-h2. In some embodiments, if an h1 hairpin encounters an i2-h2 complex, the exposed output domain of h2 hybridizes to the input domain of hairpin h1 via toehold-mediated strand displacement, opening hairpin h1 to expose the output domain of hairpin h1 and form complex i2-h2-h1. In some embodiments, if an h2 hairpin encounters an i2-h2-h1 complex, the exposed output domain of h1 hybridizes to the input domain of hairpin h2 via toehold-mediated strand displacement, opening hairpin h2 to expose the output domain of hairpin h2 and form complex i2-h2-h1-h2. In some embodiments, this polymerization process can repeat with alternating h2 and h1 polymerization steps to generate polymers of the form i2-h2-h1-h2-h1-h2-h1 . . . , which can be denoted i2-(h2-h1)_{N} for a polymer that incorporates N alternating copies of h2 and h1. For example, a polymer might incorporate several h1 and h2 molecules, or dozens of h1 and h2 molecules, or hundreds of h1 and h2 molecules, or thousands of h1 and h2 molecules, or tens of thousands of h1 and h2 molecules, or more. In some embodiments, it is possible for a polymer to end with either h1 or h2, so i2-(h2-h1)-h2 and i2-(h2-h1)_{N}-h2-h1 are both possible, the latter being equivalent to i2-(h2-h1)_{N+1}.

### HCR Amplifiers

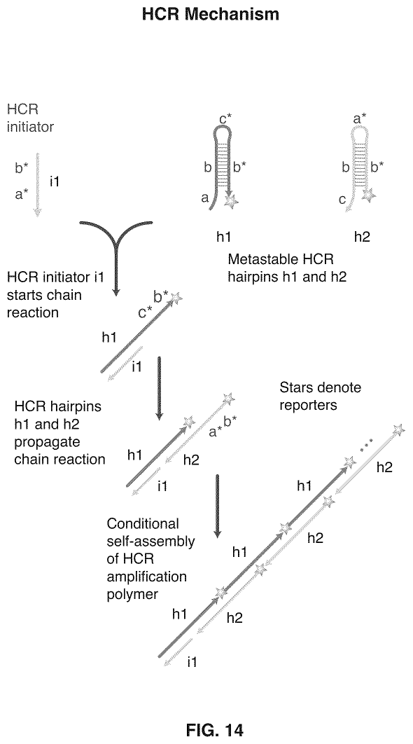

In some embodiments, an HCR amplifier comprises (for example, see FIGS. 11A and 14): 1) a first HCR hairpin (h1) comprising a first input domain (sequence domains “a-b”) comprising a first toehold (sequence domain “a”) and a first stem section (sequence domain “b”), and a first output domain (sequence domains “c*-b*”) comprising a first loop (sequence domain “c*”) and a partner to the first stem section (sequence domain “b*”) that is bound to the first stem section, and 2) a second HCR hairpin (h2) comprising a second input domain (sequence domains “b-c”) comprising a second toehold (sequence domain “c”) and a second stem section (sequence domain “b”), and a second output domain (sequence domains “b*-a*”) comprising a second loop (sequence domain “a*”) and a partner to the second stem section (sequence domain “b*”) that is bound to the second stem section. In some embodiments, a first initiator (i1; sequence domains “b*-a*”) is configured to bind the first input domain. In some embodiments, binding of the first initiator to the first input domain opens the first HCR hairpin to expose the first output domain. In some embodiments, the exposed first output domain is configured to bind the second input domain. In some embodiments, binding of the exposed first output domain to the second input domain opens the second HCR hairpin to expose the second output domain. In some embodiments, the exposed second output domain is configured to bind the first input domain. In some embodiments, binding of the exposed second output domain to the first input domain leads to HCR polymerization in which first and second HCR hairpins are successively opened and added to the growing polymer in alternating fashion.

In some embodiments, a second initiator (i2; domains “c*-b*”) is configured to bind the second input domain. In some embodiments, binding of the second initiator to the second input domain opens the second HCR hairpin to expose the second output domain. In some embodiments, the exposed second output domain is configured to bind the first input domain. In some embodiments, binding of the exposed second output domain to the first input domain opens the first HCR hairpin to expose the first output domain. In some embodiments, the exposed first output domain is configured to bind the second input domain. In some embodiments, binding of the exposed first output domain to the second input domain leads to HCR polymerization in which second and first HCR hairpins are opened and add to the growing polymer in alternating fashion.

In some embodiments, the first and second HCR hairpins are kinetically trapped so that they do not polymerize except in the presence of the first or second HCR initiators.

HCR amplifiers with 4 hairpins. In some embodiments, an HCR amplifier can comprise more than 2 hairpins. For example, an HCR amplifier might comprise 4 hairpins h1, h2, h3, h4. In some embodiments, just as for 2-hairpin HCR, each hairpin comprises an input domain comprising a single-stranded toehold and a stem section, and an output domain comprising a single-stranded loop and a complement to the stem section. In some embodiments, in the absence of an HCR initiator (i1, i2, i3, or i4), hairpins h1, h2, h3, h4 coexist metastably, that is, they are kinetically trapped and do not polymerize. In some embodiments, the output domain of hairpin h1 comprises a domain complementary to the toehold of hairpin h2 and a domain complementary to the stem section of h2; the output domain of hairpin h2 comprises a domain complementary to the toehold of hairpin h3 and a domain complementary to the stem section of h3; the output domain of hairpin h3 comprises a domain complementary to the toehold of hairpin h4 and a domain complementary to the stem section of h4; the output domain of hairpin h4 comprises a domain complementary to the toehold of hairpin h1 and a domain complementary to the stem section of h1. In some embodiments, initiator i1 comprises a domain complementary to the toehold of hairpin h1 and a domain complementary to the stem section of h1; initiator i2 comprises a domain complementary to the toehold of hairpin h2 and a domain complementary to the stem section of h2; initiator i3 comprises a domain complementary to the toehold of hairpin h3 and a domain complementary to the stem section of h3; initiator i4 comprises a domain complementary to the toehold of hairpin h4 and a domain complementary to the stem section of h4. In some embodiments, analogous to the case of 2-hairpin HCR, if a hairpin h1 encounters a initiator i1, the initiator i1 opens hairpin h1 to form complex i1-h1 with an exposed h1 output domain, which in turn opens hairpin h2 to form complex i1-h1-h2 with an exposed h2 output domain, which in turn opens hairpin h3 with an exposed output domain to form complex i1-h1-h2-h3 with an exposed h3 output domain, which in turn opens hairpin h4 to form complex i1-h1-h2-h3-h4 with an exposed h4 output domain, which in turn opens hairpin h1 to form complex i1-h1-h2-h3-h4-h1 with an exposed h1 output domain, and so on and so forth, leading to polymerization via alternating h1, h2, h3, and h4 polymerization steps to generate polymers of the form i1-h1-h2-h3-h4-h1-h2-h3-h4-h1-h2-h3-h4 . . . , which can be denoted i1-(h1-h2-h3-h4)_{N} for a polymer that incorporates N alternating copies of h1, h2, h3, and h4. In some embodiments, it is possible for a polymer to end with h1, h2, h3, or h4, so i1-(h1-h2-h3-h4)_{N}-h1 i1-(h1-h2-h3-h4)_{N}-h1-h2. i1-(h1-h2-h3-h4)_{N}-h1-h2-h3, and i1-(h1-h2-h3-h4)_{N}-h1-h2-h3-h4 are all possible, the latter being equivalent to i1-(h1-h2-h3-h4)_{N+1}. In some embodiments, it is possible for HCR polymerization to be triggered by any of the cognate initiators (i1, i2, i3, or i4). For example, initiation by initiator i3 could generate polymers of the form i3-(h3-h4-h1-h2). In some embodiments, HCR amplifiers with 4 hairpins are convenient for generating a signal that is absent in the unpolymerized state and present in the polymer state.

HCR amplifiers with 2 or more hairpins. More generally, in some embodiments, an HCR amplifier may comprise M HCR hairpins (h1, h2, . . . , hM) with M an integer of 2 or more. In the absence of an HCR initiator (i1, i2, . . . , iM), hairpins h1, h2, . . . , hM coexist metastably, that is, they are kinetically trapped and do not polymerize. In the presence of a cognate HCR initiator, polymerization occurs via alternating polymerization steps analogous to 2-hairpin or 4-hairpin HCR. For example, initiator i1 would lead to growth of polymers of the form i1-(h1-h2 . . . hM) \ for a polymer that incorporates N alternating copies of h1, h2, . . . , hM. It is possible for a polymer to end with any of h1, h2, . . . , hM, soil-(h1-h2 . . . hM)_{N}-h1, i1-(h1-h2 . . . hM)_{N}-h1-h2 . . . , and i1-(h1-h2 . . . hM) \-h1-h2 . . . hM are all possible, the latter being equivalent to i1-(h1-h2 . . . hM)_{N+1}. It is possible for HCR polymerization to be triggered by any of the cognate initiators (i1, i2, . . . , iM). For example, initiator by full initiator i3 could generate polymers of the form i3-(h3 . . . hM-h1-h2).

Reporter-labeled HCR hairpins. For a given HCR amplifier, each HCR hairpin comprises zero, one, or more reporters. Reporters on different hairpins within an amplifier may be the same or different. For example, an amplifier comprising hairpins h1 and h2 might have: 1) the same reporter on h1 and h2, 2) different reporters on h1 and h2, 3) a reporter on h1 but no reporter on h2, 4) a reporter on h2 but no reporter on h1, 5) no reporter on h1 or h2, 6) zero, one, or more reporters on h1 of which zero, one, or more of them are the same or different as zero, one, or more reporters on h2. Similarly, for an HCR amplifier comprising hairpins h1, h2, h3, h4, each hairpin may comprise zero, one, or more reporters (for example 3, 5, or 10 reporters) of which zero, one, or more of them may be the same as zero, one, or more reporters on each of the other hairpins. In some embodiments, one or more of the reporters for a given hairpin can be unique within a mixture of hairpins and/or hairpin reporters. In some embodiments, there are 1, 10, 100, 1000, 10,000, 100,000 or more unique reporters within a mixture (including any range defined between any two of the previous numbers).

In some embodiments, the one or more reporters on a reporter-labeled HCR hairpin directly or indirectly contributes to the generation, alteration, or elimination of a signal. For example, a reporter could be a fluorophore, a chromophore, a luminophore, a phosphor, a FRET pair, a member of a FRET pair, a quencher, a fluorophore/quencher pair, a rare-earth element or compound, a radioactive molecule, a magnetic molecule, an enzyme, an amino acid, a nucleic acid, a peptide, a tag, or any other molecule that facilitates measurement of a signal.

In some embodiments, a reporter decorating a tethered HCR amplification polymer may bind to the reporter-binding domain of a readout probe to directly or indirectly mediate localization of auxiliary reporters in the vicinity of the reporter, which in turn directly or indirectly mediates generation of an amplified signal.

In some embodiments, the reporter can comprise digoxigenin (DIG) that recruits anti-DIG antibody as the readout probe, where the anti-DIG is directly labeled with one or more auxiliary reporters, or with one or more reporters that serve to directly or indirectly mediate localization of auxiliary reporters in the vicinity of the reporter.

In some embodiments, the reporter can comprise a nucleic acid domain that serves as a substrate with full or partial sequence complementarity to a reporter-binding domain within a readout probe that carries one or more auxiliary reporters,

In some embodiments, the reporter can comprise a nucleic acid domain that serves as a substrate with full or partial sequence complementarity to a reporter-binding domain within a readout probe that carries one or more substrates that serve to mediate localization of auxiliary reporters in the vicinity of the reporter.

In some embodiments, the reporter can comprise a nucleic acid domain that serves as a substrate for a readout probe that directly or indirectly mediates localization of auxiliary reporters in the vicinity of the reporter.

In some embodiments, the reporter can comprise a substrate that serves to recruit a readout probe that indirectly mediates localization of auxiliary reporters in the vicinity of the reporter.

In some embodiments, the reporter can comprise a substrate that serves to recruit a readout probe that comprises an enzyme that mediates catalytic reporter deposition (CARD) in the vicinity of the reporter.

In some embodiments, the reporter can comprise biotin that recruits streptavidin (or another biotin-binding molecule) as the readout probe, where the streptavidin is directly labeled with one or more auxiliary reporters, or with one or more substrates that serve to directly or indirectly mediate localization of auxiliary reporters in the vicinity of the reporter.

In some embodiments, the reporter can comprise a hapten that recruits an anti-hapten antibody readout probe or an anti-hapten nanobody readout probe that directly or indirectly mediates localization of reporters in the vicinity of the reporter via CARD signal amplification. For example, the anti-hapten antibody or nanobody readout probe may comprise an enzyme that mediates CARD.

In some embodiments, the reporter can comprise a hapten that recruits an anti-hapten that directly or indirectly mediates localization of auxiliary reporters in the vicinity of the hairpin reporter. For example, the anti-hapten readout probe may comprise an enzyme that mediates CARD.

In some embodiments, the reporter can comprise an enzyme that mediates CARD signal amplification to deposit CARD-reporter molecules in the vicinity of the hairpin.

In some embodiments, the hairpin reporter can comprise zero, one, or more haptens that mediate, directly or indirectly, localization of auxiliary reporters in the vicinity of the haptens.

In some embodiments, a reporter decorating a tethered HCR amplification polymer binds to the reporter-binding domain of a bridging probe to directly or indirectly mediate localization of one or more HCR initiators in the vicinity of the reporter, which in turn triggers growth of a new HCR amplification polymer tethered to the original HCR amplification polymer.

In some embodiments, a reporter can comprise a hapten that recruits an anti-hapten (for example, an antibody, a nanobody, streptavidin, or another molecule) that is labeled with auxiliary reporters.

In some embodiments provided herein, HCR signal amplification is used to mediate catalytic reporter deposition (CARD), leading to even higher signal gain. In some embodiments, the even higher single gain is about 5, 10, 15, 20, 25, 30, 40, 50, 75, 100, 500, 1000, 2000, 5000, 10,000, 20,000, 50,000, or 100,000-fold, or a value with a range defined by any two of the aforementioned values.

Haptens and anti-haptens. In some embodiments, hairpin labels that are substrates comprising a hapten could for example be digoxygenin (DIG), dinitrophenyl (DNP), a fluorophore, biotin, or any small molecule, biological molecule, or non-biological molecule that can recruit an anti-hapten. Examples of anti-haptens include antibodies, nanobodies, streptavidin, aptamers, or any other molecule or complex of molecules that selectively binds a hapten.

Enzymes for HCR-mediated Catalytic Reporter Deposition (CARD). In some embodiments, reporter-decorated HCR amplification polymers mediate signal amplification via catalytic reporter deposition (CARD) by an enzyme that catalyzes a CARD-substrate leading to deposition of CARD-reporters in the vicinity of the HCR amplification polymer.

In some embodiments, the enzyme can be horseradish peroxidase (HRP) (or polymer HRP comprising multiple HRP enzymes) that acts on a CARD-substrate to catalyze deposition a chromogenic CARD-reporter such as AEC, DAB, TMB, or Stay Yellow, or that catalyzes a CARD-substrate to catalyze deposition of a fluorescent CARD-reporter such as fluorophore-labeled tyramide, or that catalyzes deposition of a hapten-labeled CARD-substrate such as biotin-labeled tyramide, where the hapten serves to mediate localization of CARD-reporters in the vicinity of the reporter-decorated HCR amplification polymer.

In some embodiments, the enzyme can be alkaline phosphatase (AP) (or polymer AP comprising multiple AP enzymes) that acts on a CARD-substrate to catalyze deposition of CARD-reporters, for example a chromogenic CARD-reporter such as but not limited to BCIP/NBT, BCIP/TNBT, Napthol AS-MX phosphate+FastBlue BB, Napthol AS-MX phosphate+FastRed TR, StayGreen.

In some embodiments, the enzyme can be glucose oxidase that acts on a CARD-substrate to catalyze deposition of CARD-reporters, for example NBT.

In some embodiments, the enzyme can be any molecule or complex that directly or indirectly mediates localization of CARD-reporters in the vicinity of a reporter-decorated HCR amplification polymer.

In some embodiments, CARD-reporters deposited in the vicinity of tethered HCR amplification polymers are visible to the human eye. In some embodiments, CARD-reporters deposited in the vicinity of tethered HCR amplification polymers are scanned with an instrument to read a signal.

In some embodiments, the enzyme that mediates CARD is deactivated (aka inactivated) after CARD-reporter deposition (for example, using chemical or heat denaturation). For example, the enzyme that mediates CARD can be deactivated using any combination of:
1. Heat (for example, 65° C. or above);
2. Fixative (for example, 4% PFA);
3. Acid (for example, 0.1 M glycine-HCl with 1% Tween 20 at pH 2.2, 0.2N HCl, 10% acetic acid, 10 mM HCl); and
4. Other chemicals (for example, hydrogen peroxide (H₂O₂), hydrogen peroxide+phenol, sodium azide, DEPC, MAB with 10 mM EDTA).

In some embodiments, HRP is inactivated using H₂O₂. In some embodiments, AP is inactivated using a combination of heat and acid. In some embodiments, AP is inactivated with fixative. In some embodiments, deactivation of the enzyme that mediates CARD allows repeated CARD using the same enzyme in combination with different substrates for different targets to allow multiplex target analysis using HCR-mediated CARD. In some embodiments, deactivation of the enzyme that mediates CARD allows repeated CARD using different enzymes in combination with different CARD-substrates for different targets to allow multiplex target analysis using HCR-mediated CARD.

In some embodiments, CARD allows storage of stained samples for 10 or more years to allow reanalysis in compliance with regulatory requirements. In some embodiments, the CARD-stained sample is adequately stable for 1, 2, 3, 4, 5, 6, 7, 8, 9, 10, 11, 12, 13, 14, 15 or more years, and still in compliance with regulatory requirements. In some embodiments, CARD staining provides for long-term storage of archival samples. In some embodiments, CARD provides for storage of formalin fixed paraffin embedded (FFPE) samples for decades. In some embodiments, CARD provides for storage of pathology samples for decades to provide for retrospective scientific and medical studies. In some embodiments, CARD staining provides for long-term storage of archival samples.

Target types. In some embodiments, an initiator-labeled probe comprises a target-binding domain and further comprises one or more HCR initiators. In some embodiments, an initiator-labeled probe can detect a target comprising: any molecule including but not limited to an RNA molecule (for example, mRNA, rRNA, lncRNA, siRNA, shRNA, microRNA, non-coding RNA, synthetic RNA, or modified RNA), a DNA molecule, a non-natural nucleic acid molecule, a protein molecule, a small molecule, a biological molecule, a chemically modified biological molecule, a non-biological molecule, any complex of molecules comprising any combination of RNA, DNA, protein, small molecules, biological molecules, and/or non-biological molecules (for example, an RNA/RNA complex, an RNA/protein complex, a DNA/protein complex, an RNA/DNA/protein complex, a protein/protein complex), a prokaryotic cell, a eukaryotic cell, a virus, or any combination of the above.

In some embodiments, a fractional-initiator probe comprises a target-binding domain and further comprises one or more fractional-initiators. In some embodiments, a probe unit comprises two or more fractional-initiator probes such that the fractional-initiator probes in the probe unit combine to create a full HCR initiator. In some embodiments, a probe unit can detect a target comprising: any molecule including but not limited to an RNA molecule (for example, mRNA, rRNA, lncRNA, siRNA, shRNA, microRNA, non-coding RNA, synthetic RNA, or modified RNA), a DNA molecule, a non-natural nucleic acid molecule, a protein molecule, a small molecule, a biological molecule, a chemically modified biological molecule, a non-biological molecule, any complex of molecules comprising any combination of RNA, DNA, protein, small molecules, biological molecules, and/or non-biological molecules (for example, an RNA/RNA complex, an RNA/protein complex, a DNA/protein complex, an RNA/DNA/protein complex, a protein/protein complex), any collection of proximal molecules or complexes such that the fractional-initiators in the probe unit can colocalize to form a full HCR initiator when the fractional-initiator probes comprising the probe unit are bound to their respective targets within the collection of proximal molecules or complexes, a prokaryotic cell, a eukaryotic cell, a virus, any combination of the above.

In any of the embodiments provided herein, the fractional initiators within a probe unit are designed to be (or are) complementary to non-overlapping regions of an HCR hairpin (for example, regions separated by 0, 1, 2, or more nucleotides), or are designed to be (or are) complementary to overlapping regions of an HCR hairpin (for example, regions that overlap by 1, 2 or more nucleotides), or are designed to be (or are) substantially complementary to an HCR hairpin (for example, complementary except for 0, 1, 2, a few, or several mismatches), or bind an HCR hairpin.

In any of the embodiments provided herein, the target-binding regions within a probe unit bind to non-overlapping regions of the target (for example, regions separated by 0, 1, 2, or more nucleotides or regions separated by 0, 1, 2, or more nanometers), or bind to overlapping regions of the target (for example, regions that overlap by 1, 2 or more nucleotides, or regions that overlap by 1, 2, or more nanometers).

Probe sets for multiplexing. In some embodiments, probe sets are designed for multiplexed assays in which 2, 3, 4, 5, 10, 20, or 100 or more probe sets are used to bind to different targets in the same sample, where 1, 2, 3, 4, 5, 10, 20, or 100 or more of the probe sets comprise one or more initiator-labeled probes, or one or more probe units each comprising two or more fractional-initiator probes. In some embodiments, probe sets are designed for multiplexed assays in which 2, 3, 4, 5, 10, 20, or 100 or more probe sets are used in the same sample, where more than 1%, more than 2%, more than 5%, more than 10%, more than 30%, more than 50%, or 100% of the probe sets comprise one or more initiator-labeled probes, or one or more probe units each comprising two or more fractional-initiator probes.

Probes Configured to Bind to Overlapping or Non-Overlapping Regions of the Target and/or Designed to have Fractional Initiators that Hybridize to Overlapping or Non-Overlapping Regions of an HCR Hairpin.

In some embodiments, a probe unit comprises two or more fractional-initiator probes each comprising a target-binding region and a fractional-initiator.

In some embodiments, the fractional-initiators within a probe unit are complementary to adjacent regions of an HCR hairpin.

In some embodiments, the fractional-initiators within a probe unit are complementary to non-overlapping regions of an HCR hairpin (for example, regions separated by 0, 1, 2, or more nucleotides). In some embodiments, the non-overlapping regions of an HCR hairpin are separated by 0 nucleotides. In some embodiments, the non-overlapping regions of an HCR hairpin are separated by 1 nucleotide. In some embodiments, the non-overlapping regions of an HCR hairpin are separated by 2 or more nucleotides.

In some embodiments, the fractional-initiators within a probe unit are complementary to overlapping regions of an HCR hairpin (for example, regions that overlap by 1, 2 or more nucleotides). In some embodiments, the overlapping regions of an HCR hairpin overlap by 1 nucleotide. In some embodiments, the overlapping regions of an HCR hairpin overlap by 2 or more nucleotides.

In some embodiments, the fractional-initiators within a probe unit are substantially complementary to an HCR hairpin (for example, complementary except for 0, 1, 2, a few, or several mismatches). In some embodiments, the fractional-initiators within a probe unit are substantially complementary to an HCR hairpin with 0 mismatches. In some embodiments, the fractional-initiators within a probe unit are substantially complementary to an HCR hairpin except for 1 mismatch. In some embodiments, the fractional-initiators within a probe unit are substantially complementary to an HCR hairpin except for 2 mismatches. In some embodiments, the fractional-initiators within a probe unit are substantially complementary to an HCR hairpin except for 2 or more mismatches.

In some embodiments, the fractional-initiators within a probe unit are designed to hybridize to adjacent regions of an HCR hairpin. In some embodiments, the fractional-initiators within a probe unit hybridize to adjacent regions of an HCR hairpin.

In some embodiments, the fractional-initiators within a probe unit are designed to hybridize to non-overlapping regions of an HCR hairpin. In some embodiments, the fractional-initiators within a probe unit hybridize to non-overlapping regions of an HCR hairpin.

In some embodiments, the fractional-initiators within a probe unit are designed to hybridize to overlapping regions of an HCR hairpin. In some embodiments, the fractional-initiators within a probe unit hybridize to overlapping regions of an HCR hairpin.

In some embodiments, the fractional-initiators within a probe unit are designed to have sequences that are complementary to adjacent regions of an HCR hairpin. In some embodiments, the fractional-initiators within a probe unit have sequences that are complementary to adjacent regions of an HCR hairpin.

In some embodiments, the fractional-initiators within a probe unit are designed to have sequences that are complementary to non-overlapping regions of an HCR hairpin. In some embodiments, the fractional-initiators within a probe unit have sequences that are complementary to non-overlapping regions of an HCR hairpin.

In some embodiments, the fractional-initiators within a probe unit are designed to have sequences that are complementary to overlapping regions of an HCR hairpin. In some embodiments, the fractional-initiators within a probe unit have sequences that are complementary to overlapping regions of an HCR hairpin.

In some embodiments, the fractional-initiators within a probe unit are designed to have sequences that are substantially complementary to adjacent regions of an HCR hairpin. In some embodiments, the fractional-initiators within a probe unit have sequences that are substantially complementary to adjacent regions of an HCR hairpin.

In some embodiments, the fractional-initiators within a probe unit are designed to have sequences that are substantially complementary to non-overlapping regions of an HCR hairpin. In some embodiments, the fractional-initiators within a probe unit have sequences that are substantially complementary to non-overlapping regions of an HCR hairpin.

In some embodiments, the fractional-initiators within a probe unit are designed to have sequences that are substantially complementary to overlapping regions of an HCR hairpin. In some embodiments, the fractional-initiators within a probe unit have sequences that are substantially complementary to overlapping regions of an HCR hairpin.

In some embodiments, the target-binding regions within a probe unit are able to bind to adjacent regions of the target. In some embodiments, the target-binding regions within a probe unit are able to bind to non-overlapping regions of the target (for example, regions separated by 0, 1, 2, or more nucleotides, or regions separated by 0, 1, 2, or more nm). In some embodiments, the target-binding regions within a probe unit are able to bind to overlapping regions of the target (for example, regions overlapping by 1, 2, or more nucleotides, or regions overlapping by 1, 2, or more nm). In some embodiments, the target-binding regions within a probe unit are able to bind to adjacent regions of the target. In some embodiments, the target-binding regions within a probe unit are able to bind to non-overlapping regions of the target. In some embodiments, the target-binding regions within a probe unit are able to bind to overlapping regions of the target, or have sequences that hybridize to adjacent regions of the target. In some embodiments, the target-binding regions within a probe unit have sequences that hybridize to non-overlapping regions of the target. In some embodiments, the target-binding regions within a probe unit have sequences that hybridize to overlapping regions of the target.

In some embodiments, a probe unit comprises two fractional-initiator probes. In some embodiments, the two fractional-initiator probes bind to the cognate target to colocalize a full HCR initiator. In some embodiments, the colocalized full HCR initiator then binds to the cognate HCR hairpin to initiate HCR polymerization, with one fractional-initiator hybridizing to the hairpin to form a first duplex and the other fractional-initiator hybridizing to the hairpin to form a second duplex. In some embodiments, there is an energetically unfavorable junction between the two duplexes that leads to a kinetic barrier during the branch migration process that opens the first HCR hairpin to initiate polymerization. In some embodiments, by configuring the fractional initiators to bind to overlapping regions of the hairpin, the junction can relax into an energetically more favorable conformation to reduce the height of the kinetic barrier, increasing the efficiency of HCR initiation and/or increasing the affinity between the colocalized full HCR initiator and the first HCR hairpin so as to increase the amount of amplified HCR signal generated in a given period of time. In some embodiments the affinity between the two probes and the cognate target can be increased by configuring the target-binding regions of the two probes to bind to overlapping regions of the target so as to permit the junction between the molecules to relax to an energetically favorable conformation.

In some embodiments, probe sets are designed for multiplexed assays in which 2, 3, 4, 5, 10, 20, or 100 or more probe sets are used to bind to different targets in the same sample, where 1, 2, 3, 4, 5, 10, 20, or 100 or more of the probe sets comprise one or more initiator-labeled probes. In some embodiments, probe sets are designed for multiplexed assays in which 2, 3, 4, 5, 10, 20, or 100 or more probe sets are used in the same sample, where more than 1%, more than 2%, more than 5%, more than 10%, more than 30%, more than 50%, or 100% of the probe sets comprise one or more initiator-labeled probes.

In some embodiments, probe sets are designed for multiplexed assays in which 2, 3, 4, 5, 10, 20, or 100 or more probe sets are used to bind to different targets in the same sample, where 1, 2, 3, 4, 5, 10, 20, or 100 or more of the probe sets comprise one or more probe units comprising fractional initiators that hybridize to overlapping regions of an HCR hairpin. In some embodiments, probe sets are designed for multiplexed assays in which 2, 3, 4, 5, 10, 20, or 100 or more probe sets are used in the same sample, where more than 1%, more than 2%, more than 5%, more than 10%, more than 30%, more than 50%, or 100% of the probe sets comprise one or more probe units comprising fractional initiators with sequences that are complementary to overlapping regions of an HCR hairpin.

In some embodiments, probe sets are designed for multiplexed assays in which 2, 3, 4, 5, 10, 20, or 100 or more probe sets are used to bind to different targets in the same sample, where 1, 2, 3, 4, 5, 10, 20, or 100 or more of the probe sets comprise one or more probe units comprising target-binding regions that bind to overlapping regions of a target. In some embodiments, probe sets are for multiplexed assays in which, 3, 4, 5, 10, 20, or 100 or more probe sets are used in the same sample, where more than 1%, more than 2%, more than 5%, more than 10%, more than 30%, more than 50%, or 100% of the probe sets comprise one or more probe units comprising target-binding regions that bind to overlapping regions of a target.

In some embodiments, probe sets are designed for multiplexed assays in which 2, 3, 4, 5, 10, 20, or 100 or more probe sets are used to bind to different targets in the same sample, where 1 or more of the probe sets comprise one or more initiator-labeled probes and 1 or more of the probe sets comprise one or more probe units each comprising two or more fractional-initiator probes. In some embodiments, probe sets are for multiplexed assays in which 2, 3, 4, 5, 10, 20, or 100 or more probe sets are used in the same sample, where more than 0.1%, more than 1%, more than 2%, more than 5%, more than 10%, more than 30%, or more than 50% of the probe sets comprise one or more initiator-labeled probes, and where more than 0.1%, more than 1%, more than 2%, more than 5%, more than 10%, more than 30%, or more than 50% of the probe sets comprise one or more probe units each comprising two or more fractional-initiator probes.

In some embodiments, a probe unit comprises fractional initiators that bind to overlapping regions of an HCR hairpin, where the overlapping regions overlap by 1 base, or 2 bases, or 3 bases, or 4 bases, or 5 bases, or more bases.

In some embodiments, a probe unit comprises target-binding regions that bind to overlapping regions of the target, where the overlapping regions overlap by at least 0.1 nm, or at least 0.2 nm, or at least 0.3 nm, or at least 0.5 nm, or at least 1 nm, or at least 2 nm, or at least 3 nm, or at least 5 nm. In some embodiments, a probe unit comprises target-binding regions comprising sequences that bind to overlapping regions of the target, where the overlapping regions overlap by at least 1 base, or 2 bases, or 3 bases, or 4 bases, or 5 bases, or more bases.

### Materials and Compositions of Initiator-Labeled Probes.

In some embodiments, an initiator-labeled probe comprises one or more target-binding domains and one or more HCR initiators (for example, see FIGS. 15A-15N). In some embodiments, each domain may comprise one or more materials including DNA, RNA, 2′OMe-RNA, PNA, XNA, chemically modified nucleic acids, synthetic nucleic acid analogs, amino acids, chemical linkers, synthetic amino acid analogs, and/or any other molecule suited for the purpose of the domain.

In some embodiments, an initiator-labeled probe may comprise one or more initiators made of DNA and a target-binding domain made of DNA.

In some embodiments, an initiator-labeled probe may comprise one or more initiators made of DNA, a chemical linker, and a target-binding domain made of amino acids (for example, an antibody or a nanobody or an antibody fragment).

In some embodiments, an initiator-labeled probe may comprise an initiator made of a synthetic nucleic acid analog and a target-binding domain made of a combination of DNA and 2′OMe-RNA.

In some embodiments, an initiator-labeled probe may comprise an initiator made of 2′OMe-RNA and a target-binding domain made of a combination of RNA and protein.

In some embodiments, an initiator-labeled probe may comprise an initiator made of DNA and a target-binding domain made of PNA.

In some embodiments, an initiator-labeled probe may comprise one or more initiators made of any nucleic acid or nucleic acid analog and one or more target-binding domains made of any combination of materials suitable for binding the target molecule.

In some embodiments, an initiator-labeled probe may comprise a single covalently linked molecule or may comprise two or more molecules (each covalently linked) that interact non-covalently to form a complex.

In some embodiments, an initiator-labeled probe may comprise an initiator made of DNA that is covalently linked to a target-binding domain made of DNA.

In some embodiments, an initiator-labeled probe may comprise one or more initiators made of DNA that are covalently linked to dCas9 (or another Cas) which is non-covalently bound to a guide RNA (gRNA) such that the target-binding domain comprises the gRNA: dCas9 complex (or gRNA: Cas complex using another Cas).

In some embodiments, an initiator-labeled probe may comprise one or more initiators made of DNA that are covalently linked to a gRNA that is non-covalently bound to dCas9 (or another Cas) such that the target-binding domain comprises the gRNA: dCas9 complex (or gRNA: Cas complex using another Cas).

In some embodiments, an initiator-labeled probe may comprise an initiator made of a nucleic acid or nucleic acid analog that is covalently linked or non-covalently bound to a target-binding domain comprising one or more molecules.

Materials and composition of fractional-initiator probes. In some embodiments, a fractional-initiator probe comprises one or more target-binding domains and one or more fractional initiators (for example, see FIGS. 16A-16D, 17A-17E, and 18A-18R). In some embodiments, a fractional-initiator probe may further comprise one or more proximity domains (see for example, FIGS. 22A-22B). In some embodiments, each domain may comprise one or more materials including DNA, RNA, 2′OMe-RNA, PNA, XNA, chemically modified nucleic acids, synthetic nucleic acid analogs, chemical linkers, amino acids, synthetic amino acid analogs, and/or any other molecule suited for the purpose of the domain.

In some embodiments, a fractional-initiator probe may comprise one or more fractional-initiators made of DNA and a target-binding domain made of DNA.

In some embodiments, a fractional-initiator probe may comprise one or more fractional-initiators made of DNA, a chemical linker, and a target-binding domain made of amino acids (for example, an antibody or a nanobody or an antibody fragment).

In some embodiments, a fractional-initiator probe may comprise a fractional-initiator made of a synthetic nucleic acid analog and a target-binding domain made of a combination of DNA and 2′OMe-RNA.

In some embodiments, a fractional-initiator probe may comprise a fractional-initiator made of 2′OMe-RNA and a target-binding domain made of a combination of RNA and protein.

In some embodiments, a fractional-initiator probe may comprise a fractional-initiator made of DNA and a target-binding domain made of PNA.

In some embodiments, a fractional-initiator probe may comprise one or more fractional-initiators made of any nucleic acid or nucleic acid analog and one or more target-binding domains made of any combination of materials suitable for binding the target molecule.

In some embodiments, a proximity domain may comprise a nucleic acid or a nucleic acid analog.

In some embodiments, a fractional-initiator probe may comprise a single covalently linked molecule or may comprise two or more molecules (each covalently linked) that interact non-covalently to form a complex.

In some embodiments, a fractional-initiator probe may comprise a fractional-initiator made of DNA that is covalently linked to a target-binding domain made of DNA.

In some embodiments, a fractional-initiator probe may comprise one or more fractional-initiators made of DNA that are covalently linked to dCas9 (or another Cas) which is non-covalently bound to a guide RNA (gRNA) such that the target-binding domain comprises the gRNA: dCas9 complex (or gRNA: Cas complex using another Cas).

In some embodiments, a fractional-initiator probe may comprise one or more fractional-initiators made of DNA that are covalently linked to a gRNA that is non-covalently bound to dCas9 (or another Cas) such that the target-binding domain comprises the gRNA: dCas9 complex (or gRNA: Cas complex using another Cas).

In some embodiments, a fractional-initiator probe may comprise a fractional-initiator made of a nucleic acid or nucleic acid analog that is covalently linked or non-covalently bound to a target-binding domain comprising one or more molecules. Each fractional-initiator probe within a probe unit may have the same or different material compositions from the other fractional-initiator probes in the probe unit. Each fractional-initiator probe within a probe unit may have target-binding regions that bind to different detection sites on the same target molecule, or to different detection sites within a target molecular complex, or to different detection sites within a target collection of proximal molecules or complexes.

Removal of Signal from the Sample.

In some embodiments, HCR signal is removed from the sample after detecting the signal. In some embodiments, signal can be removed from the sample by any method that reduces the number of signal-generating reporters and/or auxiliary reporters and/or tertiary reporters and/or first reporters and/or second reporters and/or third reporters, for example: photobleaching fluorescent reporter molecules using light and/or chemical reagents, chemically cleaving reporters from HCR hairpins and flowing them from the sample (e.g., TCEP), chemically cleaving reporters from readout probes and flowing them from the sample, chemically cleaving hairpins to fragment HCR amplification polymers and flowing the fragments from the sample, chemically cleaving probes to untether HCR amplification polymers from the target and flowing the untethered amplification polymers from the sample, using an auxiliary strand to dehybridize hairpins from HCR amplification polymers and flowing the hairpins from the sample, using an auxiliary strand to dehybridize readout probes from HCR amplification polymers and flowing the readout probes from the sample, using chemical denaturants and/or elevated temperature to destabilize HCR amplification polymers and then flowing the hairpins from the sample, using chemical denaturants and/or elevated temperature to destabilize the interaction between probes and their targets and then flowing the untethered amplification polymers from the sample, using chemical denaturants and/or elevated temperature to destabilize the interaction between readout probes and their substrates and then flowing the readout probes from the sample, using enzymes to degrade amplification polymers and/or probes and flowing the degraded molecules from the sample, using DNases to degrade DNA amplification polymers and/or DNA probes and/or DNA targets and flowing the resulting molecules from the sample, using RNases to degrade RNA targets and then flowing the untethered amplification polymers from the sample, using proteases to degrade protein targets and then flowing the untethered amplification polymers from the sample, using a combination of RNases to degrade RNA targets and DNases to degrade DNA amplification polymers and/or DNA probes and flowing the resulting molecules from the sample, using a combination of proteases to degrade protein targets and DNases to degrade DNA amplification polymers and/or DNA probes and/or DNA targets and flowing the resulting molecules from the sample, using two or more of the above methods, or any other method for removing signal from the sample, at the same time or at different times.

Assay formats. In some embodiments, an HCR signal can be measured in one or more different assay formats including but not limited to: blots, northern blots, western blots, Southern blots, spot blots, paper assays, flow cytometry assays, fluorescent flow cytometry assays, cell sorting assays, fluorescence-activated cell sorting assays, magnetic-activated cell sorting assays, microscopy assays, light microscopy assays, epifluorescence microscopy assays, confocal microscopy assays, light sheet microscopy assays, microarray assays, bead-based assays, mass spectrometry assays, fluorescent microscopy assays, mass spectrometry microscopy assays, mass spectrometry flow cytometry assays, fluorescence assays, chemiluminescence assays, bioluminescence assays, colorimetric assays, electrochemical impedance assays, electrochemical chemiluminescence assays, energy dissipation assays, assays using the human eye, assays using a cell phone camera, gel electrophoresis assays, in situ hybridization (ISH) assays, RNA-ISH assays, DNA-ISH assays, immunohistochemistry (IHC) assays, autoradiography assays, or any assay capable of detecting a signal generated by an HCR amplification polymer.

Sample types. In some embodiments, an HCR initiator-labeled probe and/or HCR fractional-initiator probe can be used with HCR amplification hairpins to detect a target in a sample, the target comprising a molecule, a complex, or a collection of proximal molecules or complexes. The target molecule may be contained within a sample, including for example: a mammal, a mammalian specimen, a mammalian sample, a bacterium, a zebrafish embryo, a chicken embryo, a mouse embryo, a human biopsy specimen, a human tissue section, an FFPE tissue section, a urine sample, a blood sample, a stool sample, a mouse tissue section, a brain slice, a sea urchin embryo, a nematode larva, a fruit fly embryo, a model organism, a non-model organism, a multi-species mixture of organisms, an environmental sample containing unknown organisms, a consortium of organisms (for example, a mixture of protists and bacteria within the gut of another organism), a termite, a microbiome, a clinical specimen, a diagnostic sample, a sputum sample, a tumor biopsy sample, a research sample, a sample comprising material from a human, a sample comprising material from a pet (for example, a dog, cat, rabbit, lizard, snake, or fish), material from a wild animal (for example, a cheetah, elephant, rhinoceros, or chimpanzee), material from an extinct animal (for example, a woolly mammoth, a dodo, a giant auk, a triceratops, or a passenger pigeon), living cells (for example, bacteria or cultured mammalian cells), or a living organism (for example a living mouse or a living human).

In some embodiments, the target may be free in solution within the sample. For example, the target may be free in solution within: a test tube, a cell, an embryo, an organism, a tissue section, a biological specimen, or other sample.

In some embodiments, the target may be covalently crosslinked or non-covalently bound to one or more capture probes covalently or non-covalently attached to a solid support. For example, bound directly or indirectly to a capture probe covalently linked to a microarray or bead.

In some embodiments, the target may be fixed, covalently crosslinked, or non-covalently bound directly or indirectly to a solid support. For example, the target may be bound, fixed, or covalently cross-linked to a slide, a blot, a membrane, a paper substrate, or any other substrate. The target may be fixed or covalently crosslinked to a cell, embryo, organism, tissue section, biological specimen, or any other sample. The target may be covalently linked within a sample that is fixed and permeabilized, fixed but not permeabilized, or not fixed but permeabilized.

In some embodiments, the target may be free within a living cell, living embryo, living organism, living ecosystem, or consortium of organisms (for example, the microbiome within the gut of a mammal). The target may be associated with but exterior to a cell or organism, or it may be contained within a cell or organism. The target may be covalently crosslinked within a living cell, living embryo, living organism, living ecosystem, or living consortium of organisms. The target may be present within or absent from one or more cell types within the sample. The target may be present within or absent from one or more species of organism within the sample. The target may be present in a sample that contains one or more off-targets that have different degrees of similarity to the target molecule. The target may be present within an expanded sample. The target may be present within a compressed sample. The sample may be expanded prior to detecting the target so as to increase the spatial separation between molecules. The sample may be compressed prior to detecting the target so as to decrease the spatial separation between molecules. The target and/or other molecules may be crosslinked to an expanded sample so as to maintain the relative position between molecules in the sample as the sample expands. The target and/or other molecules may be crosslinked to a gel, matrix, or other reagents introduced to the sample so as to expand the sample while maintaining the relative position and/or orientation of molecules in the sample as the sample expands. The sample may be differentially expanded and/or compressed with different expansion and/or compression factors in different tissues and/or organs within the sample.

Fixing the sample. In some embodiments, a target molecules can be crosslinked to the sample so that they are retained during subsequent steps in an assay. For example, target molecules can be crosslinked to the sample using chemical reagents (for example, formaldehyde, paraformaldehyde, EDC (1-ethyl-3-(3-dimethylaminopropyl) carbodiimide)).

Permeabilizing the sample. In some embodiments, the sample can be treated to enhance the accessibility of target molecules to HCR probes and amplifiers. For example, the sample (for example, cells, tissue sections, or whole-mount embryos) can be permeabilized using chemical reagents (for example, methanol, ethanol, detergent) or enzymes (for example, proteinase K). Target accessibility can also be enhanced via sample homogenization, microdissection, electroporation, sectioning, heat treatment (for example, Smith J J, Gunasekera T S, Barardi C R, Veal D, Vescy G (2004) J Appl Microbiol 96 (2): 409-417), and/or microwave treatment (for example, Lan H Y, Mu W, NG Y Y, Nikolic-Paterson D H, & Atkins R C (1996) J Histochem Cytochem 44 (3): 281-287). Another option is to deliver HCR probes and amplifiers across the cell membrane using chemical transfection reagents.

Washes to remove unbound reagents from the sample. In some embodiments, background can be reduced by washing unused imaging reagents from the sample. For example, washes can be used to remove probes, HCR initiator-labeled probes, HCR fractional-initiator probes, HCR amplification hairpins, amplification reagents, proximity probes, label probes, antibodies, and/or other imaging reagents from the sample. Washes can be performed at a temperature using chemical reagents such that imaging reagents that are bound specifically are predominantly not removed (retaining signal) and imaging reagents that are bound non-specifically are predominantly removed (reducing background). For example, wash buffers could include denaturing agents (e.g., formamide, urea), salt buffer (e.g., sodium chloride sodium citrate (SSC), phosphate buffered saline (PBS)), acids (e.g., citric acid), surfactants (e.g., Tween 20, Triton-X, SDS), or blocking agents (e.g., tRNA, salmon sperm DNA, BSA, ficoll, polyvinilypyrolidone, heparin). Wash buffer can be combined with wash temperature (e.g., 25-80° C.) to optimize wash stringency.

Accurate and precise target quantitation. In some embodiments, HCR probes and HCR amplifiers provide quantitative analysis of target molecules in an anatomical context, generating a signal that scales approximately linearly with the number of target molecules per imaging voxel. This quantitative property follows from summation of signal that occurs at three levels during imaging: 1) summation over one or more initiator-labeled probes per target molecule or over one or more probe units (each comprising two or more fractional-initiator probes) per target molecule. 2) Summation over multiple HCR amplification hairpins per amplification polymer tethered to an initiator-labeled probe or to a probe unit of fractional-initiator probes that colocalize a full initiator. 3) Summation over zero, one, or more target molecules in an imaging pixel. Quantitative precision can be further increased while still maintaining subcellular resolution by defining imaging voxels that average the intensities of neighboring pixels. The quantitative nature of HCR signal follows from the binding properties of HCR probes, the polymerization properties of HCR amplification hairpins, and the central limit theorem, which leverage summation and averaging during and after image acquisition to generate signals that scale approximately linearly with target abundance. The same quantitative properties apply to other assay formats, with summation and/or averaging occurring during and/or after data acquisition (for example, by a flow cytometer or a blot scanner).

Multiplexing using initiator-labeled probes. In some embodiments, HCR probe sets comprising initiator-labeled probes and HCR amplifiers comprising HCR hairpins can be used for multiplexed target analysis (for example, target analysis via imaging, blotting, flow cytometry, mass cytometry, gel analysis, or any other analysis mode) in which multiple targets are analyzed in the same sample at the same time. Consider a sample containing some or all of N target types of interest as well as zero, one, or more additional off-target species that are not of interest. Each target can be detected using a probe set comprising one or more initiator-labeled probes (each comprising one or more HCR initiators) that selectively bind the cognate target. In some embodiments, the probe set for each of N target types is labeled with HCR initiators for a different HCR amplifier. For example, Target 1 can be detected with Probe Set 1 labeled with HCR initiators for HCR Amplifier 1, Target 2 can be detected with Probe Set 2 labeled with HCR initiators for HCR Amplifier 2, and so on, with Probe Set N labeled with HCR initiators for HCR Amplifier N.

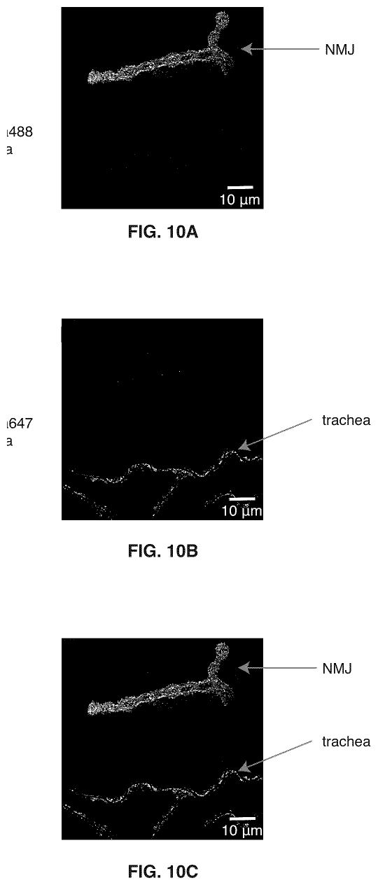

In some embodiments, the N probe sets operate orthogonally such that each probe set selectively binds its cognate target independent of whether the other probe sets and/or targets are present in the sample. In some embodiments, the N amplifiers operate orthogonally such that; 1) the hairpins for each amplifier coexist metastably in the absence of a cognate HCR initiator, 2) each amplifier is selectively triggered to polymerize if its cognate initiator is present independent of whether the other amplifiers are present in the sample. In some embodiments, the reporters carried by each HCR amplifier are orthogonal such that the analysis method is able to measure the signal generated by each HCR amplifier whether or not the other reporters are present in the sample (for example, fluorescent reporters that can be distinguished using fluorescence microscopy, or rare earth reporters that can be distinguished using mass cytometry). For example, multiplexed imaging using initiator-labeled probes and simultaneous HCR signal amplification for all targets are shown in FIG. 10.

In some embodiments, multiplex target analysis for N target types (types j=1, . . . , N) can be achieved using N orthogonal HCR initiator-labeled probe sets to detect all targets simultaneously (with initiator-labeled probes from probe set j binding to target j for target types j=1, . . . , N).

In some embodiments, multiplex target analysis for N target types (types j=1, . . . , N) can be achieved sing N orthogonal HCR amplifiers to amplify the signal for all target types simultaneously (with hairpins from amplifier j polymerizing in response to initiator j to form an amplification polymer j tethered to target type j for j=1, . . . , N).

In some embodiments, multiplex target analysis for N target types (types j=1, . . . , N) can be achieved by analyzing the sample using a measurement device to detect reporter j either directly carried by one or more of the hairpins in amplifier j or indirectly bound to one or more of the hairpins in amplifier j, for target types j=1, . . . , N.

Multiplexing using fractional-initiator probes. In some embodiments, HCR probe sets comprising fractional-initiator probes and HCR amplifiers comprising HCR hairpins can be used for multiplexed target analysis (for example, target analysis via imaging, blotting, flow cytometry, mass cytometry, gel analysis, or any other analysis mode) in which multiple targets are analyzed in the same sample at the same time. Consider a sample containing some or all of N target types of interest as well as zero, one, or more additional off-target species that are not of interest. Each target can be detected using a probe set comprising one or more probe units (each comprising two or more fractional-initiator probes) that selectively bind the cognate target so that each bound probe unit colocalizes a full HCR initiator. In some embodiments, the probe set for each of N target types colocalizes full HCR initiators for a different HCR amplifier. For example, Target 1 can be detected with Probe Set 1 that colocalizes one or more full HCR initiators for HCR Amplifier 1, Target 2 can be detected with Probe Set 2 that colocalizes one or more full HCR initiators for HCR Amplifier 2, and so on, with Probe Set N colocalizing one or more full HCR initiators for HCR Amplifier N.

In some embodiments, the N probe sets operate orthogonally such that each probe set selectively binds its cognate target independent of whether the other probe sets and/or targets are present in the sample. In some embodiments, the N amplifiers operate orthogonally such that; 1) the hairpins for each amplifier coexist metastably in the absence of a cognate full initiator colocalized by the cognate target, 2) each amplifier is selectively triggered to polymerize if its cognate full initiator is colocalized by its cognate target independent of whether the other amplifiers are present in the sample. In some embodiments, the reporters carried by each HCR amplifier are orthogonal such that the analysis method is able to measure the signal generated by each HCR amplifier whether or not the other reporters are present in the sample (for example, fluorescent reporters that can be distinguished using fluorescence microscopy, or rare earth reporters that can be distinguished using mass cytometry).

In some embodiments, multiplex target analysis for N target types (types j=1, . . . , N) can be achieved using N orthogonal HCR fractional-initiator probe sets to detect all targets simultaneously (with fractional-initiator probes from probe set j binding to target j so as to colocalize a full HCR initiator j for each probe unit in probe set j for target types j=1, . . . , N).

In some embodiments, multiplex target analysis for N target types (types j=1, . . . , N) can be achieved using N orthogonal HCR amplifiers to amplify the signal for all target types simultaneously (with hairpins from amplifier j polymerizing in response to full HCR initiator j to form an amplification polymer j tethered to target type j for j=1, . . . , N).

In some embodiments, multiplex target analysis for N target types (types j=1, . . . , N) can be achieved by analyzing the sample using a measurement device to detect reporter j either directly carried by one or more of the hairpins in amplifier j or indirectly bound to one or more of the hairpins in amplifier j, for target types j=1, . . . , N.

Multiplexing using a combination of initiator-labeled probes and fractional-initiator probes. In some embodiments, multiplexed analysis is performed in a sample using initiator-labeled probes to detect one or more targets (of possibly different types) and fractional-initiator probes to detect one or more other targets (of possibly different types). For example, in the same sample, one or more protein targets and one or more small RNA targets could be detected with orthogonal initiator-labeled probes, one or more mRNA targets and/or DNA targets could be detected with orthogonal fractional-initiator probes, and one or more complex targets (comprising a complex of two or more non-covalently linked molecules) could be detected with fractional-initiator probes. In some embodiments, the probe set for each target (comprising one or more initiator-labeled probes or one or more probe units each comprising two or more fractional-initiator probes and optionally one or more proximity probes) would trigger an orthogonal HCR amplifier that generates (directly or indirectly) an orthogonal signal. In some embodiments, HCR signal amplification is performed for all target types simultaneously.

Multiplexing using spectral imaging. In some embodiments, the number of labels that can be distinguished from each other can be increased using spectral analysis. For example, if two fluorophores have overlapping emissions spectra such that measurement of emissions intensity using a bandpass filter would not be able to distinguish between the two labels, they can be distinguished using spectral imaging in which multiple emissions measurements at different wavelengths are used to distinguish between the signal coming from the two labels even though the emissions spectra of the labels are substantially overlapping. Using spectral imaging, in some embodiments, 10 fluorescent dyes can be spectrally distinguished, or 20 fluorescent dyes can be spectrally distinguished, or 30 or more fluorescent dyes can be spectrally distinguished.

Multiplexing using hybrid spectra using multi-reporter polymers. In some embodiments, HCR polymerization proceeds via alternating h1 and h2 polymerization steps so the resulting HCR amplification polymer contains either: 1) the same number of h1 and h2 hairpins, 2) one more h1 hairpin, 3) or one more h2 hairpin. In some embodiments, as the length of the polymer increases, the fraction of h1 hairpins in the polymer approaches 0.5 and the fraction of h2 hairpins in the polymer also approaches 0.5. In some embodiments, hairpin h1 is labeled with reporter r1 and hairpin h2 is labeled with reporter r2. In some embodiments, the signal produced by the HCR amplification polymer is a 1:1 blend of the signal produced by reporter r1 and reporter r2 with a new hybrid spectrum. Consider a set of N reporters with distinct spectra. In some embodiments, the N reporters can be used to create N*(N−1)/2 hybrid spectra corresponding to the number of distinct pairs of reporters that can be selected from the set of N reporters. For example: 1) with 6 reporters it is possible to create 6*5/2=15 hybrid reporter spectra, 2) with 8 reporters it is possible to create 8*7/2=28 hybrid reporter spectra, 3) with 15 reporters it is possible to create 15*14/2=105 hybrid reporter spectra, 4) with 50 reporters it is possible to create 50*49/2=1225 hybrid reporter spectra, 5) with 100 reporters it is possible to create 100*99/2=4950 hybrid reporter spectra.

Multiplexing using repeated reporter detection. In some embodiments, the number of targets that can be analyzed in a sample can be increased using the same N labels to detect multiple targets in successive rounds of analysis. For example, N targets can be imaged using N labels and then removing the signal from the sample and detecting another set of N targets using the same N labels. This approach is applicable for imaging multiple target types in the same sample: 1) regardless of whether the expression levels of the different target types are high, low, variable within each target type, and/or variable across different target types, 2) regardless of whether the expression patterns of the different target types are spatially overlapping or non-overlapping within the sample. In some embodiments, the choice of probe type can be different for different targets and can, optionally, be mixed within any process. In some embodiments: 1) all targets may be detected with initiator-labeled probes, or 2) all targets may be detected with fractional-initiator probes, or 3) one or more targets may be detected with initiator-labeled probes and other targets may be detected with fractional-initiator probes. Thus, the disclosure of one option herein provides the options for the other and for their combination. For example the statement “Providing N probe sets each comprising either: a) one or more HCR initiator-labeled probes, or b) one or more probe units each comprising two or more HCR fractional-initiator probes” implies that any one of the N probe sets can be of either type (a or b), including the possibility that all probe sets are of the same type (all of type a or all of type b) and the possibility that some probe sets are of one type and some probe sets are of the other type (some of type a and some of type b). It is also appreciated that in some embodiments, the types can be mixed. For example, for any of the embodiments where the “either . . . or” is denoted in this context (unless explicitly noted otherwise). In some embodiments, methods for repeated imaging can be combined with CARD, enzyme deactivation, and/or repeated CARD.

Multiplexing using single-molecule barcoding. In some embodiments, the number of targets that can be analyzed in a sample can be increased by analyzing each target molecule in multiple analysis rounds such that the labels used for different target types in different analysis rounds are varied so as to create a distinct barcode for each species of target molecule. The barcode for a given target molecule is then read out as a barcode of signal measurements. For example, using single-molecule imaging to read out the signal of each target molecule as a diffraction-limited dot, consider 3 rounds of imaging. For each given target type, consider assigning a probe set comprising one or more probe units such that each probe unit in a probe set colocalizes a full HCR initiator corresponding to an HCR amplifier comprising HCR hairpins labeled with either red or green reporters depending on the target type and the round of imaging. Then, for example, a target molecule of type 1 can be read out with barcode (red, red, green; denoting a red dot for round 1 using an HCR amplifier labeled with red reporters, a red dot for round 2 using an HCR amplifier labeled with red reporters, and a green dot for round 3 using an HCR amplifier labeled with green reporters), a target molecule of type 2 can have barcode (red, green, red), a target molecule of type 3 can have barcode (red, red, red), a target molecule of type 4 can have barcode (green, red, green), and so on.

In some embodiments, the number of targets that can be analyzed in a sample can be increased by detecting each target molecule in only a fraction of the barcoding rounds. For example, in an assay with 4 rounds, a target molecule of type 1 can be read out with barcode (red, - - - , - - - , red; denoting a red dot for round 1, no dot for round 2, no dot for round 3, a red dot for round 4), a target molecule of type 2 can be read out with barcode (green, red, - - - , - - - ), and so on.

In some embodiments, any one or more of the optional steps of any one or more of the methods herein can be combined with any one or more of the other optional steps of any one or more of the methods herein. In some embodiments, any one or more of the steps that are different for any one or more of the methods herein can be combined with any one or more of the other steps that are different for any one or more of the methods herein. In some embodiments, any one or more of the optional steps that are different for any one or more of the methods herein can be combined with any one or more of the other optional steps that are different for any one or more of the methods herein.

### EXAMPLES

The following examples are non-limiting and other variations within the scope of the skill in the art are also contemplated.

### Example 1—A Fusion Probe Comprising a Nanobody and an Enzymatic Conjugation Domain Efficiently Covalently Couples Itself to a Barcode Oligonucleotide Containing a Coupling Recognition Domain to Yield a Barcode Fusion Probe

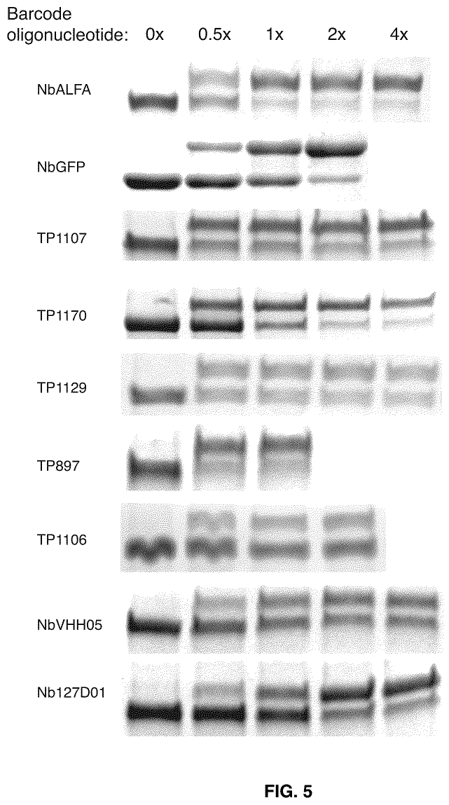

FIG. 5 demonstrates that fusion probes comprising a nanobody fused to a DCV-type HUH enzymatic conjugation domain can efficiently covalently couple to a barcode oligonucleotide comprising a DCV coupling recognition sequence to yield a barcode fusion probe (Table 1). Fusion probe concentrations were calculated against BSA standards. The barcode oligonucleotide and fusion probe were mixed and allowed to react in HUH reaction buffer for 40 minutes at 37° C. with the fusion at 3 μM and the oligonucleotide at stoichiometries of 0×, 0.5×, 1×, 2×, and 4×. The mixture was then analyzed using SDS-PAGE and protein was stained with Coommassie blue dye. Coupling products shift higher in the gel; the location of the unshifted protein-only band is displayed in the Ox lane which does not contain oligonucleotide. The intensity of the shifted band increases and plateaus as oligonucleotide concentration is increased. Results are displayed for covalent coupling of nine different fusion probes, each comprising a different nanobody as indicated on the left.

TABLE 1

| | |
|---|---|
| DCV enzymatic | MAKSGNYSYKRWVFTINNPTFEDYVHVLEFCTLDNCKFAIVG |
| conjugation | EEKGANGTPHLQGFLNLRSNARAAALEESLGGRAWLSRARGS |
| domain | DEDNEEYCAKESTYLRVGEPVSKGRSSDLAEATSAV (SEQ ID |
| | NO: 1) |
| Barcode | 5′-AAAGTATTACCAGAAGAGTATTGAAC-3′ |
| oligonucleotide | (SEQ ID NO: 2) |
| (DCV coupling | |
| recognition | |
| sequence | |
| underlined) | |

### Example 2—Immunofluorescence in Tissue Using Barcode Fusion Probes and HCR Signal Amplification

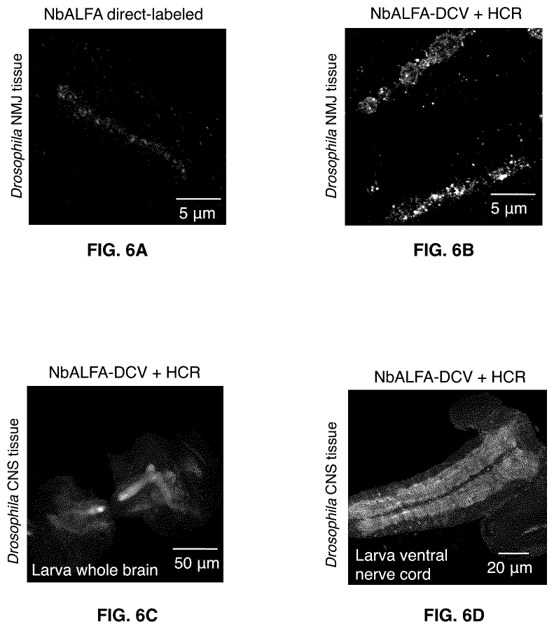

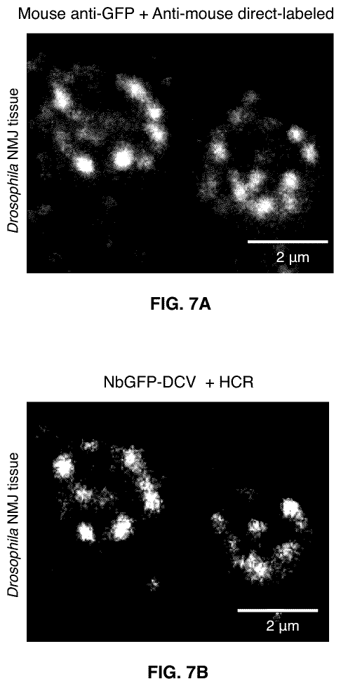

FIGS. 6 and 7 demonstrate immunofluorescence in tissue using barcode fusion probes and HCR signal amplification.

For FIG. 6, the target is ALFA-tagged Dpr10 protein and the barcode fusion probe comprises a nanobody (NbALFA) fused to a DCV enzymatic conjugation domain covalently coupled to a barcode oligonucleotide comprising an HCR initiator. For FIGS. 6A and 6B, Drosophila expressing ALFA-tagged Dpr10 protein were dissected and the neuromuscular junction (NMJ) tissue was stained with either NbALFA direct-labeled with fluorophores (FIG. 6A), or using the barcode fusion probe and performing HCR signal amplification with fluorophore-labeled HCR hairpins (FIG. 6B). Staining intensity increased significantly using HCR signal amplification. FIGS. 6C and 6D demonstrate imaging of ALFA-tagged Dpr10 in the central nervous system (CNS) for the larval whole-brain (FIG. 6C) and the larval ventral nerve cord (FIG. 6D) using the same barcode fusion probe with HCR signal amplification.

For FIG. 7, the target is Bruchpilot protein fused to GFP and the barcode fusion probe comprises a nanobody (NbGFP) fused to a DCV enzymatic conjugation domain covalently coupled to a barcode oligonucleotide comprising an HCR initiator. Drosophila expressing the Bruchpilot-GFP fusion was dissected and the NMJ tissue was immunostained using primary antibody mouse-anti-GFP followed by goat-anti-mouse secondary antibody direct-labeled with fluorophore (FIG. 7A) or imaged using the barcode fusion probe followed by HCR signal amplification with fluorophore-labeled HCR hairpins (FIG. 7B). The same expression pattern is observed in both cases.

### Example 3—Immunofluorescence in Tissue Using Unmodified Primary Antibody Probes, Barcode Fusion Secondary Probes, and HCR Signal Amplification

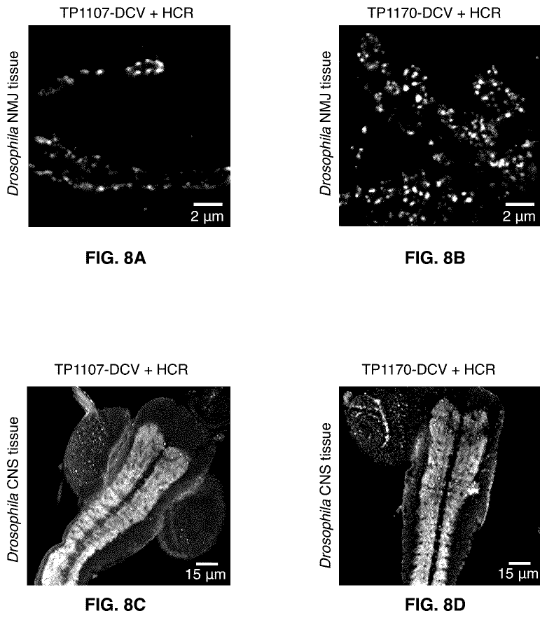

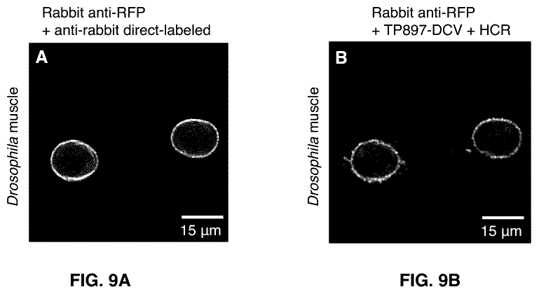

FIGS. 8 and 9 demonstrate immunofluorescence in tissue using unmodified primary antibody probes, barcode fusion secondary probes, and HCR signal amplification.

For FIG. 8, the target is Bruchpilot protein, the primary probe is a mouse anti-Bruchpilot primary antibody, and the secondary probe is a barcode fusion probe comprising either of two nanobodies (TP1107 or TP1170) that bind to different epitopes on the mouse primary antibody fused to a DCV enzymatic conjugation domain covalently coupled to a barcode oligonucleotide comprising an HCR initiator. Wild type Drosophila NMJ (FIGS. 8A and 8B) or CNS (FIGS. 8C and 8D) were dissected and incubated with mouse anti-Bruchpilot, followed by incubation with one of the two barcode fusion probes (TP1107-DCV in FIGS. 8A and 8C; TP1170-DCV in FIGS. 8B and 8D), followed by HCR signal amplification using fluorophore-labeled HCR hairpins. The same staining pattern is observed independent of the nanobody incorporated in the barcode fusion probe.

For FIG. 9, the target is nup-RFP (expresses in the nuclear membrane), the primary probe is rabbit anti-RFP primary antibody, and the secondary probe is a barcode fusion probe comprising a nanobody (TP897) fused to a DCV enzymatic conjugation domain covalently coupled to a barcode oligonucleotide comprising an HCR initiator. Drosophila muscle expressing nup-RFP was either: immunostained using primary antibody rabbit-anti-RFP followed by goat anti-rabbit secondar antibody direct-labeled with fluorophores (FIG. 9A), or imaged using the barcode fusion probe followed by HCR signal amplification with fluorophore-labeled HCR hairpins (FIG. 9B). The same nuclear membrane staining is observed in both cases.

### Example 4—Multiplex Immunofluorescence in Tissue Using Barcode Fusion Probes and HCR Signal Amplification

FIG. 10 demonstrates 2-plex imaging of protein targets using barcode fusion probes each comprising a primary probe bound to an initiator-labeled secondary fusion probe.

For Channel 1 (FIG. 10A), the target is Dlg protein expressed in the neuromuscular junction (NMJ) of Drosophila larvae. The barcode fusion probe comprises a mouse anti-Dlg primary antibody bound to a nanobody (TP1107) fused to a DCV enzymatic conjugation domain covalently coupled to a barcode oligonucleotide comprising HCR initiator B2. HCR signal amplification was performed using HCR amplifier B2-Alexa488 using Alexa488-labeled HCR hairpins.

For Channel 2 (FIG. 10B), the target is Glt-GFP protein which is driven by Glt-Gal4 to express in the trachea of Drosophila larvae. The barcode fusion probe comprises a mouse anti-GFP primary antibody bound to a nanobody (TP1107) fused to a DCV enzymatic conjugation domain covalently coupled to a barcode oligonucleotide comprising HCR initiator B5. HCR signal amplification was performed using HCR amplifier B5-Alexa647 using Alexa647-labeled HCR hairpins.

The barcode fusion probes for both channels incorporate primary probes that are IgG1-type mouse monoclonal antibodies. Furthermore, the secondary probe for each channel incorporates that same nanobody (TP1107). The two barcode fusion probes are used simultaneously in the same sample by first assembling the probes separately (Channel 1: Anti-Dlg mixed with TP1107-DCV-B2; Channel 2, Anti-GFP mixed with TP1107-DCV-B5) and then mixing the probes to perform a multiplexed experiment.

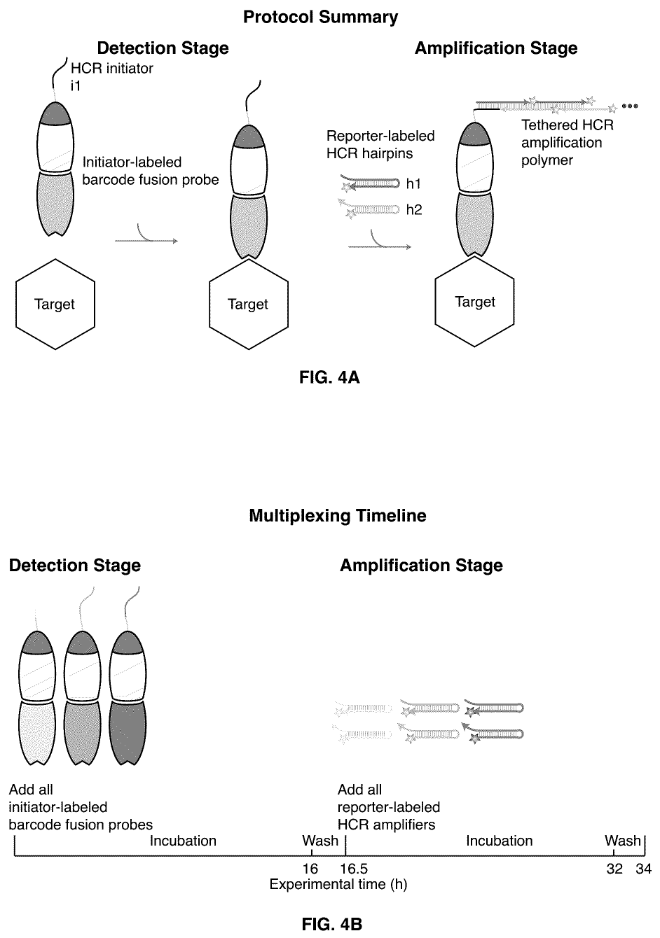

A multiplexed imaging experiment was performed on Glt-GFP larvae using a 2-stage HCR IF protocol (see the protocol summary of FIG. 4B). In the detection stage, a mixture of two initiator-labeled probes was incubated in the sample and then unused probes were washed from the sample. In the amplification stage, a mixture of two fluorophore-labeled HCR amplifiers (each comprising two HCR hairpins) was incubated in the sample and then unused HCR hairpins were washed from the sample.

FIG. 10A displays Channel 1 (Alexa 488), demonstrating that Dlg is detected in the NMJ but not in the trachea (as expected). FIG. 10B displays Channel 2 (Alexa647), demonstrating that GFP is detected in the trachea but not in the NMJ (as expected). FIG. 10C displays a merge of both channels.

These results demonstrate that barcode fusion probes comprising primary antibodies raised in the same host organism can be used simultaneously for multiplex imaging.

### Example 5—Immunofluorescence in Cells Using Unmodified Primary Antibody Probes, Barcode Secondary Probes, and HCR Signal Amplification

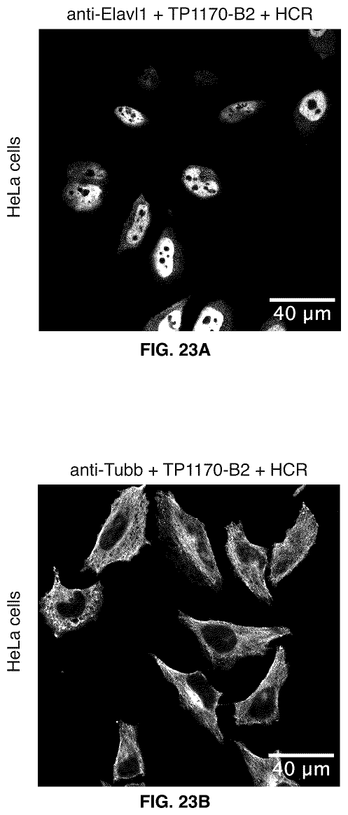

FIGS. 23A-23B demonstrate immunofluorescence in cells using unmodified primary antibody probes, barcode secondary probes, and HCR signal amplification.

For FIG. 23A, the target is ELAV like RNA binding protein 1 (Elav11) predominantly localized to the nucleoplasm. The primary probe is a mouse anti-Elav11 primary antibody, and the secondary probe is a barcode probe comprising nanobody TP1170 (that binds to the mouse primary antibody) covalently coupled to a barcode oligonucleotide comprising an HCR initiator (B2). HeLa cells were fixed and incubated with the primary probe, followed by incubation with secondary barcode probe, followed by HCR signal amplification using fluorophore-labeled HCR hairpins.

For FIG. 23B, the target is tubulin beta protein (Tubb) localized to the microtubules. The primary probe is a mouse anti-Tubb primary antibody, and the secondary probe is a barcode probe comprising nanobody TP1170 (that binds to the mouse primary antibody) covalently coupled to a barcode oligonucleotide comprising an HCR initiator (B2). HeLa cells were fixed and incubated with the primary probe, followed by incubation with the secondary barcode probe, followed by HCR signal amplification using fluorophore-labeled HCR hairpins.

### Example 6—Multiplex Immunofluorescence in Cells Using Barcode Probes and HCR Signal Amplification

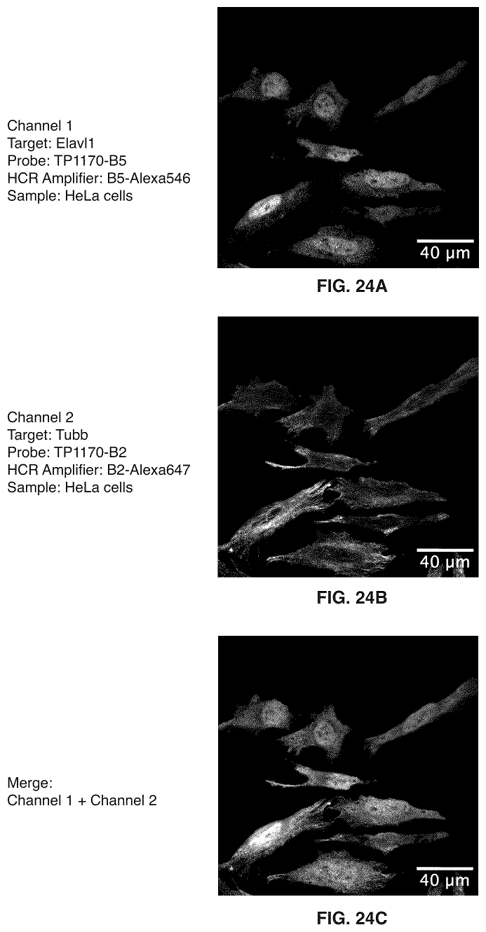

FIGS. 24A-24C demonstrate 2-plex imaging of protein targets using barcode probes each comprising a primary probe bound to an initiator-labeled secondary probe followed by HCR signal amplification.

For Channel 1 (FIG. 24A), the target is ELAV like RNA binding protein 1 (Elav11) predominantly localized to the nucleoplasm of Hela cells. The barcode probe comprises a mouse anti-Elav11 primary antibody bound to a nanobody (TP1170) covalently coupled to a barcode oligonucleotide comprising an HCR initiator (B5). HCR signal amplification was performed using HCR amplifier B5-Alexa546 using Alexa546-labeled hairpins.

For Channel 2 (FIG. 24B), the target is tubulin beta protein (Tubb) localized to the microtubules of Hela cells. The barcode probe comprises a mouse anti-Tubb primary antibody bound to a nanobody (TP1170) covalently coupled to a barcode oligonucleotide comprising an HCR initiator (B2). HCR signal amplification was performed using HCR amplifier B2-Alexa647 using Alexa647-labeled hairpins.

The barcode probes for both channels incorporate primary probes that are IgG1-type mouse monoclonal antibodies. Furthermore, the secondary probe for each channel incorporates the same nanobody (TP1170) covalently conjugated to different barcode oligonucleotide corresponding to a different HCR initiator (initiator for HCR amplifier B5 for Channel 1 and initiator for HCR amplifier B2 for Channel 2). The two barcode probes are used simultaneously in the same sample by first assembling the probes separately (Channel 1: Anti-Elav11 mixed with TP1170-B5; Channel 2: Anti-Tubb mixed with TP1170-B2) and then mixing the probes to perform a multiplex experiment.

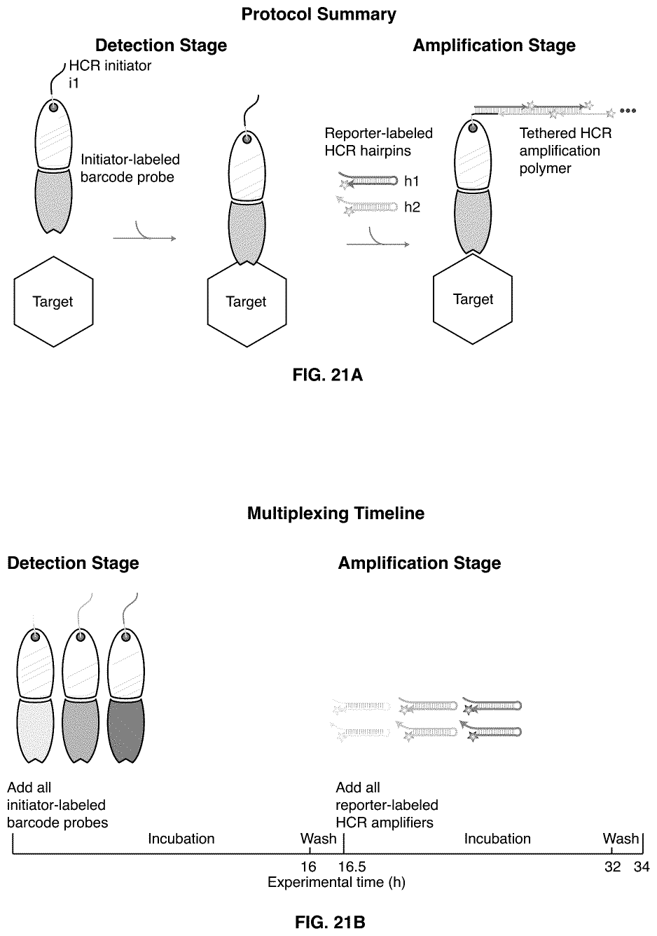

A 2-plex imaging experiment was performed on HeLA cells using a 2-stage HCR IF protocol (see the protocol summary of FIG. 21B). In the detection stage, a mixture of two initiator-labeled barcode probes was incubated in the sample and then unused probes were washed from the sample. In the amplification stage, a mixture of two fluorophore-labeled HCR amplifiers (each comprising two HCR hairpins) was incubated in the sample and then unused HCR hairpins were washed from the sample.

FIG. 24A displays Channel 1 (Alexa 546), demonstrating that Elav11 is predominantly localized in the nucleoplasm and is not localized to the microtubules (as expected). FIG. 24B displays Channel 2 (Alexa647), demonstrating that Tubb is localized to the microtubules and not localized to the nucleoplasm (as expected). FIG. 24C displays a merge of both channels.

These results demonstrate that barcode fusion probes comprising primary antibodies raised in the same host organism can be used simultaneously for multiplex imaging.

### Additional Arrangements

Arrangement 1: A composition comprising a barcode probe comprising: a) a primary probe comprising a primary recognition domain comprising a primary antibody, antibody fragment, nanobody, or polypeptide, wherein the primary antibody, antibody fragment, nanobody, or polypeptide comprises a target-binding domain, b) a secondary probe comprising: i. a secondary recognition domain comprising a secondary antibody, antibody fragment, nanobody, or polypeptide, wherein the secondary antibody, antibody fragment, nanobody, or polypeptide comprises a primary-probe-binding domain and a conjugation site, and ii. a barcode oligonucleotide, wherein the conjugation site is covalently coupled to the barcode oligonucleotide, and wherein the secondary probe is bound to the primary probe.

Arrangement 2: The composition of Arrangement 1, wherein the barcode oligonucleotide comprises an HCR initiator or an HCR fractional initiator.

Arrangement 3: A method of constructing a barcode probe comprising performing the following steps in any order: a) combining a barcode oligonucleotide with a secondary recognition domain comprising a primary-probe-binding domain and a conjugation site; and covalently coupling the conjugation site to the barcode oligonucleotide, thereby forming a secondary probe b) combining the secondary probe with a primary probe comprising a primary recognition domain comprising a target-binding domain; whereupon the primary-probe-binding domain binds to the primary probe, thereby forming the barcode probe.

Arrangement 4: The method of Arrangement 3, further comprising crosslinking the primary probe to the secondary probe.

Arrangement 5: The method of any of the prior arrangements, further comprising purifying the barcode probe.

Arrangement 6: The method of any of the prior arrangements, wherein the barcode oligonucleotide comprises an HCR initiator or an HCR fractional initiator.

Arrangement 7: The method of any of the prior arrangements, wherein the conjugation site is a lysine residue or another natural amino acid.

Arrangement 8: The method of any of the prior arrangements, wherein the conjugation site is a non-natural amino acid.

Arrangement 9: The method of any of the prior arrangements, wherein the secondary probe comprises two or more conjugation sites each covalently coupled to a barcode oligonucleotide.

Arrangement 10: The method of any of the prior arrangements, wherein the secondary recognition domain is expressed prior to covalent coupling of the conjugation site to the barcode oligonucleotide.

Arrangement 11: The method of any of the prior arrangements, wherein the secondary recognition domain is purified prior to covalent coupling of the conjugation site to the barcode oligonucleotide.

Arrangement 12: The method of any of the prior arrangements, wherein the secondary probe is purified prior to binding the secondary probe to the primary probe to form the barcode probe.

Arrangement 13: A composition comprising a panel of N barcode probes for detection of a panel of N targets, wherein N is a positive integer, wherein each barcode probe j (for j=1, . . . , N) comprises: a) a primary probe j comprising a primary recognition domain j comprising a primary antibody, an antibody fragment, a nanobody, or a polypeptide, wherein the primary antibody, antibody fragment, nanobody, or polypeptide comprises a target-binding domain j, a. a secondary probe j comprising: i. a secondary recognition domain j comprising a secondary antibody, an antibody fragment, a nanobody, or polypeptide comprising a primary-probe-binding domain j and a conjugation site j, and ii. a barcode oligonucleotide j, wherein the conjugation site j is covalently coupled to the barcode oligonucleotide j, and wherein the secondary probe j is bound to the primary probe j.

Arrangement 14: The probe panel of Arrangement 13, wherein barcode oligonucleotide j comprises an HCR initiator j or an HCR fractional initiator j for j=1, . . . , N.

Arrangement 15: The probe panel of any one of the prior aspects of Arrangements 13 or 14, wherein the same conjugation site is used for two or more probes in the panel.

Arrangement 16: The probe panel of any one of the prior aspects of Arrangements 13-15, wherein the same secondary recognition domain is used for two or more probes in the panel.

Arrangement 17: The probe panel of any one of the prior aspects of Arrangements 13-16, wherein target-binding domain j is specific to target j and barcode oligonucleotide j is unique to target j for j=1, . . . , N.

Arrangement 18: A method for amplified detection of a target in a sample, the method comprising: a) providing a sample containing a target; b) providing a probe set comprising either: i) an HCR initiator-labeled probe, or ii) a probe unit comprising two or more HCR fractional-initiator probes; c) providing a reporter-labeled first HCR amplifier; d) generating a signal, directly or indirectly, from one or more reporter-decorated HCR amplification polymers; c) detecting the signal; wherein an HCR initiator-labeled probe comprises: a barcode probe comprising: i) a primary probe comprising a target-binding domain and ii) a secondary probe comprising a primary-probe-binding domain and a conjugation site; wherein the secondary probe is bound to the primary probe and the conjugation site is covalently coupled to a barcode oligonucleotide comprising an HCR initiator, wherein an HCR fractional-initiator probe comprises: a barcode probe comprising: i) a primary probe comprising a target-binding domain and ii) a secondary probe comprising a primary-probe-binding domain and a conjugation site; wherein the secondary probe is bound to the primary probe and the conjugation site is covalently coupled to a barcode oligonucleotide comprising an HCR fractional initiator; wherein an HCR amplifier comprises two or more HCR hairpins; wherein an HCR hairpin comprises: an input domain comprising: a single-stranded toehold and a stem section, wherein an HCR hairpin further comprises an output domain comprising: a single-stranded loop and a complement to the stem section, and wherein at least one HCR hairpin further comprises one or more reporters; and wherein when the HCR initiator-labeled probe or probe unit is bound to the target the HCR initiator or colocalized full initiator comprising two or more HCR fractional-initiators initiates HCR signal amplification whereupon the HCR hairpins self-assemble into a tethered reporter-decorated HCR amplification polymer thereby generating the signal.

Arrangement 19: The method of Arrangement 18, wherein a wash step is performed between steps b) and c).

Arrangement 20: The method of any one of the prior aspects of Arrangements 18 or 19, wherein a wash step is performed between steps c) and d).

Arrangement 21: The method of any one of the prior aspects of Arrangements 18-20, wherein the signal is removed from the sample following step e).

Arrangement 22: The method of any one of the prior aspects of Arrangements 18-21, wherein the reporters on the reporter-decorated amplification polymers directly or indirectly mediate CARD signal amplification.

Arrangement 23: The method of any one of the prior aspects of Arrangements 18-22, wherein the target comprises a protein, a peptide, an amino acid, a non-natural amino acid analog, a nucleic acid, a non-natural nucleic acid analog, a tag, a hapten, a fluorophore, a reporter, a chemical, a biological molecule, a pathogen, a small molecule, a macromolecule, a complex of molecules, or a set of molecules in proximity.

Arrangement 24: The method of any one of the prior aspects of Arrangements 18-23, wherein the probe unit comprising two or more HCR fractional-initiator probes further comprises one or more proximity probes.

Arrangement 25: The method of any one of the prior aspects of Arrangements 18-24, wherein each HCR fractional-initiator probe within a probe unit further comprises a proximity domain.

Arrangement 26: The method of any one of the prior aspects of Arrangements 18-25, wherein the one or more proximity probes bind to the proximity domains within a probe unit to colocalize a full HCR initiator capable of triggering HCR signal amplification.

Arrangement 27: The method of any one of the prior aspects of Arrangements 18-26, further comprising repeating any of the steps of the method to detect another target in the sample.

Arrangement 28: A composition comprising a barcode fusion probe comprising a primary probe comprising: a. a primary recognition domain comprising a primary antibody, an antibody fragment, a nanobody, or a polypeptide, wherein the primary antibody, antibody fragment, nanobody, or polypeptide comprises a target-binding domain; b. an enzymatic conjugation domain; and c. a barcode oligonucleotide, wherein the primary recognition domain is fused to the enzymatic conjugation domain, and wherein the enzymatic conjugation domain is covalently coupled to the barcode oligonucleotide.

Arrangement 29: A composition comprising a barcode fusion probe comprising: a. a primary probe comprising a primary recognition domain comprising a primary antibody, antibody fragment, nanobody, or polypeptide, wherein the primary antibody, antibody fragment, nanobody or polypeptide comprises a target-binding domain, b. a secondary probe comprising: i. a secondary recognition domain comprising a secondary antibody, antibody fragment, nanobody, or polypeptide wherein the secondary antibody, antibody fragment, nanobody, or polypeptide comprises a primary-probe-binding domain, ii. an enzymatic conjugation domain, and iii. a barcode oligonucleotide, wherein the secondary recognition domain is fused to the enzymatic conjugation domain, wherein the enzymatic conjugation domain is covalently coupled to the barcode oligonucleotide, and wherein the secondary probe is bound to the primary probe.

Arrangement 30: The barcode fusion probe of any one of the prior aspects of Arrangements 28 or 29, wherein the barcode oligonucleotide comprises an HCR initiator or an HCR fractional initiator.

Arrangement 31: The barcode fusion probe of any one of the prior aspects of Arrangements 28-30, wherein the enzymatic conjugation domain comprises an HUH domain.

Arrangement 32: The barcode fusion probe of Arrangement 31, wherein the HUH domain is DCV.

Arrangement 33: A method of constructing a barcode fusion probe comprising: combining a barcode oligonucleotide with a fusion probe comprising: a) a primary recognition domain comprising a target-binding domain, and b) an enzymatic conjugation domain, whereupon the enzymatic conjugation domain covalently couples to the barcode oligonucleotide.

Arrangement 34: The method of Arrangement 33, further comprising purifying the barcode fusion probe.

Arrangement 35: A method of constructing a barcode fusion probe comprising performing the following steps in any order: a. combining a barcode oligonucleotide with a secondary recognition domain comprising a primary-probe-binding domain fused to an enzymatic conjugation domain; whereupon the enzymatic conjugation domain covalently couples to the barcode oligonucleotide, thereby forming a secondary fusion probe; b. combining the secondary fusion probe with a primary probe comprising a primary recognition domain comprising a target-binding domain; whereupon the primary-probe-binding domain binds to the primary probe, thereby forming the barcode fusion probe.

Arrangement 36: The method of Arrangement 35, further comprising crosslinking the primary probe to the secondary fusion probe.

Arrangement 37: The method of any one of the prior aspects of Arrangements 35 or 36, further comprising purifying the barcode fusion probe.

Arrangement 38: The method of any one of the prior aspects of Arrangements 35-37, wherein the barcode oligonucleotide comprises an HCR initiator or an HCR fractional initiator.

Arrangement 39: The method of any one of the prior aspects of Arrangements 35-38, wherein the enzymatic conjugation domain comprises an HUH domain.

Arrangement 40: The method of Arrangement 39, wherein the HUH domain is DCV.

Arrangement 41: A composition comprising a panel of N barcode fusion probes for detection of a panel of N targets, wherein N is a positive integer, wherein barcode fusion probe j (for j=1, . . . , N) comprises a primary fusion probe j comprising: a. a primary recognition domain j comprising a primary antibody, an antibody fragment, a nanobody, or a polypeptide, wherein the primary antibody, antibody fragment, nanobody or polypeptide comprises a target-binding domain j; b. an enzymatic conjugation domain j; and c. a barcode oligonucleotide j, wherein the primary recognition domain j is fused to the enzymatic conjugation domain j, and wherein the enzymatic conjugation domain j is covalently coupled to the barcode oligonucleotide j.

Arrangement 42: A composition comprising a panel of N barcode fusion probes for detection of a panel of N targets, wherein N is a positive integer, wherein barcode fusion probe j (for j=1, . . . , N) comprises: a. a primary probe j comprising a primary recognition domain j comprising a primary antibody, an antibody fragment, a nanobody, or a polypeptide, wherein the primary antibody, antibody fragment, nanobody, or polypeptide comprises a target-binding domain j; b. a secondary fusion probe j comprising: i. a secondary recognition domain j comprising a secondary antibody, an antibody fragment, a nanobody, or a polypeptide comprising a primary-probe-binding domain j, ii. an enzymatic conjugation domain j, and iii. a barcode oligonucleotide j, wherein the secondary recognition domain j is fused to the enzymatic conjugation domain j, wherein the enzymatic conjugation domain j is covalently coupled to the barcode oligonucleotide j, and wherein the secondary fusion probe j is bound to the primary probe j.

Arrangement 43: The probe panel of any one of the prior aspects of Arrangements 41 or 42, wherein barcode oligonucleotide j comprises an HCR initiator j or an HCR fractional initiator j for j=1, . . . , N.

Arrangement 44: The probe panel of any one of the prior aspects of Arrangements 41-43, wherein the same enzymatic conjugation domain is used for two or more barcode fusion probes in the panel.

Arrangement 45: The probe panel of any one of the prior aspects of Arrangements 41-44, wherein the same secondary recognition domain is used for two or more barcode fusion probes in the panel.

Arrangement 46: The probe panel of any one of the prior aspects of Arrangements 41-45, wherein target-binding domain j is specific to target j and barcode oligonucleotide j is unique to target j for j=1, . . . , N.

Arrangement 47: A method for amplified detection of a target in a sample, the method comprising: a) providing a sample containing a target; b) providing a probe set comprising either: i) an HCR initiator-labeled fusion probe, or ii) a probe unit comprising two or more HCR fractional-initiator fusion probes; c) providing a reporter-labeled first HCR amplifier; d) generating a signal, directly or indirectly, from one or more reporter-decorated HCR amplification polymers; e) detecting the signal; wherein an HCR initiator-labeled fusion probe comprises: a barcode fusion probe comprising a primary probe comprising a target-binding domain wherein either the primary probe or a secondary probe bound to the primary probe comprises a fusion to an enzymatic conjugation domain covalently coupled to a barcode oligonucleotide comprising an HCR initiator, wherein an HCR fractional-initiator fusion probe comprises: a barcode fusion probe comprising a primary probe comprising a target-binding domain wherein either the primary probe or a secondary probe bound to the primary probe comprises a fusion to an enzymatic conjugation domain covalently coupled to a barcode oligonucleotide comprising an HCR fractional initiator; wherein an HCR amplifier comprises two or more HCR hairpins; wherein an HCR hairpin comprises: an input domain comprising: a single-stranded toehold and a stem section, wherein an HCR hairpin further comprises an output domain comprising: a single-stranded loop and a complement to the stem section, and wherein at least one HCR hairpin further comprises one or more reporters; wherein when the HCR initiator-labeled probe or probe unit is bound to the target the HCR initiator or colocalized full initiator comprising two or more HCR fractional-initiators initiates HCR signal amplification whereupon the HCR hairpins self-assemble into a tethered reporter-decorated HCR amplification polymer thereby generating the signal.

Arrangement 48: The method of Arrangement 47, wherein a wash step is performed between steps b) and c).

Arrangement 49: The method of any one of the prior aspects of Arrangements 47 or 48, wherein a wash step is performed between steps c) and d).

Arrangement 50: The method of any one of the prior aspects of Arrangements 47-49, wherein the signal is removed from the sample following step e).

Arrangement 51: The method of any one of the prior aspects of Arrangements 47-50, wherein the target comprises a protein, a peptide, an amino acid, a non-natural amino acid analog, a nucleic acid, a non-natural nucleic acid analog, a tag, a hapten, a fluorophore, a reporter, a chemical, a biological molecule, a pathogen, a small molecule, a macromolecule, a complex of molecules, or a set of molecules in proximity.

Arrangement 52: The method of any one of the prior aspects of Arrangements 47-51, wherein the probe unit comprising two or more HCR fractional-initiator probes further comprises one or more proximity probes.

Arrangement 53: The method of any one of the prior aspects of Arrangements 47-52, wherein each HCR fractional-initiator probe within a probe unit further comprises a proximity domain.

Arrangement 54: The method of any one of the prior aspects of Arrangements 47-53, wherein the one or more proximity probes bind to the proximity domains within a probe unit to colocalize a full HCR initiator capable of triggering HCR signal amplification.

Arrangement 55: The method of any one of the prior aspects of Arrangements 47-54, further comprising repeating any of the steps of the method to detect another target in the sample.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims.

Any of the embodiments, compositions, and/or methods provided herein can be employed with, or in the alternative form of, any of the following. Thus, for example, the above noted compositions and/or methods can employ any of the compositions or methods noted below. Similarly, the above noted compositions and/or methods should be understood to also provide methods employing the methods below or as being part of the methods noted below.

Similarly, the embodiments and/or methods provided herein should also be understood to provide embodiments involved in the method, e.g., compositions, components of the method, kits, etc. In some embodiments, any of the ingredients in one or more of the methods and/or steps provided herein can be provided as a kit including one or more of the noted ingredients (and optionally the target or target sequence or sample).

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art to which this invention belongs. See, for example Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). It is to be understood that both the general description and the detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

In some embodiments, any one or more of the optional elements of any one or more of the figures herein can be combined with any one or more of the other optional elements of any one or more of the figures herein. In some embodiments, any one or more of the compositions or steps provided in any of the figures provided herein can be combined with any of the other compositions or steps provided herein. As used herein, a generic reference to a set of figures (for example, FIG. 36) denotes all of the different figures contained within that number (for example, FIGS. 36A-36R), each combined together, one or more of them, or each in the alternative, unless otherwise denoted.

In some embodiments, any one or more of the compositions or steps provided in any of the figures provided herein can be combined with any of the other compositions or steps provided herein. As used herein, a generic reference to a set of figures (for example, FIG. 2) denotes all of the different figures contained within that number (for example, FIGS. 2A-2D), each combined together, one or more of them, or each in the alternative, unless otherwise denoted.

### REFERENCES

ADDIN ZOTERO_BIBL {“uncited”: [ ], “omitted”: [ ], “custom”: [ ]} CSL_BIBLIOGRAPHY
(1) Dirks, R. M.; Pierce, N. A. Triggered Amplification by Hybridization Chain Reaction. Proc. Natl. Acad. Sci. U.S.A 2004, 101 (43), 15275-15278.
(2) Li, G.; Moellering, R. E. A Concise, Modular Antibody—Oligonucleotide Conjugation Strategy Based on Disuccinimidyl Ester Activation Chemistry. ChemBioChem 2019, 20 (12), 1599-1605.
(3) Cal, P. M. S. D.; Bernardes, G. J. L.; Gois, P. M. P. Cysteine-Selective Reactions for Antibody Conjugation. Angew. Chem. Int. Ed. 2014, 53 (40), 10585-10587.
(4) Chen, F.; Gao, J. Fast Cysteine Bioconjugation Chemistry. Chem.—Eur. J. 2022, 28 (66), e202201843.
(5) Dawson, P. E.; Muir, T. W.; Clark-Lewis, I.; Kent, S. B. H. Synthesis of Proteins by Native Chemical Ligation. Science 1994, 266 (5186), 776-779.
(6) Haldón, E.; Nicasio, M. C.; Pérez, P. J. Copper-Catalysed Azide-Alkyne Cycloadditions (CuAAC): An Update. Org. Biomol. Chem. 2015, 13 (37), 9528-9550.
(7) Agard, N. J.; Prescher, J. A.; Bertozzi, C. R. A Strain-Promoted [3+2] Azide-Alkyne Cycloaddition for Covalent Modification of Biomolecules in Living Systems. J. Am. Chem. Soc. 2004, 126 (46), 15046-15047.
(8) Ulrich, S.; Boturyn, D.; Marra, A.; Renaudet, O.; Dumy, P. Oxime Ligation: A Chemoselective Click-Type Reaction for Accessing Multifunctional Biomolecular Constructs. Chem.—Eur. J. 2014, 20 (1), 34-41.
(9) Johann, K.; Svatunek, D.; Seidl, C.; Rizzelli, S.; Bauer, T. A.; Braun, L.; Koynov, K.; Mikula, H.; Barz, M. Tetrazine- and Trans-Cyclooctene-Functionalised Polypept (o) Ides for Fast Bioorthogonal Tetrazine Ligation. Polym. Chem. 2020, 11 (27), 4396-4407.
(10) Zaman, U.; Richter, Florian M.; Hofele, R.; Kramer, K.; Sachsenberg, T.; Kohlbacher, O.; Lenz, C.; Urlaub, H. Dithiothreitol (DTT) Acts as a Specific, UV-Inducible Cross-Linker in Elucidation of Protein-RNA Interactions*. Mol. Cell. Proteomics 2015, 14 (12), 3196-3210.
(11) Hui, J. Z.; Tamsen, S.; Song, Y.; Tsourkas, A. LASIC: Light Activated Site-Specific Conjugation of Native IgGs. Bioconjug. Chem. 2015, 26 (8), 1456-1460.
(12) Watson, E. E.; Winssinger, N. Synthesis of Protein-Oligonucleotide Conjugates. Biomolecules 2022, 12 (10), 1523.
(13) Lovendahl, K. N.; Hayward, A. N.; Gordon, W. R. Sequence-Directed Covalent Protein—DNA Linkages in a Single Step Using HUH-Tags. J. Am. Chem. Soc. 2017, 139 (20), 7030-7035.
(14) Los, G. V.; Encell, L. P.; McDougall, M. G.; Hartzell, D. D.; Karassina, N.; Zimprich, C.; Wood, M. G.; Learish, R.; Ohana, R. F.; Urh, M.; Simpson, D.; Mendez, J.; Zimmerman, K.; Otto, P.; Vidugiris, G.; Zhu, J.; Darzins, A.; Klaubert, D. H.; Bulleit, R. F.; Wood, K. V. HaloTag: A Novel Protein Labeling Technology for Cell Imaging and Protein Analysis. ACS Chem. Biol. 2008, 3 (6), 373-382.
(15) Keppler, A.; Gendreizig, S.; Gronemeyer, T.; Pick, H.; Vogel, H.; Johnsson, K. A General Method for the Covalent Labeling of Fusion Proteins with Small Molecules in Vivo. Nat. Biotechnol. 2003, 21 (1), 86-89.
(16) Qian, X.; Jin, L.; Lloyd, R. V. In Situ Hybridization: Basic Approaches and Recent Development. J. Histotechnol. 2004, 27 (1), 53-67.
(17) Silverman, A.; Kool, E. Oligonucleotide Probes for RNA-Targeted Fluorescence in Situ Hybridization. Adv. Clin. Chem. 2007, 43, 79-115.
(18) Ramos-Vara, J. A.; Miller, M. A. When Tissue Antigens and Antibodies Get along: Revisiting the Technical Aspects of Immunohistochemistry—the Red, Brown, and Blue Technique. Vet. Pathol. 2014, 51 (1), 42-87.
(19) Choi, H. M. T.; Chang, J. Y.; Trinh, L. A.; Padilla, J. E.; Fraser, S. E.; Pierce, N. A. Programmable in Situ Amplification for Multiplexed Imaging of mRNA Expression. Nat. Biotechnol. 2010, 28 (11), 1208-1212.
(20) Choi, H. M. T.; Beck, V. A.; Pierce, N. A. Next-Generation in Situ Hybridization Chain Reaction: Higher Gain, Lower Cost, Greater Durability. ACS Nano 2014, 8 (5), 4284-4294.
(21) Choi, H. M. T.; Schwarzkopf, M.; Fornace, M. E.; Acharya, A.; Artavanis, G.; Stegmaier, J.; Cunha, A.; Pierce, N. A. Third-Generation in Situ Hybridization Chain Reaction: Multiplexed, Quantitative, Sensitive, Versatile, Robust. Development 2018, 145, dev165753.
(22) Schwarzkopf, M.; Liu, M. C.; Schulte, S. J.; Ives, R.; Husain, N.; Choi, H. M. T.; Pierce, N. A. Hybridization Chain Reaction Enables a Unified Approach to Multiplexed, Quantitative, High-Resolution Immunohistochemistry and in Situ Hybridization. Development 2021, 148 (22), dev199847.
(23) Choi, H. M. T.; Calvert, C. R.; Husain, N.; Huss, D.; Barsi, J. C.; Deverman, B. E.; Hunter, R. C.; Kato, M.; Lee, S. M.; Abelin, A. C. T.; Rosenthal, A. Z.; Akbari, O. S.; Li, Y.; Hay, B. A.; Sternberg, P. W.; Patterson, P. H.; Davidson, E. H.; Mazmanian, S. K.; Prober, D. A.; van de Rijn, M.; Leadbetter, J. R.; Newman, D. K.; Readhead, C.; Bronner, M. E.; Wold, B.; Lansford, R.; Sauka-Spengler, T.; Fraser, S. E.; Pierce, N. A. Mapping a Multiplexed Zoo of mRNA Expression. Development 2016, 143, 3632-3637.
(24) Collins, M. L.; Irvine, B.; Tyner, D.; Fine, E.; Zayati, C.; Chang, C. A.; Horn, T.; Ahle, D.; Detmer, J.; Shen, L.-P.; Kolberg, J.; Bushnell, S.; Urdea, M. S.; Ho, D. D. A Branched DNA Signal Amplification Assay for Quantification of Nucleic Acid Targets below 100 Molecules/Ml. Nucleic Acids Res. 1997, 25 (15), 2979-2984.
(25) Bushnell, S.; Budde, J.; Catino, T.; Cole, J.; Derti, A.; Kelso, R.; Collins, M. L.; Molino, G.; Sheridan, P.; Monahan, J.; Urdea, M. ProbeDesigner: For the Design of Probesets for Branched DNA (bDNA) Signal Amplification Assays. Bioinformatics 1999, 15 (5), 348-355.
(26) Player, A. N.; Shen, L.-P.; Kenny, D.; Antao, V. P.; Kolberg, J. A. Single-Copy Gene Detection Using Branched DNA (bDNA) in Situ Hybridization. J. Histochem. Cytochem. 2001, 49 (5), 603-611.
(27) Qian, X.; Lloyd, R. V. Recent Developments in Signal Amplification Methods for in Situ Hybridization. Diagn. Mol. Pathol. 2003, 12 (1), 1-13.
(28) Wang, F.; Flanagan, J.; Su, N.; Wang, L.-C.; Bui, S.; Nielson, A.; Wu, X. Y.; Vo, H.-T.; Ma, X.-J.; Luo, Y. L. RNAscope: A Novel in Situ RNA Analysis Platform for Formalin-Fixed, Paraffin-Embedded Tissues. J. Mol. Diagn. 2012, 14 (1), 22-29.
(29) Kishi, J. Y.; Lapan, S. W.; Beliveau, B. J.; West, E. R.; Zhu, A.; Sasaki, H. M.; Saka, S. K.; Wang, Y.; Cepko, C. L.; Yin, P. SABER Amplifies FISH: Enhanced Multiplexed Imaging of RNA and DNA in Cells and Tissues. Nat Methods 2019, 16 (6), 533-544.
(30) Saka, S. K.; Wang, Y.; Kishi, J. Y.; Zhu, A.; Zeng, Y.; Xie, W.; Kirli, K.; Yapp, C.; Cicconet, M.; Beliveau, B. J.; Lapan, S. W.; Yin, S.; Lin, M.; Boyden, E. S.; Kaeser, P. S.; Pihan, G.; Church, G. M.; Yin, P. Immuno-SABER Enables Highly Multiplexed and Amplified Protein Imaging in Tissues. Nat. Biotechnol. 2019, 37 (9), 1080-1090.
(31) Zhou, Y.; Calciano, M.; Hamann, S.; Leamon, J. H.; Strugnell, T.; Christian, M. W.; Lizardi, P. M. In Situ Detection of Messenger RNA Using Digoxigenin-Labeled Oligonucleotides and Rolling Circle Amplification. Exp. Mol. Pathol. 2001, 70, 281-288.
(32) Schweitzer, B.; Kingsmore, S. Combining Nucleic Acid Amplification and Detection. Curr. Opin. Biotechnol. 2001, 12, 21-27.
(33) Larsson, C.; Koch, J.; Nygren, A.; Janssen, G.; Raap, A. K.; Landegren, U.; Nilsson, M. In Situ Genotyping Individual DNA Molecules by Target-Primed Rolling-Circle Amplification of Padlock Probes. Nat. Methods 2004, 1 (3), 227-232.
(34) Zhou, H.; Bouwman, K.; Schotanus, M.; Verweij, C.; Marrero, J. A.; Dillon, D.; Costa, J.; Lizardi, P.; Haab, B. B. Two-Color, Rolling-Circle Amplification on Antibody Microarrays for Sensitive, Multiplexed Serum-Protein Measurements. Genome Biol. 2004, 5 (4), R28.
(35) Larsson, C.; Grundberg, I.; Soderberg, O.; Nilsson, M. In Situ Detection and Genotyping of Individual mRNA Molecules. Nat. Methods, 2010, 7, 395-397.
(36) Tautz, D.; Pfeifle, C. A Non-Radioactive in Situ Hybridization Method for the Localization of Specific RNAs in Drosophila Embryos Reveals Translational Control of the Segmentation Gene Hunchback. Chromosoma 1989, 98, 81-85.
(37) Harland, R. M. In Situ Hybridization: An Improved Whole-Mount Method for Xenopus Embryos. Methods Cell Biol. 1991, 36, 685-695.
(38) Lehmann, R.; Tautz, D. In Situ Hybridization to RNA. In Drosophila melanogaster: practical uses in cell and molecular biology; Goldstein, L. S. B., Fyrberg, E. A., Eds.; Methods in Cell Biology; Elsevier Academic Press: San Diego, CA, 1994; Vol. 44, pp 575-598.
(39) Kerstens, H. M. J.; Poddighe, P. J.; Hanselaar, A. G. J. M. A Novel In-Situ Hybridization Signal Amplification Method Based on the Deposition of Biotinylated Tyramine. J. Histochem. Cytochem. 1995, 43 (4), 347-352.
(40) Nieto, M. A.; Patel, K.; Wilkinson, D. G. In Situ Hybridization Analysis of Chick Embryos in Whole Mount and Tissue Sections. In Methods in Avian Embryology; Bronner-Fraser, M., Ed.; Methods in Cell Biology; Elsevier Academic Press: San Diego, CA, 1996; Vol. 51, pp 219-235.
(41) Pernthaler, A.; Pernthaler, J.; Amann, R. Fluorescence in Situ Hybridization and Catalyzed Reporter Deposition for the Identification of Marine Bacteria. Appl. Environ. Microbiol. 2002, 68 (6), 3094-3101.
(42) Kosman, D.; Mizutani, C. M.; Lemons, D.; Cox, W. G.; McGinnis, W.; Bier, E. Multiplex Detection of RNA Expression in Drosophila Embryos. Science 2004, 305, 846.
(43) Thisse, B.; Heyer, V.; Lux, A.; Alunni, V.; Degrave, A.; Seiliez, I.; Kirchner, J.; Parkhill, J. P.; Thisse, C. Spatial and Temporal Expression of the Zebrafish Genome by Large-Scale in Situ Hybridization Screening. In The Zebrafish: 2nd Edition Genetics Genomics and Informatics; Detrich III, H. W. D., Zon, L. I., Westerfield, M., Eds.; Methods in Cell Biology; Elsevier Academic Press: San Diego, CA, 2004; Vol. 77, pp 505-519.
(44) Denkers, N.; Garcia-Villalba, P.; Rodesch, C. K.; Nielson, K. R.; Mauch, T. J. FISHing for Chick Genes: Triple-label Whole-mount Fluorescence in Situ Hybridization Detects Simultaneous and Overlapping Gene Expression in Avian Embryos. Dev. Dyn. 2004, 229 (3), 651-657.
(45) Clay, H.; Ramakrishnan, L. Multiplex Fluorescent in Situ Hybridization in Zebrafish Embryos Using Tyramide Signal Amplification. Zebrafish 2005, 2 (2), 105-111.
(46) Barroso-Chinea, P.; Aymerich, M. S.; Castle, M. M.; Perez-Manso, M.; Tunon, T.; Erro, E.; Lanciego, J. L. Detection of Two Different mRNAs in a Single Section by Dual in Situ Hybridization: A Comparison between Colorimetric and Fluorescent Detection. J. Neurosci. Methods 2007, 162 (1-2), 119-128.
(47) Acloque, H.; Wilkinson, D. G.; Nieto, M. A. In Situ Hybridization Analysis of Chick Embryos in Whole-Mount and Tissue Sections. In Avian Embryology, 2nd Edition; Bronner-Fraser, M., Ed.; Methods in Cell Biology; Elsevier Academic Press: San Diego, CA, 2008; Vol. 87, pp 169-185.
(48) Piette, D.; Hendrickx, M.; Willems, E.; Kemp, C. R.; Leyns, L. An Optimized Procedure for Whole-Mount in Situ Hybridization on Mouse Embryos and Embryoid Bodies. Nat. Protoc. 2008, 3 (7), 1194-1201.
(49) Thisse, C.; Thisse, B. High-Resolution in Situ Hybridization to Whole-Mount Zebrafish Embryos. Nat. Protoc. 2008, 3 (1), 59-69.
(50) Weiszmann, R.; Hammonds, A. S.; Celniker, S. E. Determination of Gene Expression Patterns Using High-Throughput RNA in Situ Hybridization to Whole-Mount Drosophila Embryos. Nat. Protoc. 2009, 4 (5), 605-618.
(51) Takakura, N.; Yoshida, H.; Ogura, Y.; Kataoka, H.; Nishikawa, S.; Nishikawa, S. PDGFR Alpha Expression during Mouse Embryogenesis: Immunolocalization Analyzed by Whole-Mount Immunohistostaining Using the Monoclonal Anti-Mouse PDGFR Alpha Antibody APA5. J. Histochem. Cytochem. 1997, 45 (6), 883-893.
(52) Sillitoe, R. V.; Hawkes, R. Whole-Mount Immunohistochemistry: A High-Throughput Screen for Patterning Defects in the Mouse Cerebellum. J. Histochem. Cytochem. 2002, 50 (2), 235-244.
(53) Ahnfelt-Ronne, J.; Jorgensen, M. C.; Hald, J.; Madsen, O. D.; Serup, P.; Hecksher-Sorensen, J. An Improved Method for Three-Dimensional Reconstruction of Protein Expression Patterns in Intact Mouse and Chicken Embryos and Organs. J. Histochem. Cytochem. 2007, 55 (9), 925-930.
(54) Psychoyos, D.; Finnell, R. Method for Whole Mount Antibody Staining in Chick. J. Vis. Exp. 2009, No. 24, e956.
(55) Fujisawa, S.; Yarilin, D.; Fan, N.; Turkekul, M.; Xu, K.; Barlas, A.; Manova-Todorova, K. Understanding the Three-Dimensional World from Two-Dimensional Immunofluorescent Adjacent Sections. J. Pathol. Inform. 2015, 6, 27.
(56) Staudt, N.; Muller-Sienerth, N.; Fane-Dremucheva, A.; Yusaf, S. P.; Millrine, D.;
Wright, G. J. A Panel of Recombinant Monoclonal Antibodies against Zebrafish Neural
Receptors and Secreted Proteins Suitable for Wholemount Immunostaining. Biochem. Biophys. Res. Commun. 2015, 456 (1), 527-533.
(57) Wiedorn, K. H.; Kuhl, H.; Galle, J.; Caselitz, J.; Vollmer, E. Comparison of In-Situ Hybridization, Direct and Indirect in-Situ PCR as Well as Tyramide Signal Amplification for the Detection of HPV. Histochem. Cell Biol. 1999, 111, 89-95.
(58) Soderberg, O.; Gullberg, M.; Jarvius, M.; Ridderstrale, K.; Leuchowius, K. J.; Jarvius, J.; Wester, K.; Hydbring, P.; Bahram, F.; Larsson, L. G.; Landegren, U. Direct Observation of Individual Endogenous Protein Complexes in Situ by Proximity Ligation. Nat Methods, 2006, 3, 995-1000.
(59) Weibrecht, I.; Gavrilovic, M.; Lindbom, L.; Landegren, U.; Wahlby, C.; Söderberg, O. Visualising Individual Sequence-Specific Protein-DNA Interactions in Situ. New Biotechnol. 2012, 29 (5), 589-598.
(60) Jung, J.; Lifland, A. W.; Zurla, C.; Alonas, E. J.; Santangelo, P. J. Quantifying RNA-Protein Interactions in Situ Using Modified-MTRIPs and Proximity Ligation. Nucleic Acids Res 2013, 41 (1), e12.
(61) Gomez, D.; Shankman, L. S.; Nguyen, A. T.; Owens, G. K. Detection of Histone Modifications at Specific Gene Loci in Single Cells in Histological Sections. Nat Methods 2013, 10 (2), 171-177.
(62) Leuchowius, K. J.; Clausson, C. M.; Grannas, K.; Erbilgin, Y.; Botling, J.; Zieba, A.; Landegren, U.; Söderberg, O. Parallel Visualization of Multiple Protein Complexes in Individual Cells in Tumor Tissue. Mol. Cell. Proteomics 2013, 12 (6), 1563-1571.
(63) Bellucci, A.; Fiorentini, C.; Zaltieri, M.; Missale, C.; Spano, P. The in Situ Proximity Ligation Assay to Probe Protein-Protein Interactions in Intact Tissues. Methods Mol Biol 2014, 1174, 397-405.
(64) Koos, B.; Andersson, L.; Clausson, C. M.; Grannas, K.; Klaesson, A.; Cane, G.; Soderberg, O. Analysis of Protein Interactions in Situ by Proximity Ligation Assays. Curr Top Microbiol Immunol, 2014, 377, 111-126.
(65) Roussis, I. M.; Guille, M.; Myers, F. A.; Scarlett, G. P. RNA Whole-Mount in Situ Hybridisation Proximity Ligation Assay (rISH-PLA), an Assay for Detecting RNA-Protein Complexes in Intact Cells. PLOS One 2016, 11 (1), e0147967.
(66) Petruk, S.; Fenstermaker, T. K.; Black, K. L.; Brock, H. W.; Mazo, A. Detection of RNA-DNA Association by a Proximity Ligation-Based Method. Sci Rep 2016, 6, 27313.
(67) Black, K. L.; Petruk, S.; Fenstermaker, T. K.; Hodgson, J. W.; Caplan, J. L.; Brock, H. W.; Mazo, A. Chromatin Proteins and RNA Are Associated with DNA during All Phases of Mitosis. Cell Discov 2016, 2, 16038.
(68) Zurla, C.; Jung, J.; Blanchard, E. L.; Santangelo, P. J. A Novel Method to Quantify RNA-Protein Interactions in Situ Using FMTRIP and Proximity Ligation. Methods Mol Biol 2017, 1468, 155-170.
(69) Klaesson, A.; Grannas, K.; Ebai, T.; Heldin, J.; Koos, B.; Leino, M.; Raykova, D.; Oelrich, J.; Arngården, L.; Söderberg, O.; Landegren, U. Improved Efficiency of in Situ Protein Analysis by Proximity Ligation Using UnFold Probes. Sci. Rep. 2018, 8 (1), 5400.
(70) Schulte, S. J.; Shin, B.; Rothenberg, E. V.; Pierce, N. A. Multiplex, Quantitative, High-Resolution Imaging of Protein: Protein Complexes via Hybridization Chain Reaction. ACS Chem. Biol. 2024, 19 (2), 280-288.
(71) Schulte, S. J.; Fornace, M. E.; Hall, J. K.; Shin, G. J.; Pierce, N. A. HCR Spectral Imaging: 10-Plex, Quantitative, High-Resolution RNA and Protein Imaging in Highly Autofluorescent Samples. Development 2024, 151, dev202307.

1-17. (canceled)

18. A method for amplified detection of a target in a sample, the method comprising:
a) providing a sample containing a target;
b) providing a probe set comprising either:
i) an HCR initiator-labeled probe, or
ii) a probe unit comprising two or more HCR fractional-initiator probes;
c) providing a reporter-labeled first HCR amplifier;
d) generating a signal, directly or indirectly, from one or more reporter-decorated HCR amplification polymers;
e) detecting the signal;
wherein an HCR initiator-labeled probe comprises:
a barcode probe comprising: i) a primary probe comprising a target-binding domain and ii) a secondary probe comprising a primary-probe-binding domain and a conjugation site; wherein the secondary probe is bound to the primary probe and the conjugation site is covalently coupled to a barcode oligonucleotide comprising an HCR initiator,
wherein an HCR fractional-initiator probe comprises:
a barcode probe comprising: i) a primary probe comprising a target-binding domain and ii) a secondary probe comprising a primary-probe-binding domain and a conjugation site; wherein the secondary probe is bound to the primary probe and the conjugation site is covalently coupled to a barcode oligonucleotide comprising an HCR fractional initiator;
wherein an HCR amplifier comprises two or more HCR hairpins;
wherein an HCR hairpin comprises:
an input domain comprising:
a single-stranded toehold and
a stem section,
wherein an HCR hairpin further comprises an output domain comprising:
a single-stranded loop and
a complement to the stem section, and
wherein at least one HCR hairpin further comprises one or more reporters; and
wherein when the HCR initiator-labeled probe or probe unit is bound to the target the HCR initiator or colocalized full initiator comprising two or more HCR fractional-initiators initiates HCR signal amplification whereupon the HCR hairpins self-assemble into a tethered reporter-decorated HCR amplification polymer thereby generating the signal.

19. The method of claim 18, wherein a wash step is performed between steps b) and c).

20. The method of claim 18, wherein a wash step is performed between steps c) and d).

21. The method of claim 18, wherein the signal is removed from the sample following step e).

22. The method of claim 18, wherein the reporters on the reporter-decorated amplification polymers directly or indirectly mediate CARD signal amplification.

23. The method of claim 18, wherein the target comprises a protein, a peptide, an amino acid, a non-natural amino acid analog, a nucleic acid, a non-natural nucleic acid analog, a tag, a hapten, a fluorophore, a reporter, a chemical, a biological molecule, a pathogen, a small molecule, a macromolecule, a complex of molecules, or a set of molecules in proximity.

24. The method of claim 18, wherein the probe unit comprising two or more HCR fractional-initiator probes further comprises one or more proximity probes.

25. The method of claim 24, wherein each HCR fractional-initiator probe within a probe unit further comprises a proximity domain.

26. The method of claim 25, wherein the one or more proximity probes bind to the proximity domains within a probe unit to colocalize a full HCR initiator capable of triggering HCR signal amplification.

27. The method of claim 18, further comprising repeating any of the steps of the method to detect another target in the sample.

28-46. (canceled)

47. A method for amplified detection of a target in a sample, the method comprising:
a) providing a sample containing a target;
b) providing a probe set comprising either:
i) an HCR initiator-labeled fusion probe, or
ii) a probe unit comprising two or more HCR fractional-initiator fusion probes;
c) providing a reporter-labeled first HCR amplifier;
d) generating a signal, directly or indirectly, from one or more reporter-decorated HCR amplification polymers;
e) detecting the signal;
wherein an HCR initiator-labeled fusion probe comprises:
a barcode fusion probe comprising a primary probe comprising a target-binding domain wherein either the primary probe or a secondary probe bound to the primary probe comprises a fusion to an enzymatic conjugation domain covalently coupled to a barcode oligonucleotide comprising an HCR initiator,
wherein an HCR fractional-initiator fusion probe comprises:
a barcode fusion probe comprising a primary probe comprising a target-binding domain wherein either the primary probe or a secondary probe bound to the primary probe comprises a fusion to an enzymatic conjugation domain covalently coupled to a barcode oligonucleotide comprising an HCR fractional initiator;
wherein an HCR amplifier comprises two or more HCR hairpins;
wherein an HCR hairpin comprises:
an input domain comprising:
a single-stranded toehold and
a stem section,
wherein an HCR hairpin further comprises an output domain comprising:
a single-stranded loop and
a complement to the stem section, and
wherein at least one HCR hairpin further comprises one or more reporters;
wherein when the HCR initiator-labeled probe or probe unit is bound to the target the HCR initiator or colocalized full initiator comprising two or more HCR fractional-initiators initiates HCR signal amplification whereupon the HCR hairpins self-assemble into a tethered reporter-decorated HCR amplification polymer thereby generating the signal.

48. The method of claim 47, wherein a wash step is performed between steps b) and c).

49. The method of claim 47, wherein a wash step is performed between steps c) and d).

50. The method of claim 47, wherein the signal is removed from the sample following step e).

51. The method of claim 47, wherein the target comprises a protein, a peptide, an amino acid, a non-natural amino acid analog, a nucleic acid, a non-natural nucleic acid analog, a tag, a hapten, a fluorophore, a reporter, a chemical, a biological molecule, a pathogen, a small molecule, a macromolecule, a complex of molecules, or a set of molecules in proximity.

52. The method of claim 47, wherein the probe unit comprising two or more HCR fractional-initiator probes further comprises one or more proximity probes.

53. The method of claim 52, wherein each HCR fractional-initiator probe within a probe unit further comprises a proximity domain.

54. The method of claim 53, wherein the one or more proximity probes bind to the proximity domains within a probe unit to colocalize a full HCR initiator capable of triggering HCR signal amplification.

55. The method of claim 47, further comprising repeating any of the steps of the method to detect another target in the sample.

56. The method of claim 47, wherein the reporters on the reporter-decorated amplification polymers directly or indirectly mediate CARD signal amplification.


SEQUENCE LISTING (2 sequences)

SEQ ID NO: 1 - protein, 120 aa, synthetic construct
MAKSGNYSYKRWVFTINNPTFEDYVHVLEFCTLDNCKFAIVGEEKGANGTPHLQGFLNLRSNARAAALEESLGGRAWLSRARGSDEDNEEYCAKESTYLRVGEPVSKGRSSDLAEATSAV

SEQ ID NO: 2 - DNA, 26 bp, synthetic construct
  source 1..26: mol_type = other DNA
aaagtattaccagaagagtattgaac